(19) United States
(12) Patent Application Publication
(10) Pub. No.: US 2013/0310384 A1
(43) Pub. Date: Nov. 21, 2013

(54) Sulfonamide-Containing Compounds

(71) Applicants: Corinne E. Augelli-Szafran, Newton, MA (US); Dai Lu, Boston, MA (US); Hanxun Wei, West Roxbury, MA (US); Jing Zhang, Lexington, MA (US); Michael S. Wolfe, Newton, MA (US); Dennis J. Selkoe, Jamaica Plain, MA (US)

(72) Inventors: Corinne E. Augelli-Szafran, Newton, MA (US); Dai Lu, Boston, MA (US); Hanxun Wei, West Roxbury, MA (US); Jing Zhang, Lexington, MA (US); Michael S. Wolfe, Newton, MA (US); Dennis J. Selkoe, Jamaica Plain, MA (US)

(73) Assignee: THE BRIGHAM AND WOMEN'S HOSPITAL, INC., Boston, MA (US)

(21) Appl. No.: 13/877,419

(22) PCT Filed: Oct. 4, 2011
(86) PCT No.: PCT/US11/54812

§ 371 Date: Aug. 1, 2013

Related U.S. Application Data
(60) Provisional application No. 61/389,537, filed on Oct. 4, 2010.

(51) Int. Cl. C07C 311/16; C07C 311/18; C07D 295/32; C07D 263/14; C07D 213/42; C07C 311/19; C07D 271/06

(52) U.S. Cl. CPC C07C 311/16; C07C 311/19; C07C 311/18; C07D 271/06; C07D 263/14; C07D 213/42; C07D 295/32
USPC 514/238.8; 564/92; 514/604; 548/131; 514/364; 548/238; 514/374; 546/338; 514/357; 544/159

(57) ABSTRACT

This invention relates generally to the discovery of sulfonamide-containing compounds that are inhibitors of γ-secretase.

### CROSS REFERENCE TO RELATED APPLICATIONS

This application claims the benefit of U.S. Provisional Application No. 61/389,537, filed on Oct. 4, 2010, which is incorporated herein by reference in its entirety.

### STATEMENT REGARDING FEDERALLY SPONSORED RESEARCH

This work was supported by grants NS41355 and AG15379 from the National Institutes of Health. The Government has certain rights in the invention.

### TECHNICAL FIELD

This invention relates generally to the discovery of sulfonamide-containing compounds that are inhibitors of γ-secretase.

### BACKGROUND

Accumulating biochemical, histological, and genetic evidence supports the hypothesis that the 4 kDa β-amyloid protein (Aβ) is an essential component in the pathogenesis of Alzheimer's disease (“AD”). Selkoe D J, Science 275:630-631 (1997). Hardy J, Proc Natl Acad Sci USA 94:2095-2097 (1997). Despite the intense interest in the role of Aβ in the etiology of AD, the molecular mechanism of Aβ biosynthesis is still not fully understood. The 39-43-residue Aβ is formed via the sequential cleavage of the integral membrane amyloid precursor protein (APP) by (3- and γ-secretases. Selkoe D J, Annu Rev Cell Biol 10:373-403 (1994). β-Secretase cleavage of APP occurs near the membrane, producing the soluble APPg-β and a 12 kDa C-terminal membrane-associated fragment (CTF). The latter is processed by γ-secretase that cleaves within the transmembrane domain of the substrate to generate Aβ. An alternative proteolytic event carried out by α-secretase occurs within the Aβ portion of APP, releasing APPg-α. Subsequent processing of the resulting membrane-bound 10 kDa CTF by γ-secretase leads to the formation of a 3 kDa N-terminally truncated version of Aβ called p3.

Heterogeneous proteolysis of the 12 kDa CTF by γ-secretase generates primarily two C-terminal variants of Aβ, 40- and 42-amino acid versions (Aβ40 and Aβ42), and parallel processing of the 10 kDa CTF generates the corresponding C-terminal variants of p3. Although Aβ42 represents only about 10% of secreted Aβ, this longer and more hydrophobic variant is disproportionally present in the amyloid plaques observed post mortem in AD patients (Roher A E et al., Proc Natl Acad Sci USA 90:10836-40 (1993); Iwatsubo T et al., Neuron 13:45-53 (1994)) which is consistent with in vitro studies illustrating the kinetic insolubility of Aβ42 vis-a-vis Aβ40. Jarrett J T et al., Biochemistry 32:4693-4697 (1993). Importantly, all genetic mutations associated with early-onset (<60 years) familial Alzheimer's disease (FAD) result in increased Aβ42 production. Selkoe D J, Science 275:630-631 (1997); Hardy J, Proc Natl Acad Sci USA 94:2095-2097 (1997).

γ-secretase is therefore believed to be an attractive target for inhibitor design for the purpose of inhibiting production of Aβ and treating disorders characterized by the production and deposition of β-amyloid.

### SUMMARY

This invention relates generally to the discovery of sulfonamide-containing compounds that are inhibitors of γ-secretase.

As used herein, it should be appreciated that the term “inhibitor” refers to a compound that modulates (e.g., reduces) the activity of its target (e.g., protease) regardless of the mode of action of the inhibitor. Accordingly, in some embodiments, an inhibitor may react at the active site (e.g., catalytic site) of a protease thereby reducing its activity (e.g., inactivating the protease). In some embodiments, an inhibitor may be a transition state inhibitor. In some embodiments, an inhibitor may be a modulator (e.g., an allosteric modulator) that inhibits protease activity by binding to a modulatory site that indirectly alters the conformation of the active site, substrate binding site, or other site (or combination thereof) thereby modulating the activity of the protease (e.g., reducing the activity of the protease, changing the specificity of the protease, etc., or any combination thereof). In some embodiments, an inhibitor may modulate protease activity either by binding to the protease or to a substrate (or a combination thereof) thereby reducing the activity of the protease for the substrate. In some embodiments, an inhibitor may bind to the protease at a position that interferes with one or more substrate binding and/or product release steps. It should be appreciated that aspects of the invention are not limited by the precise mode of action of the inhibitor and that any direct or indirect effect on the activity of a protease may result from contacting γ-secretase with an inhibitor of the invention. In some embodiments, without wishing to be limited by theory, an inhibitor of the invention may bind to a proposed modulatory site on γ-secretase (see, e.g., Lazarov et. al., P.N.A.S., vol. 103, p. 6889). It also should be appreciated that an inhibitor of the invention may partially or completely inhibit the secretase activity (e.g., by about 10%, about 20%, about 30%, about 40%, about 50%, about 60%, about 70%, about 80%, about 90%, about 95%, or by less or more than any of these values, for example, by 100%, or by any intermediate percentage). In some embodiments, inhibition may be specific (e.g., substrate specific) in that the inhibitory effect is stronger for a first substrate than a second substrate. In some embodiments, specific inhibitors of the invention reduce degradation of the amyloid precursor protein to a greater extent than that of the Notch protein (e.g., the ratio of % inhibition of amyloid precursor protein degradation to % inhibition of Notch protein degradation is greater than 1). In some embodiments, amyloid precursor protein degradation by γ-secretase may be inhibited by a compound of the invention, whereas Notch degradation by γ-secretase may be unaffected or only slightly inhibited. Certain aspartyl proteases, including γ-secretase, generate β-amyloid from amyloid precursor protein (APP) which may result in neurodegenerative disorders. The γ-secretase inhibitor compounds are useful for treating a subject having or at risk of developing a neurodegenerative disorder associated with γ-secretase activity, e.g., Alzheimer's disease. In some aspects, specific inhibitors of the invention may be used to treat or prevent Alzheimer's disease without causing side effects associated with inhibition of Notch degradation.

The invention also features compositions (e.g., pharmaceutical compositions) and articles of manufacture that include one of more of the compounds described herein as well as methods of making, identifying, and using such compounds.

Other features and advantages are described in, or will be apparent from, the present specification and accompanying FIGURE.

[I] Accordingly, in one aspect, compounds having formula (I) are featured:

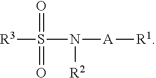

[A] In some embodiments:

### R¹ is:

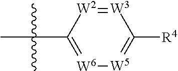

wherein:
W², W³, W⁵, and W⁶ are defined according to (A) or (B) below:
(A) each of W², W³, W⁵, and W⁶ is independently selected from CH or C(halo) (in some embodiments, the definition of W², W³, W⁵, and W⁶ can further include COR (where R═H, C₁-C₆ alkyl); or
(B) one or two of W², W³, W⁵, and W⁶ are N; and the others are independently selected from CH or C(halo);
R⁴ is selected from:
(i) halo; —CO₂H; —C(O)OR⁴¹; —NHC(O)OR⁴¹; —N(CH₃)C(O)OR⁴¹;
—C(O)N(R⁴²)(R⁴³); —C(O)R⁴⁴; —CN; —NO₂; —SO₃H; —P(O)(OH)₂; —OH, —SO₂(R⁴⁵); —NHC(O)R⁴¹, —NHSO₂R⁴¹, —SO₂N(R⁴²)(R⁴³); —C(O)NHCH(CH₂OH)₂, —C(O)NH(CH₂)₃COOH,
(ii) C₁-C₆ alkoxy, —OCH(CH₂OH)₂, C₁-C₆ thioalkoxy, C₁-C₆ haloalkoxy, C₁-C₆ halothioalkoxy, C₁-C₆ alkyl, C₁-C₆ haloalkyl, each of which is optionally substituted with from 1-3 (e.g., 1-2 or 1) substituents independently selected from —OH and —CN;
(iii) heterocyclyl, each containing from 3-8 ring atoms, wherein from 1-2 of the ring atoms is independently selected from N, NH, N(C₁-C₆ alkyl), O, and S; and wherein said heterocyclic ring is optionally substituted with from 1-3 independently selected R^{a};
(iv) heterocycloalkenyl or heteroaryl, each containing 5 ring atoms, wherein from 1-4 of the ring atoms is independently selected from N, NH, N(C₁-C₆ alkyl), O, and S; and wherein said heteroaryl ring is optionally substituted with from 1-3 independently selected le; and
(v) hydrogen;
R⁴¹ is C₁-C₈ alkyl, C₁-C₈ haloalkyl, or benzyl optionally substituted with from 1-3 R^{b};
each of R⁴² and R⁴³ is independently selected from hydrogen; C₁-C₈ alkyl or C₁-C₈ haloalkyl, each of which is optionally substituted with from 1-3 substituents independently selected from —OH; OCH₃, CN, COOH, and —NHC(O)(C₁-C₃ alkyl);
R⁴⁴ is hydrogen, C₁-C₈ alkyl, or C₁-C₈ haloalkyl;
R⁴⁵ is C₁-C₈ alkyl or C₁-C₈ haloalkyl;
   A is C(R^{A})₂, wherein each occurrence of R^{A} is independently selected from hydrogen and —CH₃;

### R² is:

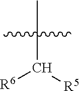

wherein
R⁵ is:
(i) C₆-C₁₀ aryl, which is optionally substituted with from 1-3 independently selected R^{c}; or
(ii) heteroaryl containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{c}; or
(iii) C₁-C₆ alkyl or C₁-C₆ haloalkyl, each of which is optionally substituted with a substituent selected from —OH and —CN;
R⁶ is C₁-C₆ alkyl 1 or C₁-C₆ haloalkyl, each of which is optionally substituted with a substituent selected from —OH and —CN; or

### R³ is:

(i) C₆-C₁₀ aryl, which is optionally substituted with from 1-3 independently selected R^{d}; or
(ii) heteroaryl, each containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{d};
R^{a} at each occurrence is, independently, selected from halo, —OH, C₁-C₆ alkoxy, C₁-C₆ thioalkoxy, C₁-C₆ haloalkoxy, C₁-C₆ thiohaloalkoxy, C₁-C₆ alkyl, C₁-C₆ haloalkyl, and —CN;
R^{b} at each occurrence is, independently selected from halo, —OH, C₁-C₆ alkoxy, C₁-C₆ thioalkoxy, C₁-C₆ haloalkoxy, C₁-C₆ thiohaloalkoxy, C₁-C₆ alkyl, C₁-C₆ haloalkyl, —NH₂, —NH(C₁-C₆ alkyl), N(C₁-C₆ alkyl)₂, —NHC(O)(C₁-C₆ alkyl), —CN; and —NO₂;
R^{c} at each occurrence is independently selected from the substituents delineated in (aa), (bb) and (cc) below:
(aa) halo; C₁-C₆ alkoxy; C₁-C₆ haloalkoxy; C₁-C₆ thioalkoxy; C₁-C₆ thiohaloalkoxy; C₁-C₆ alkyl, C₁-C₆ haloalkyl, —NH(C₁-C₆ alkyl), N(C₁-C₆ alkyl)₂, —NHC(O)(C₁-C₆ alkyl), wherein the alkyl portion of each is optionally substituted with —OH;
(bb)-OH; —CN; nitro; —NH₂; azido; C₂-C₄ alkenyl; C₂-C₄ alkynyl; —C(O)H; —C(O)(C₁-C₆ alkyl); C(O)OH; —C(O)O(C₁-C₆ alkyl); —C(O)NH₂—SO₂(C₁-C₆ alkyl); —SO₂(C₁-C₆ haloalkyl); —C(O)NR′″R″″, —SO₂NR′″R″″, —SO₂NH₂, —NHCO(C₁-C₆ alkyl), —NHSO₂(C₁-C₆ alkyl), whereby R′″ and R″″ is independently selected from H, C₁-C₆ alkyl, C₁-C₆ haloalkyl.
(cc) C₃-C₆ cycloalkyl or heterocyclyl containing from 5-6 ring atoms, wherein from 1-2 of the ring atoms of the heterocyclyl is independently selected from N, NH, N(C₁-C₆ alkyl), NC(O)(C₁-C₆ alkyl), O, and S; and wherein each of said cycloalkyl and heterocyclyl is optionally substituted with from 1-3 independently selected C₁-C₄ alkyl groups;
   and
   R^{d} at each occurrence is, independently selected from halo, C₁-C₆ alkoxy, C₁-C₆ thioalkoxy, C₁-C₆ haloalkoxy, C₁-C₆ thiohaloalkoxy, C₁-C₆ alkyl, C₁-C₆ haloalkyl, and —CN; COOH, NO₂, C(O)(C₁-C₆ alkyl), C(O)(C₁-C₆ haloalkyl), azido, NCS, —CH₂OH, amino, NR′″R″″, N-azidinyl, N-morpholinyl, S(C₁-C₆ alkyl), —SO₂(C₁-C₆ alkyl), —C(O)NR′″R″″—SO₂NR′″R″″, —SO₂NH₂, —NHCO(C₁-C₆ alkyl), —NHSO₂(C₁-C₆ alkyl), whereby R′″ and R″″ is independently selected from H, C₁-C₆ alkyl, C₁-C₆ haloalkyl;
   or a pharmaceutically acceptable salt thereof.

In some embodiments, it is provided that when R² is substituted with —OH, then A-R¹ is not 2,4-difluorobenzyl or 4-methoxybenzyl.

[B] In some embodiments:

### R¹ is:

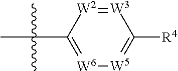

wherein:
W², W³, W⁵, and W⁶ are defined according to (A) or (B) below:
(A) each of W², W³, W⁵, and W⁶ is independently selected from CH, C(halo), or COR (where R═H, C₁-C₆ alkyl); or
(B) one or two of W², W³, W⁵, and W⁶ are N; and the others are independently selected from CH or C(halo);
R⁴ is selected from:
(i) halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); —NHC(O)R⁴¹, —NHSO₂R⁴¹, —SO₂N(R⁴²)(R⁴³); —C(O)NHCH(CH₂OH)₂, —C(O)NH(CH₂)₃COOH,
(ii) C₁-C₆ alkoxy, OCH(CH₂OH)₂, C₁-C₆ thioalkoxy, C₁-C₆ haloalkoxy, C₁-C₆ halothioalkoxy, C₁-C₆ alkyl, C₁-C₆ haloalkyl, each of which is optionally substituted with from 1-3 (e.g., 1-2 or 1) substituents independently selected from —OH and —CN;
(iii) heterocyclyl, containing from 3-8 ring atoms, wherein from 1-2 of the ring atoms is independently selected from N, NH, N(C₁-C₆ alkyl), O, and S; and wherein said heterocyclic ring is optionally substituted with from 1-3 independently selected R^{a};
(iv) heterocycloalkenyl or heteroaryl, each containing 5 ring atoms, wherein from 1-4 of the ring atoms is independently selected from N, NH, N(C₁-C₆ alkyl), O, and S; and wherein said heteroaryl ring is optionally substituted with from 1-3 independently selected le; and
(v) hydrogen;
R⁴¹ is C₁-C₈ alkyl, C₁-C₈ haloalkyl, or benzyl optionally substituted with from 1-3 R^{b};
each of R⁴² and R⁴³ is independently selected from hydrogen; C₁-C₈ alkyl or C₁-C₈ haloalkyl, each of which is optionally substituted with from 1-3 substituents independently selected from —OH; OCH₃, CN, COOH and —NHC(O)(C₁-C₃ alkyl);
R⁴⁴ is hydrogen, C₁-C₈ alkyl, or C₁-C₈ haloalkyl;
R⁴⁵ is C₁-C₈ alkyl or C₁-C₈ haloalkyl;
   A is C(R^{A})₂, wherein each occurrence of R^{A} is independently selected from hydrogen and —CH₃;

### R² is:

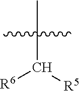 [0055] R⁵ is:
(i) C₆-C₁₀ aryl, which is optionally substituted with from 1-3 independently selected R^{c}; or
(ii) heteroaryl containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{c}; or
(iii) C₁-C₆ alkyl or C₁-C₆ haloalkyl, each of which is optionally substituted with a substituent selected from —OH and —CN;
R⁶ is C₁-C₆ alkyl or C₁-C₆ haloalkyl, each of which is optionally substituted with a substituent selected from —OH and —CN; or

### R³ is:

(i) C₆-C₁₀ aryl, which is optionally substituted with from 1-3 independently selected R^{d}; or
(ii) heteroaryl, each containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{d};
R^{a} at each occurrence is, independently, selected from halo, —OH, C₁-C₆ alkoxy, C₁-C₆ thioalkoxy, C₁-C₆ haloalkoxy, C₁-C₆ thiohaloalkoxy, C₁-C₆ alkyl, C₁-C₆ haloalkyl, and —CN;
R^{b} at each occurrence is, independently selected from halo, —OH, C₁-C₆ alkoxy, C₁-C₆ thioalkoxy, C₁-C₆ haloalkoxy, C₁-C₆ thiohaloalkoxy, C₁-C₆ alkyl, C₁-C₆ haloalkyl, —NH₂, —NH(C₁-C₆ alkyl), N(C₁-C₆ alkyl)₂, —NHC(O)(C₁-C₆ alkyl), —CN; and —NO₂;
R^{c} at each occurrence is independently selected from the substituents delineated in (aa), (bb) and (cc) below:
(aa) halo; C₁-C₆ alkoxy; C₁-C₆ haloalkoxy; C₁-C₆ thioalkoxy; C₁-C₆ thiohaloalkoxy; C₁-C₆ alkyl, C₁-C₆ haloalkyl, —NH(C₁-C₆ alkyl), N(C₁-C₆ alkyl)₂, —NHC(O)(C₁-C₆ alkyl), wherein the alkyl portion of each is optionally substituted with —OH;
(bb) —OH; —CN; nitro; —NH₂; azido; C₂-C₄ alkenyl; C₂-C₄ alkynyl; —C(O)H; —C(O)(C₁-C₆ alkyl); C(O)OH; —C(O)O(C₁-C₆ alkyl); —C(O)NH₂—SO₂(C₁-C₆ alkyl); —SO₂(C₁-C₆ haloalkyl); —C(O)NR′″R″″—SO₂NR′″R″″, —SO₂NH₂, —NHCO(C₁-C₆ alkyl), —NHSO₂(C₁-C₆ alkyl), whereby R′″ and R″″ is independently selected from H, C₁-C₆ alkyl, C₁-C₆ haloalkyl.
(cc) C₃-C₆ cycloalkyl or heterocyclyl containing from 5-6 ring atoms, wherein from 1-2 of the ring atoms of the heterocyclyl is independently selected from N, NH, N(C₁-C₆ alkyl), NC(O)(C₁-C₆ alkyl), O, and S; and wherein each of said cycloalkyl and heterocyclyl is optionally substituted with from 1-3 independently selected C₁-C₄ alkyl groups;
   and
   R^{d} at each occurrence is, independently selected from halo, C₁-C₆ alkoxy, C₁-C₆ thioalkoxy, C₁-C₆ haloalkoxy, C₁-C₆ thiohaloalkoxy, C₁-C₆ alkyl, C₁-C₆ haloalkyl, and —CN; COOH, NO₂, C(O)(C₁-C₆ alkyl), C(O)(C₁-C₆ haloalkyl), azido, NCS, —CH₂OH, amino, NR′″R″″, N-azidinyl, N-morpholinyl, S(C₁-C₆ alkyl), —SO₂(C₁-C₆ alkyl), —C(O)NR′″R″″—SO₂NR′″R″″, —SO₂NH₂, —NHCO(C₁-C₆ alkyl), —NHSO₂(C₁-C₆ alkyl), whereby R′″ and R″″ is independently selected from H, C₁-C₆ alkyl, C₁-C₆ haloalkyl or a pharmaceutically acceptable salt thereof.

In some embodiments, it is provided that when R² is substituted with —OH, then A-R¹ is not 2,4-difluorobenzyl or 4-methoxybenzyl.

[C] In some embodiments:

### R¹ is:

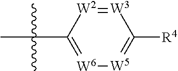

wherein:
W², W³, W⁵, and W⁶ are defined according to (A) or (B) below:

### A

each of W² and W⁶ is independently selected from CH and C(halo); and
each of W³ and W⁵ is independently selected from CH, C(halo), and CR′; wherein R′ is —C(O)OH, —C(O)O(C₁-C₆ alkyl), or —CN; or

### B

one or two of W², W³, W⁵, and W⁶ are N; and the others are independently selected from CH and C(halo);
R⁴ is selected from any of the substituents delineated in (i)-(v) immediately below:
(i) halo; —CO₂H; —C(O)OR⁴¹; —NHC(O)OR⁴¹; —N(CH₃)C(O)OR⁴¹;
—C(O)N(R⁴²)(R⁴³); —C(O)R⁴⁴; —CN; —NO₂; —SO₃H; —P(O)(OH)₂; —OH, —SO₂(R⁴⁵); —NHC(O)R⁴¹, —NHSO₂R⁴¹, —SO₂N(R⁴²)(R⁴³); —C(O)NHCH(CH₂OH)₂, —C(O)NH(CH₂)₃COOH; OCH(CH₂OH)₂;
(ii) C₁-C₆ alkoxy, C₁-C₆ thioalkoxy, C₁-C₆ haloalkoxy, C₁-C₆ halothioalkoxy, C₁-C₆ alkyl, C₁-C₆ haloalkyl, each of which is optionally substituted with from 1-3 (e.g., 1-2 or 1) substituents independently selected from —OH, C₁-C₃ alkoxy, —C(O)OH, —C(O)O(C₁-C₆ alkyl), and —CN;
(iii) heterocyclyl or heterocyclyloxy, each containing from 3-8 ring atoms, wherein from 1-2 of the ring atoms is independently selected from N, NH, N(C₁-C₆ alkyl), O, and S; and wherein said heterocyclyl or heterocyclyloxy is optionally substituted with from 1-3 independently selected R^{a};
(iv) heterocycloalkenyl or heteroaryl, each containing 5 ring atoms, wherein from 1-4 of the ring atoms is independently selected from N, NH, N(C₁-C₆ alkyl), O, and S; and wherein said heteroaryl ring is optionally substituted with from 1-3 independently selected R^{b}; and
(v) hydrogen;
R⁴¹ is C₁-C₈ alkyl, C₁-C₈ haloalkyl, or benzyl optionally substituted with from 1-3 R^{b};
each of R⁴² and R⁴³ is, independently:
(i) hydrogen; or
(ii) C₁-C₈ alkyl; C₁-C₈ haloalkyl; C₃-C₈ cycloalkyl; and heterocyclyl containing from 3-8 ring atoms, wherein from 1-2 of the ring atoms is independently selected from N, NH, N(C₁-C₆ alkyl), O, and S; and wherein each of said alkyl, haloalkyl, cycloalkyl, and heterocyclyl is optionally substituted with from 1-3 R^{C};
or
R⁴²—N—R⁴³ together forms a saturated ring having 5 or 6 ring atoms, in which from 1 or 2 of said ring atoms, in addition to the N that occurs between R⁴² and R⁴³, is/are optionally a heteroatom independently selected from NH, N(alkyl), O, or S; and wherein said saturated ring is optionally substituted with from 1-3 R^{C};
R⁴⁴ is hydrogen, C₁-C₈ alkyl, or C₁-C₈ haloalkyl;
R⁴⁵ is C₁-C₈ alkyl or C₁-C₈ haloalkyl;
   in embodiments, it is provided that only one of R⁴ and R′ or only one of R⁴ and two occurrences of R′ can be —C(O)OH, —C(O)O(C₁-C₆ alkyl), or —CN;
   A is C(R^{A})₂, wherein each occurrence of R^{A} is independently selected from hydrogen and —CH₃;

### R² is:

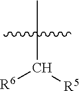 [0087] R⁵ is:
(i) C₆-C₁₀ aryl, which is optionally substituted with from 1-3 independently selected R^{c}; or
(ii) heteroaryl containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{c}; or
(iii) C₁-C₆ alkyl or C₁-C₆ haloalkyl, each of which is optionally substituted with a substituent selected from —OH and —CN;
R⁶ is C₁-C₆ alkyl or C₁-C₆ haloalkyl, each of which is optionally substituted with a substituent selected from —OH and —CN; or

### R³ is:

(i) C₆-C₁₀ aryl, which is optionally substituted with from 1-3 independently selected R^{d};
or
(ii) heteroaryl, each containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{d};
R^{a} at each occurrence is, independently, selected from halo, —OH, C₁-C₆ alkoxy, C₁-C₆ thioalkoxy, C₁-C₆ haloalkoxy, C₁-C₆ thiohaloalkoxy, C₁-C₆ alkyl, C₁-C₆ haloalkyl, and —CN;
R^{b} at each occurrence is, independently selected from halo, —OH, C₁-C₆ alkoxy, C₁-C₆ thioalkoxy, C₁-C₆ haloalkoxy, C₁-C₆ thiohaloalkoxy, C₁-C₆ alkyl, C₁-C₆ haloalkyl, —NH₂, —NH(C₁-C₆ alkyl), N(C₁-C₆ alkyl)₂, —NHC(O)(C₁-C₆ alkyl), —CN; and —NO₂;
R^{C} at each occurrence is independently selected from the substituents delineated in (aa), (bb) and (cc) below:
(aa) halo; C₁-C₆ alkoxy; C₁-C₆ haloalkoxy; C₁-C₆ thioalkoxy; C₁-C₆ thiohaloalkoxy; C₁-C₆ alkyl, C₁-C₆ haloalkyl, —NH(C₁-C₆ alkyl), N(C₁-C₆ alkyl)₂, —NHC(O)(C₁-C₆ alkyl), wherein the alkyl portion of each is optionally substituted with —OH, C₁-C₃ alkoxy, —C(O)OH, —C(O)O(C₁-C₆ alkyl), and —CN;
(bb) —OH; —CN; nitro; —NH₂; azido; C₂-C₄ alkenyl; C₂-C₄ alkynyl; —C(O)H; —C(O)(C₁-C₆ alkyl); C(O)OH; —C(O)O(C₁-C₆ alkyl); —C(O)NH₂—SO₂(C₁-C₆ alkyl); —SO₂(C₁-C₆ haloalkyl); —C(O)NR′″R″″—SO₂NR′″R″″, —SO₂NH₂, —NHCO(C₁-C₆ alkyl), —NHSO₂(C₁-C₆ alkyl), whereby R′″ and R″″ is independently selected from H, C₁-C₆ alkyl, C₁-C₆ haloalkyl.
(cc) C₃-C₆ cycloalkyl or heterocyclyl containing from 5-6 ring atoms, wherein from 1-2 of the ring atoms of the heterocyclyl is independently selected from N, NH, N(C₁-C₆ alkyl), NC(O)(C₁-C₆ alkyl), O, and S; and wherein each of said cycloalkyl and heterocyclyl is optionally substituted with from 1-3 independently selected C₁-C₄ alkyl groups;
   and
   R^{d} at each occurrence is, independently selected from halo, C₁-C₆ alkoxy, C₁-C₆ thioalkoxy, C₁-C₆ haloalkoxy, C₁-C₆ thiohaloalkoxy, C₁-C₆ alkyl, C₁-C₆ haloalkyl, and —CN; COOH, NO₂, C(O)(C₁-C₆ alkyl), C(O)(C₁-C₆ haloalkyl), azido, NCS, —CH₂OH, amino, NR′″R″″, N-azidinyl, N-morpholinyl, S(C₁-C₆ alkyl), —SO₂(C₁-C₆ alkyl), —C(O)NR′″R″″—SO₂NR′″R″″, —SO₂NH₂, —NHCO(C₁-C₆ alkyl), —NHSO₂(C₁-C₆ alkyl), whereby R′″ and R″″ is independently selected from H, C₁-C₆ alkyl, C₁-C₆ haloalkyl; or a pharmaceutically acceptable salt thereof.

In some embodiments, it is provided that when R² is substituted with —OH, then A-R¹ is not 2,4-difluorobenzyl or 4-methoxybenzyl.

In some embodiments, it is provided that when R² is substituted with (one or more) —OH, then R⁴ cannot be hydrogen, halo, or C₁-C₆ alkoxy, except that when R² is unsubstituted alkyl or alkyl that is substituted with one or more —OH, then R⁴ can be C₁-C₆ alkoxy when either R′ is —C(O)OH, —C(O)O(C₁-C₆ alkyl); or when two or more of W², W³, W⁵, and W⁶ are each independently C(halo).

In some embodiments, it is provided that when R² is unsubstituted alkyl or alkyl that is substituted with one or more —OH, then R⁴ cannot be hydrogen, halo, or C₁-C₆ alkoxy, except that when R² is unsubstituted alkyl or alkyl that is substituted with one or more —OH, then R⁴ can be C₁-C₆ alkoxy when either R′ is —C(O)OH, —C(O)O(C₁-C₆ alkyl); or when two or more of W², W³, W⁵, and W⁶ are each independently C(halo).

In another aspect, any of the formula (I) compounds specifically described herein are featured.

[II] In one aspect, compositions (e.g., a pharmaceutical composition) are featured which includes a compound of formula (I) (including any subgenera or specific compound thereof as described anywhere herein, including those in the claims) or a salt (e.g., a pharmaceutically acceptable salt) thereof as defined anywhere herein and a pharmaceutically acceptable carrier. In some embodiments, the compositions include an effective amount of the compound or salt. In some embodiments, the compositions can further include one or more additional therapeutic agents.

[III] In one aspect, methods are featured for treating (e.g., controlling, relieving, ameliorating, alleviating, or slowing the progression of) or for preventing (e.g., delaying the onset of or reducing the risk of developing) a disease, disorder, or condition associated with γ-secretase activity. The methods include administering to a subject having (or at risk of having) the disease, disorder, or condition a therapeutically effective amount of a compound of formula (I) (including any subgenera or specific compound thereof as described anywhere herein, including those in the claims) or a salt (e.g., a pharmaceutically acceptable salt) thereof as defined anywhere herein, or a therapeutic preparation, composition, or formulation thereof.

In certain embodiments, the disease, disorder, or condition can be. a neurodegenerative disorder, e.g., Alzheimer's disease.

In other embodiments, the subject can be a subject that has, or is at risk of developing, cancer. The cancer can be a gastrointestinal cancer (e.g., cancer of the esophagus, gallbladder, liver, pancreas, stomach, small intestine, large intestine, colon, or rectum). In some embodiments, the cancer can be leukemia or any solid tumors of which inhibition of γ-secretase can lead to therapeutic effects in cancer chemotherapy.

It should be appreciated that any one or more of the compounds of formula (I) may be used to inhibit γ-secretase activity by interaction with γ-secretase (e.g., in vitro or in vivo) with any one or more of the compounds. The invention also relates to methods of making medicaments for use in treating a subject, e.g., for treating a subject having a disease, disorder, or condition associated with γ-secretase activity, or at risk of developing disease, disorder, or condition associated with γ-secretase activity, treating a subject having Alzheimer's disease, or at risk of developing Alzheimer's disease, inhibiting APP cleavage, and/or inhibiting γ-secretase activity. Accordingly, one or more compounds or compositions described herein that inhibit γ-secretase activity as described herein may be used for the preparation of a medicament for use in any of the methods of treatment described herein. In some embodiments, the invention provides for the use of one or more compounds or compositions of the invention for the manufacture of a medicament or pharmaceutical for treating a mammal (e.g., a human) having one or more symptoms of, or at risk for, a disease or condition associated with γ-secretase activity (e.g., Alzheimer's disease).

In some embodiments, a compound of formula (I) (including any subgenera or specific compound thereof as described anywhere herein, including those in the claims) or a salt (e.g., a pharmaceutically acceptable salt) thereof as defined anywhere herein inhibits γ-secretase activity by at least 10% (e.g., by about 50%, by about 75%, by about 80%, by about 90%, by about 95%, or more, for example, completely inhibits) at a concentration of 1, 10 or 100 μM in an assay described herein (e.g., the γ-secretase assay). Accordingly, in some embodiments, a compound of the invention does not have less than 10% inhibitory activity when assayed at a concentration of about 1, 10 or 100 μM in an assay described herein (e.g., γ-secretase assay). In some embodiments, the inhibitory activity of a compound is selective for γ-secretase mediated cleavage of APP relative to the Notch protein. Accordingly, in some embodiments, a compound of the invention inhibits γ-secretase activity against APP (e.g., by at least 10%, by about 50%, by about 75%, by about 80%, by about 90%, by about 95%, or more, for example, completely inhibits) to a greater extent than it inhibits γ-secretase activity against the Notch protein. In some embodiments, a compound of the invention that inhibits APP cleavage does not inhibit Notch cleavage significantly (e.g., no inhibition of Notch cleavage, or enhanced Notch cleavage, is observed using an assay described herein, for example the N-100 assay or other assay). In some embodiments, an inhibitor is at least 5 fold (e.g., at least 10 fold, at least 100 fold, etc.) more selective for inhibiting APP cleavage relative to Notch cleavage. In certain embodiments, a compound of the invention has an IC₅₀ value of from about 28 nM to about 13 μM for APP (Aβ1-40) in the in vitro biochemical assay but a higher IC₅₀ value (e.g., from about 8 μM to about 30 μM) for Notch in the N-100assay. In other embodiments, in cellular assays, a compound of the invention has an IC₅₀ value of from about 15 nM to about 500 nM for APP (Aβ40) and an IC₅₀ value of from about 1 nM to 100 nM for APP (Aβ42) was observed and a higher IC₅₀ value (e.g., 34 μM) as determined in a Notch cellular assay. However, it should be appreciated that a compound of the invention may be selective even if it has a higher IC₅₀ value for APP, provided that the IC₅₀ value for Notch is relatively higher.

In some embodiments, the subject can be in need thereof (e.g., a subject identified as being in need of such treatment, such as a subject having, or at risk of having, one or more of the diseases or conditions described herein). Identifying a subject in need of such treatment can be in the judgment of a subject or a health care professional and can be subjective (e.g., opinion) or objective (e.g., measurable by a test or diagnostic method). In some embodiments, the subject can be a mammal. In certain embodiments, the subject can be a human.

In some embodiments, abnormally high levels of γ-secretase activity imply statistically significantly higher levels (e.g., 10% higher, 20% higher, 30% higher, 50% higher, or higher) than a reference level characteristic of normal levels of activity.

However, it should be appreciated that AD patients or those at risk of developing AD may not necessarily have elevated levels of γ-secretase and/or elevated γ-secretase activity. Instead such subjects may suffer the effects of Aβ which is pathogenic and which can be produced by γ-secretase at all levels. In some embodiments, elevated levels of Aβ are pathogenic. Levels of Aβ depend on a balance between production and clearance. There are many factors that are involved in the production and clearance of Aβ. Accordingly, in some embodiments decreasing the γ-secretase-mediated production of Aβ can slow, halt and/or prevent the neurodegenerative effects of Aβ. Therefore, decreasing the γ-secretase production of Aβ (by up to 10%, or up to 20%, or up to 30%, or up to 40%, or up to 50%, or higher) relative to a baseline activity can yield a therapeutic effect and/or prevent disease onset and/or delay the onset of AD. It should be appreciated that γ-secretase activity in a subject can be measured from Aβ levels in plasma and cerebral spinal fluid (CSF). Accordingly, levels of Aβ inhibition can be assayed by measuring Aβ levels in the plasma and CSF with different compounds and comparing the levels to a reference level obtained without a test compound or using a compound that is known not to affect Aβ inhibition (e.g., a reference compound that is not a γ-secretase inhibitor). In some embodiments, compositions of the invention are administered to a patient that has, or is at risk of developing, Alzheimer's disease.

The term “subject having (or at risk of having) neurodegenerative disorders” (and the like) refers to a subject that is affected by or at risk of developing neurodegenerative disorders (e.g. predisposed, for example, genetically predisposed, to developing Alzheimer's disease) and/or any neurodegenerative disorders characterized by pathological aggregations of β-amyloid proteins or peptide fragments.

[IV] In one aspect, methods of making the pharmaceutical compositions described herein are featured. In embodiments, the methods include taking any one or more of the compounds of formula (I) (including any subgenera or specific compound thereof as described anywhere herein, including those in the claims) or a salt (e.g., a pharmaceutically acceptable salt) thereof as defined anywhere herein, and mixing said compound(s) with one or more pharmaceutically acceptable carriers.

[V] In one aspect, kits for treating (e.g., controlling, relieving, ameliorating, alleviating, or slowing the progression of) or for preventing (e.g., delaying the onset of or reducing the risk of developing) a disease, disorder, or condition associated with γ-secretase activity, e.g., a neurodegenerative disorder, e.g., Alzheimer's disease, in a subject are featured. The kits include (i) a compound of formula (I) (including any subgenera or specific compound thereof as described anywhere herein, including those in the claims) or a salt (e.g., a pharmaceutically acceptable salt) thereof as defined anywhere herein; and (ii) instructions that include a direction to administer said compound to a subject (e.g., a patient). In a preferred embodiment the subject is a human. In some embodiments, an article of manufacture may include two or more compounds or compositions of the invention alone or along with one or more additional compounds or compositions that are useful for treating Alzheimer's disease as described herein.

[VI] In another aspect, methods of making the compounds described herein are featured. In embodiments, the methods include taking any one of the intermediate compounds described herein and reacting it with one or more chemical reagents in one or more steps to produce a compound of formula (I) (including any subgenera or specific compound thereof as described anywhere herein, including those in the claims) or a salt (e.g., a pharmaceutically acceptable salt) thereof as defined anywhere herein.

[VII] In embodiments, any compound, composition, or method described herein can also include any one or more of the other features delineated in the detailed description and/or in the claims. For example, embodiments can include one or more of the following features delineated below.

Each of W², W³, W⁵, and W⁶ is independently selected from CH or C(halo).

Each of W², W³, W⁵, and W⁶ is CH.

One or two of W², W³, W⁵, and W⁶ is N; and the others are independently selected from CH or C(halo). For example, each of W³ and W⁵ is N; and W² is CH and W⁶ is C(halo). As another example, one of W² and W³ is N; and the others are independently selected from CH or C(halo).

W², W³, W⁵, and W⁶ are defined according to definition (A).

Each of W³ and W⁵ is independently selected from CH and C(halo). For example, each of W², W³, W⁵, and W⁶ is CH.

One of W³ and W⁵ is CR′, and the other of W³ and W⁵ is CH or C(halo) (e.g., CH). In embodiments, each of W² and W⁶ is CH. In embodiments, R′ is —C(O)OH or —C(O)O(C₁-C₆ alkyl) (e.g., —C(O)OH).

W², W³, W⁵, and W⁶ are defined according to definition (B). In embodiments, one or two of W³ and W⁵ is/are N. For example, one of W³ and W⁵ is N; the other of W³ and W⁵ is CH; and each of W² and W⁶ is CH.

R⁴ is selected from halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, C₁-C₆ alkoxy, and —SO₂(R⁴⁵).

R⁴ is selected from —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; and —SO₂(R⁴⁵).

R⁴ is selected from halo; —CO₂H; —C(O)OR⁴¹; —NHC(O)OR⁴¹; —N(CH₃)C(O)OR⁴¹;

—C(O)N(R⁴²)(R⁴³); —C(O)R⁴⁴; —CN; —NO₂; —SO₃H; —P(O)(OH)₂; —OH, and —SO₂(R⁴⁵).

R⁴ is selected from —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; and —SO₂(R⁴⁵).

R⁴ is —CO₂H.

R⁴ is —SO₂ (R⁴⁵). In embodiments, R⁴⁵ is C₁-C₈ alkyl (e.g., —CH₃).

R⁴ is —C(O)N(R⁴²)(R⁴³).

In embodiments, each of R⁴² and R⁴³ is independently selected from:
(i) hydrogen;
(ii) C₁-C₈ alkyl; C₁-C₈ haloalkyl; C₃-C₈ cycloalkyl; and heterocyclyl containing from 3-8 ring atoms, wherein from 1-2 of the ring atoms is independently selected from N, NH, N(C₁-C₆ alkyl), O, and S; and wherein each of said alkyl, haloalkyl, cycloalkyl, and heterocyclyl is optionally substituted with from 1-3 (e.g., 1) R^{c}.

One of R⁴² and R⁴³ is hydrogen; and the other of R⁴² and R⁴³ is C₁-C₈ alkyl; C₁-C₈ haloalkyl; C₃-C₈ cycloalkyl; and heterocyclyl containing from 3-8 (e.g., 3-6, 5-6) ring atoms, wherein from 1-2 of the ring atoms is independently selected from N, NH, N(C₁-C₆ alkyl), O, and S; and wherein each of said alkyl, haloalkyl, cycloalkyl, and heterocyclyl is optionally substituted with from 1-3 (e.g., 1) R^{c}.

In embodiments, one of R⁴² and R⁴³ is hydrogen; and the other of R⁴² and R⁴³ is C₁-C₈ alkyl, which is optionally substituted with from 1-3 (e.g., 1) R^{c}.

In embodiments, R^{c} at each occurrence is, independently, —OH; C₁-C₆ alkoxy (e.g., OCH₃); —C(O)(C₁-C₆ alkyl) (e.g., —C(O)CH₃); or heterocyclyl (e.g., pyranyl, e.g., 4-pyranyl) containing from 5-6 ring atoms, wherein from 1-2 of the ring atoms of the heterocyclyl is independently selected from N, NH, N(C₁-C₆ alkyl), NC(O)(C₁-C₆ alkyl), O, and S; and wherein said heterocyclyl is optionally substituted with from 1-3 substituents independently selected from —OH and C₁-C₄ alkyl.

For example, R⁴ is selected from —C(O)NHCH(CH₂OH)₂, OCH(CH₂OH)₂.

One of R⁴² and R⁴³ is hydrogen; and the other of R⁴² and R⁴³ is C₃-C₈ cycloalkyl; or heterocyclyl containing from 3-8 ring atoms, wherein from 1-2 of the ring atoms is independently selected from N, NH, N(C₁-C₆ alkyl), O, and S; and wherein each of said cycloalkyl or heterocyclyl is optionally substituted with from 1-3 (e.g., 1) R^{c} (e.g., —OH).

R⁴²—N—R⁴³ together forms a saturated ring having 5 or 6 ring atoms, in which from 1 or 2 ring atoms, in addition to the N that occurs between R⁴² and R⁴³, is/are optionally a heteroatom independently selected from NH, N(alkyl), O, or S; and wherein said saturated ring is optionally substituted with from 1-3 R^{c} (e.g., R⁴²—N—R⁴³ together forms a morpholino ring)

R⁴ is heterocyclyloxy, each containing from 3-8 ring atoms, wherein from 1-2 of the ring atoms is independently selected from N, NH, N(C₁-C₆ alkyl), O, and S; and wherein said heterocyclyloxy is optionally substituted with from 1-3 independently selected R^{a} (e.g., pyranyloxy).

R⁴ is heterocyclyl, each containing from 3-8 ring atoms, wherein from 1-2 of the ring atoms is independently selected from N, NH, N(C₁-C₆ alkyl), O, and S; and wherein said heterocyclic ring is optionally substituted with from 1-3 independently selected R^{a}.

Each of W², W³, W⁵, and W⁶ is independently selected from CH or C(halo); and

R⁴ is selected from:
(i) halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); and
(iii) heterocyclyl or each containing from 3-8 ring atoms, wherein from 1-2 of the ring atoms is independently selected from N, NH, N(C₁-C₆ alkyl), O, and S; and wherein said heterocyclic ring is optionally substituted with from 1-3 independently selected R^{a}.

In certain embodiments, one or more of the following can apply. Each of W², W³, W⁵, and W⁶ is CH. R⁴ is selected from —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; and —SO₂(R⁴⁵). For example, R⁴ can be —CO₂H. As another example, R⁴ is —SO₂(R⁴⁵), and in embodiments, R⁴⁵ can be C₁-C₈ alkyl (e.g., —CH₃). R⁴ can be —C(O)N(R⁴²)(R⁴³).

R⁵ is:
(i) C₆-C₁₀ aryl, which is optionally substituted with from 1-3 independently selected R^{c}; or
(ii) heteroaryl containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{c}.

In certain embodiments, R⁵ is C₆-C₁₀ aryl, which is optionally substituted with from 1-3 independently selected R^{c}. For example, R⁵ can be phenyl, which is optionally substituted with from 1-3 independently selected R^{c} (e.g., unsubstituted phenyl).

R⁶ is C₁-C₆ alkyl, which is optionally substituted with a substituent selected from —OH and —CN (e.g., —OH). For example, can be —CH₂CH₃ or —CH₃.

In certain embodiments:

R⁵ is C₆-C₁₀ aryl, which is optionally substituted with from 1-3 independently selected R^{c}; and

R⁶ is C₁-C₆ alkyl, which is optionally substituted with a substituent selected from —OH and —CN (e.g., —OH).

In certain embodiments, one or more of the following can apply. R⁵ is phenyl, which is optionally substituted with from 1-3 independently selected R^{e} (e.g., unsubstituted phenyl). R⁶ is —CH₂CH₃ or —CH₃.

The carbon attached to R⁵ and R⁶ has the S configuration.

R³ is C₆-C₁₀ aryl, which is optionally substituted with from 1-3 independently selected R^{d}. In embodiments, R³ is phenyl that is substituted with 1 or 2 independently selected R^{d}. For example, R³ can be 4-chloro-phenyl, 4-fluoro-phenyl, or 2,4-difluorophenyl.

R³ is heteroaryl containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{d}. In embodiments, R³ is heteroaryl containing from 5-6 ring atoms, wherein from 1-4 of the ring atoms is independently selected from N, NH, N(C₁-C₆ alkyl), O, and S; and wherein said heteroaryl ring is substituted with 1 or 2 independently selected R^{d}. For example, R³ can be thienyl, which is substituted with 1 or 2 independently selected R^{d}.

R^{d} at each occurrence is independently selected from halo.

A is CH₂.

[VIII] Embodiments can include any one or more of the following advantages. Some of the compounds of formula (I) selectively inhibit γ-secretase-mediated cleavage of APP with little or no inhibition of the γ-secretase-mediated cleavage of the Notch family of transmembrane receptors. Selective inhibition of the cleavage of APP relative to that of the Notch receptor is believed to minimize certain unwanted side effects, such as lymphopoiesis and intestinal cell differentiation. For example, in an in vivo efficacy study at 100 mg/kg b.i.d. for 7 consecutive days no toxicity was observed in the transgenic and nontransgenic mice employed in the study using one of the claimed compounds in this invention (e.g., Example 1). This is an indication that there could be a minimization of side effects with these types of compounds

Some of the compounds of formula (I) exhibit enhanced solubility in aqueous media. For example, some of the compounds of formula (I) (e.g., compounds in which R⁴ is other than hydrogen, e.g., compounds in which R⁴ is C(O)OH) exhibit a solubility that is 285 μM in a PBS buffer at pH 7.4. In embodiments, the compounds described herein exhibited a range of solubility from about 0.17 μM to about 280 μM in PBS at pH 7.4

Some of the compounds of formula (I) exhibit enhanced metabolic stability. For example, some of the compounds of formula (I) (e.g., compounds in which R⁴ is C(O)OH or SO₂CH₃) exhibited enhanced metabolic stability (e.g., greater than about 90% of test compound remaining after 60 minutes) when exposed to human liver microsomes with or without NADPH.

Some of the compounds of formula (I) exhibit reduced) intrinsic clearance. For example, some of the compounds of formula (I) (e.g., compounds in which R⁴ is C(O)OH or SO₂CH₃) exhibited reduced intrinsic clearance (e.g., less than about 10 μL/min/mg/proteins) in human cells.

### DEFINITIONS

The term “mammal” includes organisms, which include mice, rats, cows, sheep, pigs, rabbits, goats, horses, monkeys, dogs, cats, and humans.

“An effective amount” refers to an amount of a compound that confers a therapeutic effect (e.g., treats, controls, relieves, ameliorates, alleviates, or slows the progression of); or prevents, e.g., delays the onset of or reduces the risk of developing, a disease, disorder, or condition or symptoms thereof on the treated subject. The therapeutic effect may be objective (i.e., measurable by some test or marker) or subjective (i.e., subject gives an indication of or feels an effect). For example, disease progression can be monitored by clinical observations, laboratory and neuroimaging investigations apparent to a person skilled in the art. The effective amount of any one or more compounds may be from about 10 ng/kg of body weight to about 1,000 mg/kg of body weight, and the frequency of administration may range from once a day to once a week. However, other dosage amounts and frequencies also may be used as the invention is not limited in this respect. It should be appreciated that one or more compounds and/or compositions of the invention may be used alone or in combination with one or more additional compounds or compositions to treat a subject that has Alzheimer's disease or that is at risk of developing Alzheimer's disease. In some embodiments, an additional compound may be an alternative inhibitor of β-amyloid production. In some embodiments, an additional compound can be a β-secretase inhibitor. In some embodiments, an additional compound may be a compound that is therapeutically useful for treating Alzheimer's disease or symptoms thereof (e.g., an acetyl-cholinesterase inhibitor, for example, Aricept; an anti-depressive agent, for example, rivastigmine; or any combination thereof). A combination therapy may involve combining one or more (e.g., 1, 2, 3, 4, 5, 6, 7, 8, 9, 10, or more) compounds of the invention with one or more (e.g., 1, 2, 3, 4, 5, 6, 7, 8, 9, 10, or more) additional compounds described herein. It should be appreciated that combination therapies may include compositions comprising of one or more compounds and/or administering one or more compounds in combination (e.g., together or separately, but according to a coordinated regimen, etc.). It should be appreciated that compounds or compositions of the invention may be administered in an amount effective to treat a neurological disorder such as Alzheimer's disease in a subject. In some embodiments, a treatment may prevent the onset or development of disease or disease symptoms in a subject at risk of the disease (e.g., in a subject with a family history of Alzheimer's, a subject with early symptoms of Alzheimer's, a subject of an age associated with a higher risk for Alzheimer's, a subject with any other risk factor for Alzheimer's, or a subject with any combination of two or more risk factors described herein). In some embodiments, a treatment may prevent or reduce the progression of the disease in a subject diagnosed as having Alzheimer's disease. In some embodiments, a treatment may promote disease regression. In preferred embodiments, the subject is a human.

Effective doses will also vary depending on route of administration, as well as the possibility of co-usage with other agents. A therapeutically effective amount can be an amount that is effective in a single dose or an amount that is effective as part of a multi-dose therapy, for example, an amount that is administered in two or more doses or an amount that is administered chronically.

The term “halo” or “halogen” refers to any radical of fluorine, chlorine, bromine or iodine.

In general, and unless otherwise indicated, substituent (radical) prefix names are derived from the parent hydride by either (i) replacing the “ane” in the parent hydride with the suffixes “yl,” “diyl,” “triyl,” “tetrayl,” etc.; or (ii) replacing the “e” in the parent hydride with the suffixes “yl,” “diyl,” “triyl,” “tetrayl,” etc. (Here the atom(s) with the free valence, when specified, is (are) given numbers as low as is consistent with any established numbering of the parent hydride). Accepted contracted names, e.g., adamantyl, naphthyl, anthryl, phenanthryl, furyl, pyridyl, isoquinolyl, quinolyl, and piperidyl, and trivial names, e.g., vinyl, allyl, phenyl, and thienyl are also used herein throughout. Conventional numbering/lettering systems are also adhered to for substituent numbering and the nomenclature of fused, bicyclic, tricyclic, and polycyclic rings.

The following definitions are used unless otherwise described. Specific and general values listed below for radicals, substituents, and ranges are for illustration only. They do not exclude other defined values or other values within defined ranges for the radicals and substituents. Unless otherwise indicated, alkyl, alkoxy, alkenyl, and the like denote both straight and branched groups.

The term “alkyl” refers to a saturated hydrocarbon chain that may be a straight chain or branched chain, containing the indicated number of carbon atoms. For example, C₁-C₆ alkyl indicates that the group may have from 1 to 6 (inclusive) carbon atoms in it. Any atom can be optionally substituted, e.g., by one or more substitutents. Examples of alkyl groups include, without limitation, methyl, ethyl, n-propyl, isopropyl, and tert-butyl.

The term “haloalkyl” refers to an alkyl group in which at least one hydrogen atom is replaced by halo. In some embodiments, more than one hydrogen atom (e.g., 2, 3, 4, 5, 6, 7, 8, 9, 10, 11, 12, 13, or 14) is replaced by halo. In these embodiments, the hydrogen atoms can each be replaced by the same halogen (e.g., fluoro) or the hydrogen atoms can be replaced by a combination of different halogens (e.g., fluoro and chloro). “Haloalkyl” also includes alkyl moieties in which all hydrogens have been replaced by halo (sometimes referred to herein as perhaloalkyl, e.g., perfluoroalkyl, such as trifluoromethyl). Any atom can be optionally substituted, e.g., by one or more substituents.

As referred to herein, the term “alkoxy” refers to a group of formula —O(alkyl). Alkoxy can be, for example, methoxy (—OCH₃), ethoxy, propoxy, isopropoxy, butoxy, iso-butoxy, sec-butoxy, tert-butoxy, pentoxy, 2-pentoxy, 3-pentoxy, or hexyloxy. Likewise, the term “thioalkoxy” refers to a group of formula —S(alkyl). The terms “haloalkoxy” and “thio-haloalkoxy” refer to —O(haloalkyl) and —S(haloalkyl), respectively. Finally, the term “heterocyclyloxy” refers to a group of the formula —O(heterocyclyl).

The term “alkenyl” refers to a straight or branched hydrocarbon chain containing the indicated number of carbon atoms and having one or more carbon-carbon double bonds. Any atom can be optionally substituted, e.g., by one or more substituents. Alkenyl groups can include, e.g., vinyl, allyl, 1-butenyl, and 2-hexenyl. One of the double bond carbons can optionally be the point of attachment of the alkenyl substituent.

The term “alkynyl” refers to a straight or branched hydrocarbon chain containing the indicated number of carbon atoms and having one or more carbon-carbon triple bonds. Alkynyl groups can be optionally substituted, e.g., by one or more substituents. Alkynyl groups can include groups such as ethynyl, propargyl, and 3-hexynyl. One of the triple bond carbons can optionally be the point of attachment of the alkynyl substituent.

The term “heterocyclyl” refers to a fully saturated monocyclic, bicyclic, tricyclic or other polycyclic ring system having one or more constituent heteroatom ring atoms independently selected from O, N (it is understood that one or two additional groups may be present to complete the nitrogen valence and/or form a salt), or S. The heteroatom or ring carbon can be the point of attachment of the heterocyclyl substituent to another moiety. Any atom can be optionally substituted, e.g., by one or more substituents. Heterocyclyl groups can include groups such as tetrahydrofuryl, tetrahydropyranyl, piperidyl (piperidino), piperazinyl, morpholinyl (morpholino), pyrrolinyl, and pyrrolidinyl. By way of example, the phrase “heterocyclic ring containing from 5-6 ring atoms”, wherein from 1-2 of the ring atoms is independently selected from N, NH, N(C₁-C₆ alkyl), NC(O)(C₁-C₆ alkyl), O, and S; and wherein said heterocyclic ring is optionally substituted with from 1-3 independently selected R^{a} would include (but not be limited to) tetrahydrofuryl, tetrahydropyranyl, piperidyl (piperidino), piperazinyl, morpholinyl (morpholino), pyrrolinyl, and pyrrolidinyl.

The term “heterocycloalkenyl” refers to partially unsaturated monocyclic, bicyclic, tricyclic, or other polycyclic hydrocarbon groups having one or more (e.g., 1-4) heteroatom ring atoms independently selected from O, N (it is understood that one or two additional groups may be present to complete the nitrogen valence and/or form a salt), or S. A ring carbon (e.g., saturated or unsaturated) or heteroatom can be the point of attachment of the heterocycloalkenyl substituent. Any atom can be optionally substituted, e.g., by one or more substituents. Heterocycloalkenyl groups can include groups such as dihydropyridyl, tetrahydropyridyl, dihydropyranyl, 4,5-dihydrooxazolyl, 4,5-dihydro-1H-imidazolyl, 1,2,5,6-tetrahydro-pyrimidinyl, and 5,6-dihydro-2H-[1,3]oxazinyl.

The term “cycloalkyl” refers to a fully saturated monocyclic, bicyclic, tricyclic, or other polycyclic hydrocarbon group. Any atom can be optionally substituted, e.g., by one or more substituents. A ring carbon serves as the point of attachment of a cycloalkyl group to another moiety. Cycloalkyl moieties can include groups such as cyclopropyl, cyclobutyl, cyclopentyl, cyclohexyl, cycloheptyl, adamantyl, and norbornyl (bicyclo[2.2.1] heptyl).

The term “aryl” refers to an aromatic monocyclic, bicyclic (2 fused rings), tricyclic (3 fused rings), or polycyclic (>3 fused rings) hydrocarbon ring system. One or more ring atoms can be optionally substituted by one or more substituents for example. Aryl moieties include groups such as phenyl and naphthyl.

The term “heteroaryl” refers to an aromatic monocyclic, bicyclic (2 fused rings), tricyclic (3 fused rings), or polycyclic (>3 fused rings) hydrocarbon group having one or more heteroatom ring atoms independently selected from O, N (it is understood that one or two additional groups may be present to complete the nitrogen valence and/or form a salt), or S. One or more ring atoms can be optionally substituted, e.g., by one or more substituents. Examples of heteroaryl groups include, but are not limited to, 2H-pyrrolyl, 3H-indolyl, 4H-quinolizinyl, benzo[b]thienyl, furyl, imidazolyl, imidizolyl, indazolyl, indolyl, isoxazolyl, oxazolyl, perimidinyl, pyridyl, pyrimidinyl, pyrrolyl, quinazolinyl, quinolyl, quinoxalinyl, thiadiazolyl, thianthrenyl, thiazolyl, thienyl, and triazolyl.

As used herein, the descriptor “—CN” represents the cyano group, wherein the carbon and nitrogen atoms are bound together by a triple bond. As used herein, the descriptor “—OH” represents the hydroxy group. The descriptors “C═O” or “C(O)” refers to a carbon atom that is doubly bonded to an oxygen atom.

In general, when a definition for a particular variable includes hydrogen and non-hydrogen (halo, alkyl, aryl, etc.) possibilities, the term “substituent(s) other than hydrogen” refers collectively to the non-hydrogen possibilities for that particular variable.

The term “substituent” refers to a group “substituted” on groups such as an alkyl, haloalkyl, cycloakyl, heterocyclyl, aryl, or heteroaryl group at any atom of that group. In one aspect, the substituent(s) on a group are independently any one single or any combination of two or more of the permissible atoms or groups of atoms delineated for that substituent. In another aspect, a substituent may itself be substituted with any one of the above substituents.

Further, as used herein, the phrase “optionally substituted” means unsubstituted (e.g., substituted with hydrogen (H)) or substituted. As used herein, the term “substituted” means that a hydrogen atom is removed and replaced by a substituent. It is understood that substitution at a given atom is limited by valency.

Descriptors such as “C₆-C₁₀ aryl that is optionally substituted with from 1-4 independently selected R^{c} (and the like) is intended to include both an unsubstituted C₆-C₁₀ aryl group and a C₆-C₁₀ aryl group that is substituted with from 1-4 independently selected R^{c}. The use of a substituent (radical) prefix name such as alkyl without the modifier “optionally substituted” or “substituted” is understood to mean that the particular substituent is unsubstituted. However, the use of “haloalkyl” without the modifier “optionally substituted” or “substituted” is still understood to mean an alkyl group, in which at least one hydrogen atom is replaced by halo.

The details of one or more embodiments of the invention are set forth in the description below. Other features and advantages of the invention will be apparent from the description and from the claims.

### DESCRIPTION OF DRAWINGS

FIG. 1 is a table illustrating the biological activities of the compounds described herein. In vitro and cellular assays were used to evaluate the compounds. γ-secretase protease complex was purified according to the procedure described in Fraering et al, Biochemistry 2004. The effect on APP processing in the presence of a compound described herein was quantified by ELISAs (levels of Aβ40 and Aβ42) and the data is shown in FIG. 1 as a percent inhibition at a particular concentration or by an IC₅₀ value. The effect on Notch processing in the presence of a compound was determined by Western Blot detection of the Notch intracellular domain (NICD) and is reported in FIG. 1 as a percent inhibition at a particular concentration Inhibition of cellular production of human Aβ40 and Aβ42 by the test compound was measured by ELISA assay in which case this data is also illustrated as a percent inhibition at a particular concentration or by an IC₅₀ value. The Chinese Hamster Ovary (CHO) 7 W stable cell line used for these assays expresses wild-type human APP protein. Separately, a human osteosarcoma cell line (U2OS) was used to determine the effect of the compound on Notch processing via a sensitive Notch-Luciferase reporter assay. General cellular toxicity was measured in various wild-type human cell lines with a commercial MTS kit. The compounds delineated in FIG. 1 did not show any significant toxicity in this assay when tested at various concentrations.

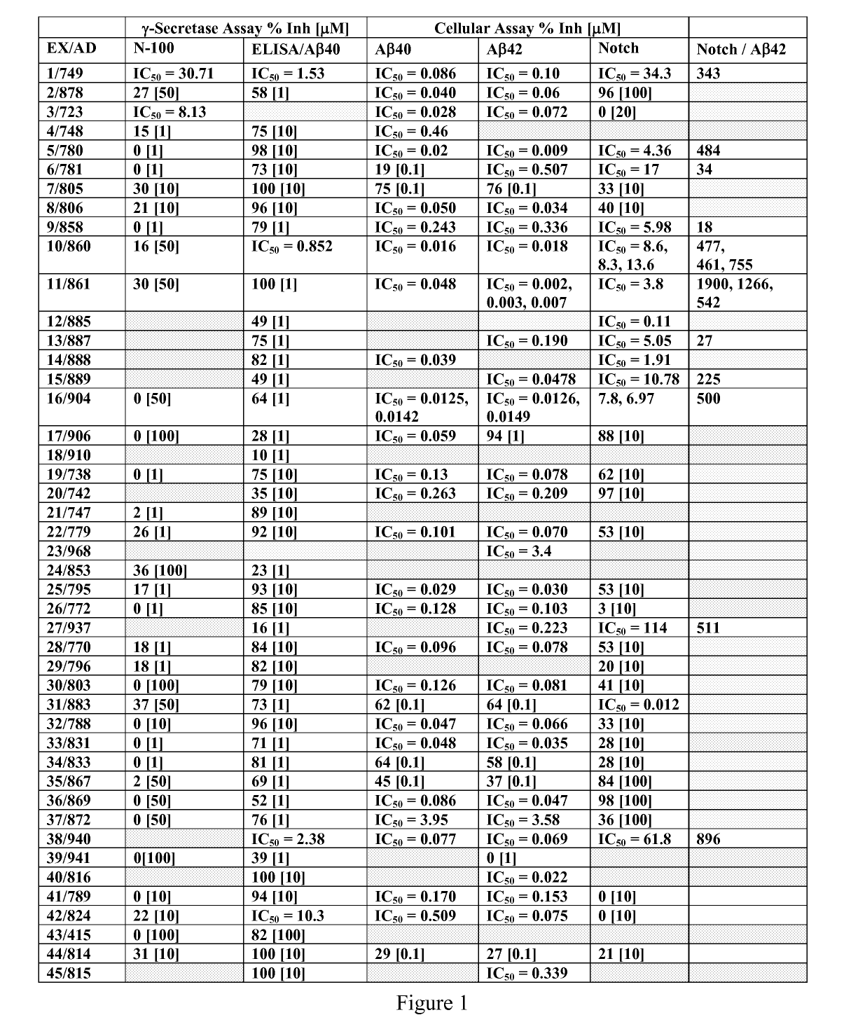

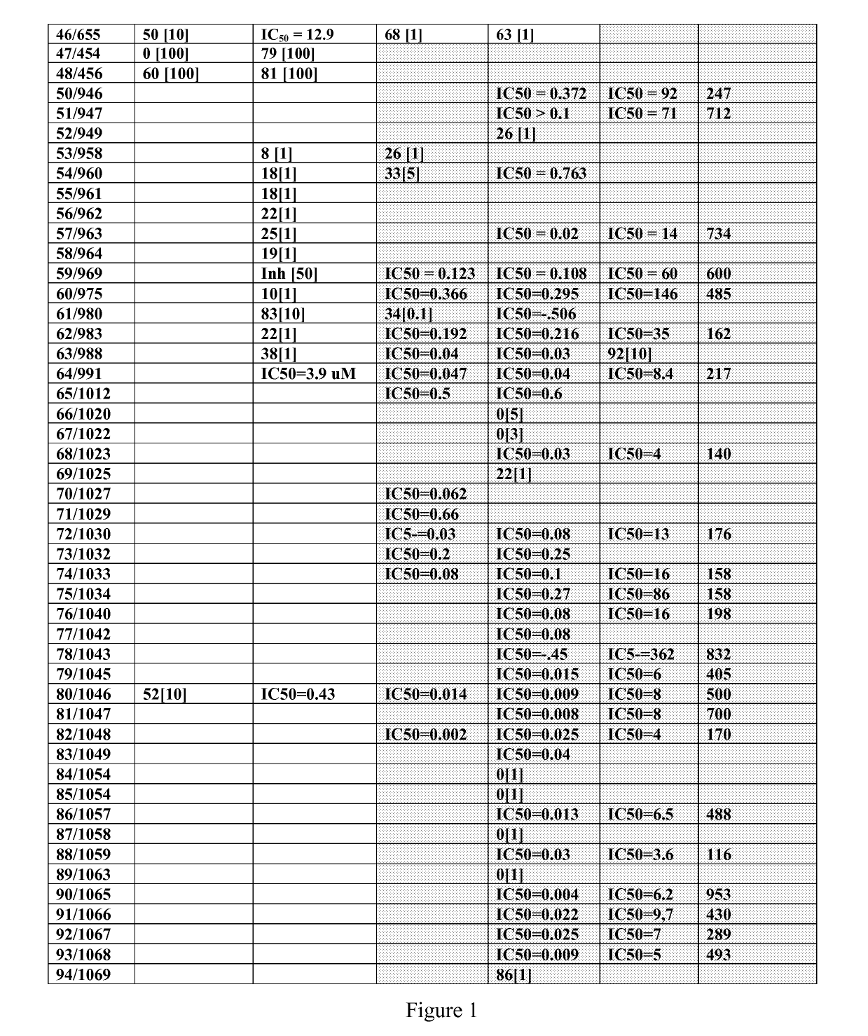

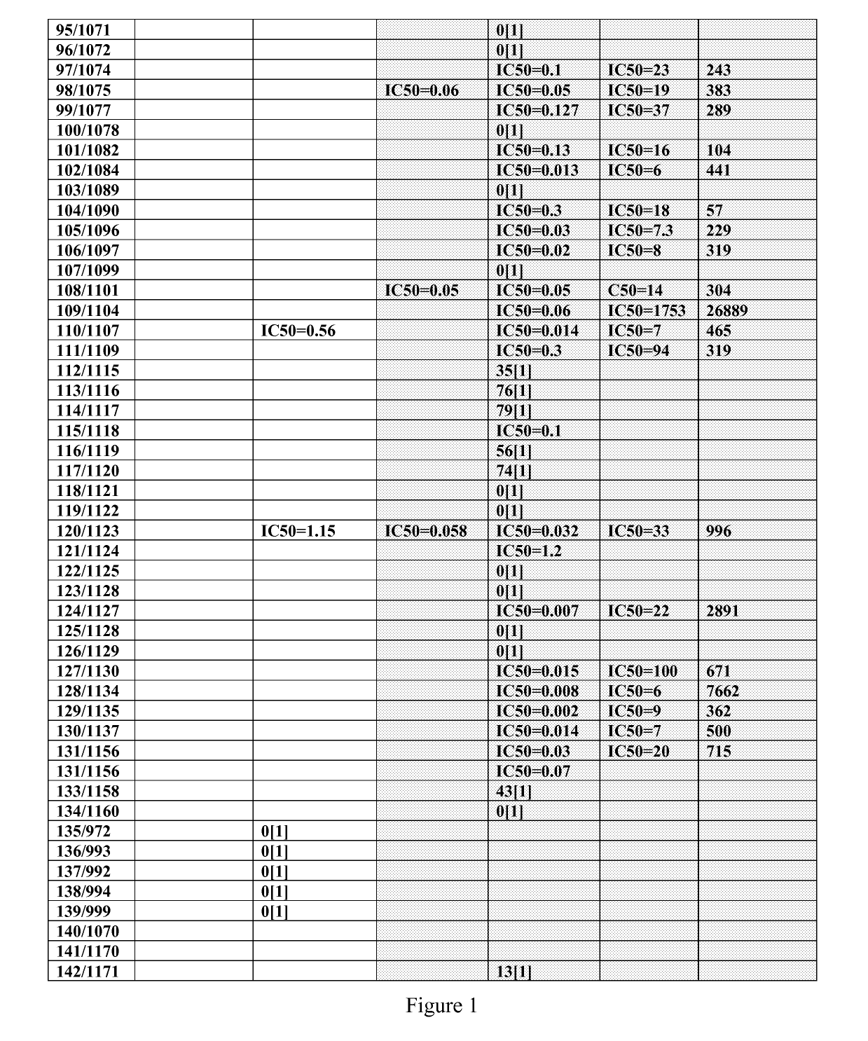

### DETAILED DESCRIPTION

This invention relates generally to the discovery of sulfonamide-containing compounds that are inhibitors of γ-secretase.

In one aspect, compounds having formula (I) are featured:

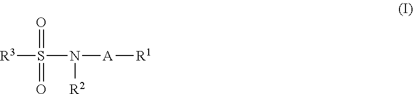

Here and throughout this specification, R¹, R², R³, and A can be as defined anywhere herein.

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable sub-combination.

Thus, for ease of exposition, it is also understood that where in this specification, a variable (e.g., R¹) is defined by “as defined anywhere herein” (or the like), the definitions for that particular variable include the first occurring and broadest generic definition as well as any sub-generic and specific definitions delineated anywhere in this specification.

Variable R¹

As defined above, R¹ has the following formula:

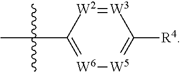

Variables W², W³, W⁵, and W⁶

In some embodiments, each of W², W³, W⁵, and W⁶ is independently selected from CH, C(halo). In some embodiments, the definition of W², W³, W⁵, and W⁶ can further include COR (where R═H, C₁-C₆ alkyl). In these embodiments, R¹ is an optionally substituted phenyl group.

In certain embodiments, each occurrence of C(halo) is CF (in which F represents fluoro).

In certain embodiments, each of W², W³, W⁵, and W⁶ is CH.

In some embodiments, one or two of W², W³, W⁵, and W⁶ are N; and the others are independently selected from CH or C(halo). In certain embodiments, each occurrence of C(halo) is CF.

In certain embodiments, one or two of W², W³, W⁵, and W⁶ are N; and the others are CH.

In certain embodiments, each of W³ and W⁵ is N; and one of W² and W⁶ is CH and the other of W² and W⁶ is C(halo). In certain embodiments, each of W² and W⁶ is CH.

In certain embodiments, one of W² and W³ is N; and the others are independently selected from CH or C (halo). In certain embodiments, one of W² and W³ is N; and the others are CH.

In some embodiments, W², W³, W⁵, and W⁶ are defined according to (A) below:

### A

each of W² and W⁶ is independently selected from CH and C(halo); and
each of W³ and W⁵ is independently selected from CH; C(halo); and CR′; wherein R′ is —C(O)OH, —C(O)O(C₁-C₆ alkyl), or —CN.

In these embodiments, R¹ is an optionally substituted phenyl group.

In certain embodiments, each of W² and W⁶ is CH.

In certain embodiments, each of W³ and W⁵ is other than CR′; i.e., each of W³ and W⁵ is independently selected from CH and C(halo); e.g., each of W³ and W⁵ is CH.

In certain embodiments, each of W², W³, W⁵, and W⁶ is independently selected from CH and C(halo).

In certain embodiments, each of W², W³, W⁵, and W⁶ is CH.

In certain embodiments, one of W³ and W⁵ is CR′, and the other of W³ and W⁵ is CH and C(halo).

Embodiments can include one or more of the following features.

The other of W³ and W⁵ is CH.

Each of W² and W⁶ is CH.

The other of W³ and W⁵ is CH, and each of W² and W⁶ is CH.

R′ is —C(O)OH or —C(O)O(C₁-C₆ alkyl). R′ is —C(O)OH).

In some embodiments, W², W³, W⁵, and W⁶ are defined according to definition (B):
one or two of W², W³, W⁵, and W⁶ are N; and the others are independently selected from CH and C(halo).

In certain embodiments, one or two of W², W³, W⁵, and W⁶ are N; and the others are independently selected from CH or C(halo).

In certain embodiments, one or two of W², W³, W⁵, and W⁶ are N; and the others are CH.

In certain embodiments, one or two of W³ and W⁵ is/are N.

For example, one of W³ and W⁵ is N; the other of W³ and W⁵ is independently selected from CH or C(halo) (e.g., the other of W³ and W⁵ is CH); and each of W² and W⁶ is independently selected from CH and C(halo) (e.g., each of W² and W⁶ is CH).

As another example, each of W³ and W⁵ is N; and one of W² and W⁶ is CH and the other of W² and W⁶ is C(halo). In certain embodiments, each of W² and W⁶ is CH.

In certain embodiments, one of W² and W³ is N; and the others are independently selected from CH or C (halo). In certain embodiments, one of W² and W³ is N; and the others are CH.

In certain of the above described embodiments (for both (A) and (B)), each occurrence of C(halo) is CF (in which F represents fluoro).

Variable R⁴

In some embodiments, R⁴ is selected from any of the substituents delineated in (i)-(v) immediately below:
(i) halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); NHC(O)R⁴¹, —NHSO₂R⁴¹, —SO₂N(R⁴²)(R⁴³); —C(O)NHCH(CH₂OH)₂,
(ii) C₁-C₆ alkoxy, —OCH(CH₂OH)₂, C₁-C₆ thioalkoxy, C₁-C₆ haloalkoxy, C₁-C₆ halothioalkoxy, C₁-C₆ alkyl, C₁-C₆ haloalkyl, each of which is optionally substituted with from 1-3 (e.g., 1-2 or 1) substituents selected from —OH and —CN;
(iii) heterocyclyl, each containing from 3-8 ring atoms, wherein from 1-2 of the ring atoms is independently selected from N, NH, N(C₁-C₆ alkyl), O, and S; and wherein said heterocyclic ring is optionally substituted with from 1-3 independently selected R^{a}; and
(iv) heterocycloalkenyl or heteroaryl, each containing 5 ring atoms, wherein from 1-4 of the ring atoms is independently selected from N, NH, N(C₁-C₆ alkyl), O, and S; and wherein said heteroaryl ring is optionally substituted with from 1-3 independently selected R^{b}.

In some embodiments, R⁴ is selected from any of the substituents delineated in (i)-(v) immediately below:
(i) halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); —NHC(O)R⁴¹, —NHSO₂R⁴¹, —SO₂N(R⁴²)(R⁴³); —C(O)NHCH(CH₂OH)₂, —C(O)NH(CH₂)₃COOH; OCH(CH₂OH)₂;
(ii) C₁-C₆ alkoxy, C₁-C₆ thioalkoxy, C₁-C₆ haloalkoxy, C₁-C₆ halothioalkoxy, C₁-C₆ alkyl, C₁-C₆ haloalkyl, each of which is optionally substituted with from 1-3 (e.g., 1-2 or 1) substituents independently selected from —OH, C₁-C₃ alkoxy, —C(O)OH, —C(O)O(C₁-C₆ alkyl), and —CN;
(iii) heterocyclyl or heterocyclyloxy, each containing from 3-8 ring atoms, wherein from 1-2 of the ring atoms is independently selected from N, NH, N(C₁-C₆ alkyl), O, and S; and wherein said heterocyclyl or heterocyclyloxy is optionally substituted with from 1-3 independently selected R^{a};
(iv) heterocycloalkenyl or heteroaryl, each containing 5 ring atoms, wherein from 1-4 of the ring atoms is independently selected from N, NH, N(C₁-C₆ alkyl), O, and S; and wherein said heteroaryl ring is optionally substituted with from 1-3 independently selected R^{b}.

In certain embodiments, R⁴ is selected from (i), (ii), and (iii) above.

In some embodiments, R⁴ is selected from
(i) halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); NHC(O)R⁴¹, —NHSO₂R⁴¹, —SO₂N(R⁴²)(R⁴³); —C(O)NHCH(CH₂OH)₂, OCH(CH₂OH)₂;
(ii) C₁-C₆ alkyl or C₁-C₆ haloalkyl, each of which is optionally substituted with from 1-3 (e.g., 1-2 or 1) substituents selected from —OH and —CN;
(iii) heterocyclyl, each containing from 3-8 ring atoms, wherein from 1-2 of the ring atoms is independently selected from NH, N(C₁-C₆ alkyl), O, and S; and wherein said heterocyclic ring is optionally substituted with from 1-3 independently selected R^{a}; and
(iv) heterocycloalkenyl or heteroaryl, each containing 5 ring atoms, wherein from 1-4 of the ring atoms is independently selected from N, NH, N(C₁-C₆ alkyl), O, and S; and wherein said heteroaryl ring is optionally substituted with from 1-3 independently selected R^{b}.

In certain embodiments, R⁴ is selected from (i), (iii), and (iv) above.

In some embodiments, R⁴ is selected from:
(i) halo; —CO₂H; —C(O)OR⁴¹; —NHC(O)OR⁴¹; —N(CH₃)C(O)OR⁴¹; C(O)N(R⁴²)(R⁴³); —C(O)R⁴⁴; —CN; —NO₂; —SO₃H; —P(O)(OH)₂; —OH, —SO₂(R⁴⁵); NHC(O)R⁴¹, —NHSO₂R⁴¹, —SO₂N(R⁴²)(R⁴³); —C(O)NHCH(CH₂OH)₂, OCH(CH₂OH)₂;
(ii) C₁-C₆ alkyl or C₁-C₆ haloalkyl, each of which is optionally substituted with from 1-3 (e.g., 1-2 or 1) substituents selected from —OH and —CN; and
(iii) heterocyclyl each containing from 3-8 ring atoms, wherein from 1-2 of the ring atoms is independently selected from NH, N(C₁-C₆ alkyl), O, and S; and wherein said heterocyclic ring is optionally substituted with from 1-3 independently selected R^{a}.

In some embodiments, R⁴ is selected from:
(i) halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); NHC(O)R⁴¹, —NHSO₂R⁴¹, —SO₂N(R⁴²)(R⁴³); —C(O)NHCH(CH₂OH)₂, OCH(CH₂OH)₂; and
(iii) heterocyclyl, each containing from 3-8 ring atoms, wherein from 1-2 of the ring atoms is independently selected from N, NH, N(C₁-C₆ alkyl), O, and S; and wherein said heterocyclic ring is optionally substituted with from 1-3 independently selected R^{a}.

In certain embodiments, R⁴ is selected from any of the substituents delineated in (i)-(iii) immediately below:
(i) halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); —NHC(O)R⁴¹, —NHSO₂R⁴¹, —SO₂N(R⁴²)(R⁴³); —C(O)NHCH(CH₂OH)₂, —C(O)NH(CH₂)₃COOH; OCH(CH₂OH)₂;
(ii) C₁-C₆ alkoxy, C₁-C₆ thioalkoxy, C₁-C₆ haloalkoxy, C₁-C₆ halothioalkoxy, C₁-C₆ alkyl, C₁-C₆ haloalkyl, each of which is optionally substituted with from 1-3 (e.g., 1-2 or 1) substituents independently selected from —OH, C₁-C₃ alkoxy, —C(O)OH, —C(O)O(C₁-C₆ alkyl), and —CN;
(iii) heterocyclyloxy, containing from 3-8 ring atoms, wherein from 1-2 of the ring atoms is independently selected from N, NH, N(C₁-C₆ alkyl), O, and S; and wherein said heterocyclyl or heterocyclyloxy is optionally substituted with from 1-3 independently selected R^{a}.

In embodiments, each of (i), (ii), (iii), and (iv) delineated above can be any subset of substituents as defined anywhere herein.

In some embodiments, R⁴ is selected from COOH, CONHCH₂CH₂OH, CONH—CH₂(CH₂)ₘOH, CONHCH(CH₃)(CH₂)ₘOH,

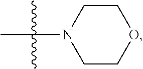 [SMILES: *N1CCOCC1]

NHC(O)R⁴¹, —NHSO₂R⁴¹, —SO₂N(R⁴²)(R⁴³); —C(O)NHCH(CH₂OH)₂, OCH(CH₂OH)₂, NHC(O)OR′ NHC(O)OCH₂CH₃, COOR′, COH(CH₃)₂, SO₂CH₃, SO₂CF₃ COCH₃, whereby m is selected from 1 to 3; R′ is selected from C₁-C₆ alkyl.

In some embodiments, R⁴ is other than hydrogen.

In some embodiments, R⁴ is other than halo.

In some embodiments, R⁴ is other than C₁-C₆ alkoxy, C₁-C₆ thioalkoxy, C₁-C₆ haloalkoxy, C₁-C₆ halothioalkoxy, each of which is optionally substituted with from 1-3 (e.g., 1-2 or 1) substituents selected from —OH and —CN.

In some embodiments, R⁴ is other than hydrogen, halo, C₁-C₆ alkoxy, C₁-C₆ thioalkoxy, C₁-C₆ haloalkoxy, C₁-C₆ halothioalkoxy, each of which is optionally substituted with a substituent selected from —OH and —CN.

In some embodiments, R⁴ is selected from halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, and —SO₂(R⁴⁵), —NHC(O)R⁴¹, —NHSO₂R⁴¹, —SO₂N(R⁴²)(R⁴³); —C(O)NHCH(CH₂OH)₂, OCH(CH₂OH)₂,

In certain embodiments, R⁴ is selected from —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —SO₃H; —P(O)(OH)₂; and —SO₂(R⁴⁵), —NHC(O)R⁴¹, —NHSO₂R⁴¹, —SO₂N(R⁴²)(R⁴³).

In certain embodiments, R⁴ is selected from —CO₂H; —C(O)OR⁴¹; —NHC(O)OR⁴¹; —N(CH₃)C(O)OR⁴¹; C(O)N(R⁴²)(R⁴³); —C(O)R⁴⁴; —CN; and —SO₂(R⁴⁵).

In certain embodiments, R⁴ is selected from halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, C₁-C₆ alkoxy, and —SO₂(R⁴⁵).

In certain embodiments, R⁴ is —CO₂H.

In certain embodiments, R⁴ is —C(O)OR⁴¹. In embodiments, R⁴¹ is C₁-C₈ alkyl (e.g., C₁-C₃ alkyl, e.g., CH₃ or CH₂CH₃; or C₃-C₆ alkyl, e.g., C₃-C₆ branched alkyl, e.g., t-butyl, isopropyl, isobutyl).

In certain embodiments, R⁴ is —SO₂(R⁴⁵). In embodiments, R⁴⁵ is C₁-C₈ alkyl and branched alkyl (e.g., C₁-C₃ alkyl, e.g., CH₃).

In certain embodiments, R⁴ is —C(O)N(R⁴²)(R⁴³).

In embodiments, one of R⁴² and R⁴³ is hydrogen, and the other of R⁴² and R⁴³ is a substituent other than hydrogen.

In embodiments, one of R⁴² and R⁴³ is hydrogen, and the other of R⁴² and R⁴³ is C₁-C₈ alkyl or C₁-C₈ haloalkyl, each of which is optionally substituted with —OH (e.g., C₁-C₈ alkyl, which is optionally substituted with —OH). For example, one of R⁴² and R⁴³ can be hydrogen, and the other of R⁴² and R⁴³ can be C₁-C₈ (e.g., C₁-C₆) alkyl which is substituted with —OH. For example, R⁴ can be CONHCH₂CH₂OH, CONHCH₂(CH₂)ₘOH, or CONHCH(CH₃)(CH₂)ₘOH, in which m is, independently, 1, 2, or 3.

In certain embodiments, each of R⁴² and R⁴³ is independently selected from:
(i) hydrogen;
(ii) C₁-C₈ alkyl; C₁-C₈ haloalkyl; C₃-C₈ cycloalkyl; and heterocyclyl containing from 3-8 ring atoms, wherein from 1-2 of the ring atoms is independently selected from N, NH, N(C₁-C₆ alkyl), O, and S; and wherein each of said alkyl, haloalkyl, cycloalkyl, and heterocyclyl is optionally substituted with from 1-3 (e.g., 1) R^{c}.

In certain embodiments, one of R⁴² and R⁴³ is hydrogen; and the other of R⁴² and R⁴³ is C₁-C₈ alkyl; C₁-C₈ haloalkyl; C₃-C₈ cycloalkyl; and heterocyclyl containing from 3-8 ring atoms, wherein from 1-2 of the ring atoms is independently selected from N, NH, N(C₁-C₆ alkyl), O, and S; and wherein each of said alkyl, haloalkyl, cycloalkyl, and heterocyclyl is optionally substituted with from 1-3 (e.g., 1) R^{c}.

In certain embodiments, one of R⁴² and R⁴³ is hydrogen; and the other of R⁴² and R⁴³ is C₁-C₈ alkyl, which is optionally substituted with from 1-3 (e.g., 1) R^{c}.

In embodiments, R^{c} at each occurrence is, independently, —OH; C₁-C₆ alkoxy (e.g., OCH₃); —C(O)(C₁-C₆ alkyl) (e.g., —C(O)CH₃); or heterocyclyl containing from 5-6 ring atoms, wherein from 1-2 of the ring atoms of the heterocyclyl is independently selected from N, NH, N(C₁-C₆ alkyl), NC(O)(C₁-C₆ alkyl), O, and S; and wherein said heterocyclyl is optionally substituted with from 1-3 substituents independently selected from —OH and C₁-C₄ alkyl (e.g., R^{c} can be pyranyl, e.g., 4-pyranyl).

In embodiments, one of R⁴² and R⁴³ is hydrogen, and the other of R⁴² and R⁴³ is C₁-C₈ alkyl or C₁-C₈ haloalkyl, each of which optionally substituted with —OH (e.g., C₁-C₈ alkyl, which is optionally substituted with —OH). For example, one of R⁴² and R⁴³ is hydrogen, and the other of R⁴² and R⁴³ is C₁-C₈ (e.g., C₁-C₆) alkyl which is substituted with —OH. For example, R⁴ can be CONHCH₂CH₂OH, CONHCH₂(CH₂)ₘOH, or CONHCH(CH₃)(CH₂)ₘOH, in which m is, independently, 1, 2, or 3. In embodiments, when R⁴ is CONHCH(CH₃)(CH₂)ₘOH (e.g., m=1), the carbon attached to CH₃ has the R-configuration.

In certain embodiments, one of R⁴² and R⁴³ is hydrogen; and the other of R⁴² and R⁴³ is C₃-C₈ (e.g., C₃-C₆, e.g., C₅-C₆) cycloalkyl; or heterocyclyl containing from 3-8 (e.g., 3-6, 5-6) ring atoms, wherein from 1-2 of the ring atoms is independently selected from N, NH, N(C₁-C₆ alkyl), O, and S; and wherein each of said cycloalkyl or heterocyclyl is optionally substituted with from 1-3 (e.g., 1) R^{c} (e.g., R^{c} is —OH). For example, the other of R⁴² and R⁴³ can be optionally substituted (e.g., R^{c} is —OH) cyclopentyl or cyclohexyl (e.g., e.g., R^{c} is —OH; e.g., the hydroxylated ring carbon having the R-configuration or the S-configuration); or optionally substituted pyranyl (e.g., 4-pyranyl).

In certain embodiments, R⁴²—N—R⁴³ together forms a saturated ring having 5 or 6 ring atoms, in which from 1 or 2 ring atoms, in addition to the N that occurs between R⁴² and R⁴³, is/are optionally a heteroatom independently selected from NH, N(alkyl), O, or S; and wherein said saturated ring is optionally substituted with from 1-3 R^{c} (e.g., R⁴²—N—R⁴³ together forms a morpholino ring).

In some embodiments, R⁴ is heterocyclyl or heterocyclyloxy, each containing from 3-8 ring atoms, wherein from 1-2 of the ring atoms is independently selected from N, NH, N(C₁-C₆ alkyl), O, and S; and wherein said heterocyclyl or heterocyclyloxy is optionally substituted with from 1-3 independently selected R^{a}.

In certain embodiments, R⁴ is heterocyclyl containing from 3-8 ring atoms, wherein from 1-2 of the ring atoms is independently selected from N, NH, N(C₁-C₆ alkyl), O, and S; and wherein said heterocyclic ring is optionally substituted with from 1-3 independently selected R^{a}. For example, R⁴ can be morpholino (e.g., 4-morpholino, pyrrolidine, piperidine, piperazine).

In certain embodiments, R⁴ is heterocyclyloxy, each containing from 3-8 ring atoms, wherein from 1-2 of the ring atoms is independently selected from N, NH, N(C₁-C₆ alkyl), O, and S; and wherein said heterocyclyloxy is optionally substituted with from 1-3 independently selected R^{a} (e.g., R⁴ can be pyranyloxy, e.g., 4-pyranyloxy; or the hyeterocyclyl portion can be as defined above).

In certain embodiments, R⁴ is selected from —C(O)OR⁴¹; —NHC(O)OR⁴¹; —N(CH₃)C(O)OR⁴¹ (e.g., —C(O)OR⁴¹). In embodiments, each occurrence of R⁴¹ is C₁-C₈ alkyl and branched alkyl (e.g., C₁-C₃ alkyl, e.g., CH₃ or CH₂CH₃; or C₃-C₆ alkyl, e.g., C₃-C₆ branched alkyl, e.g., t-butyl, isopropyl, isobutyl).

In certain embodiments, R⁴ is —C(O)R⁴⁴. In certain embodiments, R⁴⁴ is C₁-C₈ alkyl and branched alkyl (e.g., C₁-C₃ alkyl, e.g., CH₃).

In some embodiments, R⁴ is heterocyclyl, each containing from 3-8 ring atoms, wherein from 1-2 of the ring atoms is independently selected from N, NH, N(C₁-C₆ alkyl), O, and S; and wherein said heterocyclic ring is optionally substituted with from 1-3 independently selected R^{a}.

In certain embodiments, R⁴ is heterocyclyl containing from 3-8 ring atoms, wherein from 1-2 of the ring atoms is independently selected from N, NH, N(C₁-C₆ alkyl), O, and S; and wherein said heterocyclic ring is optionally substituted with from 1-3 independently selected R^{a}. For example, R⁴ can be morpholino (e.g., 4-morpholino, pyrrolidine, piperidine, piperazine).

In some embodiments, R⁴ is heterocycloalkenyl or heteroaryl, each containing 5 ring atoms, wherein from 1-4 of the ring atoms is independently selected from N, NH, N(C₁-C₆ alkyl), O, and S; and wherein said heteroaryl ring is optionally substituted with from 1-3 independently selected R^{b}.

In some embodiments, R⁴ is C₁-C₆ alkyl or C₁-C₆ haloalkyl, each of which is optionally substituted with a substituent selected from —OH and —CN.

In certain embodiments, R⁴ is C₁-C₈ alkyl (e.g., C₁-C₃ alkyl, e.g., CH₃ or CH₂CH₃; or C₃-C₆ alkyl, e.g., C₃-C₆ branched alkyl, e.g., t-butyl, isopropyl, isobutyl) that is optionally substituted with —OH.

In some embodiments, R⁴ is C₁-C₆ alkoxy, C₁-C₆ thioalkoxy, C₁-C₆ haloalkoxy, C₁-C₆ halothioalkoxy, C₁-C₆ alkyl, or C₁-C₆ haloalkyl, each of which is optionally substituted with from 1-3 (e.g., 1-2 or 1) substituents independently selected from —OH, C₁-C₃ alkoxy, —C(O)OH, —C(O)O(C₁-C₆ alkyl), and —CN.

In certain embodiments, R⁴ is C₁-C₆ alkoxy, C₁-C₆ thioalkoxy, C₁-C₆ haloalkoxy, or C₁-C₆ halothioalkoxy, each of which is optionally substituted with from 1-3 (e.g., 1-2 or 1) substituents independently selected from —OH, C₁-C₃ alkoxy, —C(O)OH, —C(O)O(C₁-C₆ alkyl), and —CN.

In certain embodiments, R⁴ is C₁-C₆ alkoxy or C₁-C₆ haloalkoxy (e.g., C₁-C₆ alkoxy), each of which is optionally substituted with from 1-3 (e.g., 1-2 or 1) substituents independently selected from —OH, C₁-C₃ alkoxy, —C(O)OH, —C(O)O(C₁-C₆ alkyl), and —CN. For example, R⁴ can be —OCH₃.

In certain embodiments, R⁴ is C₁-C₆ thioalkoxy or C₁-C₆ halothioalkoxy (e.g., C₁-C₆ thioalkoxy), each of which is optionally substituted with from 1-3 (e.g., 1-2 or 1) substituents independently selected from —OH, C₁-C₃ alkoxy, —C(O)OH, —C(O)O(C₁-C₆ alkyl), and —CN. For example, R⁴ can be —SCH₃.

Non-Limiting Combinations of Variables W², W³, W⁵, and W⁶, and R⁴

In some embodiments:

each of W², W³, W⁵, and W⁶ is independently selected from CH or C(halo) or N; and

R⁴ is selected from:
(i) halo; —CO₂H; CH₂OH, —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); NHC(O)R⁴¹, —NHSO₂R⁴¹, —SO₂N(R⁴²)(R⁴³); —C(O)NHCH(CH₂OH)₂, OCH(CH₂OH)₂, - and
(iii) heterocyclyl each containing from 3-8 ring atoms, wherein from 1-2 of the ring atoms is independently selected from N, NH, N(C₁-C₆ alkyl), O, and S; and wherein said heterocyclic ring is optionally substituted with from 1-3 independently selected R^{a}.

In embodiments, W², W³, W⁵, and W⁶, and R⁴ can be further defined as described anywhere herein. For example, embodiments can include one or more of the features delineated below (e.g., embodiments can include a feature below that further defines W², W³, W⁵, and W⁶; and/or one or more features that further define R⁴):
each of W², W³, W⁵, and W⁶ is CH;
R⁴ is selected from —CO₂H; CH₂OH, —C(O)OR⁴¹; —NHC(O)OR⁴¹; —N(CH₃)C(O)OR⁴¹; —C(O)N(R⁴²)(R⁴³); —C(O)R⁴⁴; —CN; —SO₂(R⁴⁵) and —NHC(O)R⁴¹, —NHSO₂R⁴¹, —SO₂N(R⁴²)(R⁴³); —C(O)NHCH(CH₂OH)₂, OCH(CH₂OH)₂ in which R⁴¹, R⁴², R⁴³, R⁴⁴, and R⁴⁵ can be as defined anywhere herein.
R⁴ is —CO₂H;
R⁴ is —SO₂(R⁴⁵), in which R⁴⁵ can be as defined anywhere herein;
R⁴ is —C(O)N(R⁴²)(R⁴³), in which R⁴² and R⁴³, can each be independently as defined anywhere herein;
R⁴ is heterocyclyl, each containing from 3-8 (e.g., 3-6 or 5-7) ring atoms, wherein from 1-2 of the ring atoms is independently selected from N, NH, N(C₁-C₆ alkyl), O, and S; and wherein said heterocyclic ring is optionally substituted with from 1-3 independently selected R^{a};
R⁴ can further include the substituents C₁-C₆ alkyl or C₁-C₆ haloalkyl, each of which is optionally substituted with from 1-3 (e.g., 1-2 or 1) substituents selected from —OH and —CN.

In certain embodiments, each of W², W³, W⁵, and W⁶ is CH; and R⁴ is —CO₂H.

In certain embodiments, each of W², W³, W⁵, and W⁶ is CH; and R⁴ is —SO₂(R⁴⁵), in which R⁴⁵ can be as defined anywhere herein.

In some of the above-described R⁴ embodiments, W², W³, W⁵, and W⁶ are defined according to definition (A) as defined anywhere herein. Non-limiting examples of W², W³, W⁵, and W⁶ include:
each of W², W³, W⁵, and W⁶ is CH; and
one of W³ and W⁵ is CR′, and the other of W³ and W⁵ is CH, and each of W² and W⁶ is CH.

In certain embodiments, each of W², W³, W⁵, and W⁶ is CH; and R⁴ is —CO₂H; —C(O)OR⁴¹; —C(O)N(R⁴²)(R⁴³); —SO₂(R⁴⁵), or heterocyclyloxy.

In certain embodiments, each of W², W³, W⁵, and W⁶ is CH; and R⁴ is —CO₂H; —C(O)OR⁴¹; —C(O)N(R⁴²)(R⁴³); or —SO₂(R⁴⁵).

In certain embodiments, each of W², W³, W⁵, and W⁶ is CH; and R⁴ is —CO₂H.

In certain embodiments, one of W³ and W⁵ is CR′ (e.g., CCO₂H) and the other of W³ and W⁵ is CH, and each of W² and W⁶ is CH, and R⁴ can be, e.g., H or C₁-C₆ alkoxy (e.g., OCH₃).

In some of the above-described R⁴ embodiments, W², W³, W⁵, and W⁶ are defined according to definition (B) as defined anywhere herein.

In some embodiments, one or more of the following (a) through (h) can apply:

(a) R⁴ is other than hydrogen.

(b) R⁴ is other than halo.

(c) R⁴ is other than C₁-C₆ alkoxy, C₁-C₆ thioalkoxy, C₁-C₆ haloalkoxy, or C₁-C₆ halothioalkoxy, each of which is optionally substituted with from 1-3 (e.g., 1-2 or 1) substituents independently selected from —OH, C₁-C₃ alkoxy, —C(O)OH, —C(O)O(C₁-C₆ alkyl), and —CN.

(d) R⁴ is other than hydrogen, halo, C₁-C₆ alkoxy, C₁-C₆ thioalkoxy, C₁-C₆ haloalkoxy, or C₁-C₆ halothioalkoxy, each of which is optionally substituted with from 1-3 (e.g., 1-2 or 1) substituents independently selected from —OH, C₁-C₃ alkoxy, —C(O)OH, —C(O)O(C₁-C₆ alkyl), and —CN.

(e) R⁴ is C₁-C₆ alkoxy or C₁-C₆ haloalkoxy (e.g., C₁-C₆ alkoxy), each of which is optionally substituted with from 1-3 (e.g., 1-2 or 1) substituents independently selected from —OH, C₁-C₃ alkoxy, —C(O)OH, —C(O)O(C₁-C₆ alkyl), and —CN; and

W², W³, W⁵, and W⁶ are defined according to definition (A);

and

one of W³ and W⁵ is CR′ (e.g., R′ is —C(O)OH or —C(O)O(C₁-C₆ alkyl); e.g., —C(O)OH).

(f) In certain embodiments, it is provided that when R⁴ is C₁-C₆ alkoxy or C₁-C₆ haloalkoxy (e.g., C₁-C₆ alkoxy), each of which is optionally substituted with from 1-3 (e.g., 1-2 or 1) substituents independently selected from —OH, C₁-C₃ alkoxy, —C(O)OH, —C(O)O(C₁-C₆ alkyl), and —CN; then W², W³, W⁵, and W⁶ are defined according to definition (A); and one of W³ and W⁵ is CR′ (e.g., —C(O)OH or —C(O)O(C₁-C₆ alkyl); e.g., —C(O)OH).

(g) R⁴ is C₁-C₆ alkoxy or C₁-C₆ haloalkoxy (e.g., C₁-C₆ alkoxy), each of which is optionally substituted with from 1-3 (e.g., 1-2 or 1) substituents independently selected from —OH, C₁-C₃ alkoxy, —C(O)OH, —C(O)O(C₁-C₆ alkyl), and —CN; and

W², W³, W⁵, and W⁶ are defined according to definition (A);

and

one or more of (or two or more of) W², W³, W⁵, and W⁶ is independently selected from C(halo (e.g., CF).

(h) In certain embodiments, it is provided that when R⁴ is C₁-C₆ alkoxy or C₁-C₆ haloalkoxy (e.g., C₁-C₆ alkoxy), each of which is optionally substituted with from 1-3 (e.g., 1-2 or 1) substituents independently selected from —OH, C₁-C₃ alkoxy, —C(O)OH, —C(O)O(C₁-C₆ alkyl), and —CN; and W², W³, W⁵, and W⁶ are defined according to definition (A); and one or more of (or two or more of) W², W³, W⁵, and W⁶ is independently selected from C(halo (e.g., CF).

Variable A

In some embodiments, A is CH₂ (i.e., each of R^{A} is hydrogen).

Variable R²

As defined above R² has the following formula:

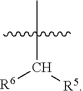

Variable R⁵

In some embodiments, R⁵ is:
(i) C₆-C₁₀ aryl, which is optionally substituted with from 1-3 independently selected R^{c}; or
(ii) heteroaryl containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{c}.

In some embodiments, R⁵ is C₆-C₁₀ aryl, which is optionally substituted with from 1-3 independently selected R^{c}.

In certain embodiments, R^{c} at each occurrence is independently selected from the substituents listed in (aa) and (bb) in the definition of R^{c}. In certain embodiments, R^{c} at each occurrence is independently selected from the listed in (aa) in the definition of R^{c}. In certain embodiments, R^{c} at each occurrence is independently selected from the listed in (bb) in the definition of R^{c}.

In certain embodiments, R^{c} at each occurrence is independently selected from halo; C₁-C₆ alkoxy; C₁-C₆ haloalkoxy; C₁-C₆ thioalkoxy; C₁-C₆ thiohaloalkoxy; C₁-C₆ alkyl and branched alkyl, C₁-C₆ haloalkyl; —CN; —C(O)(C₁-C₆ alkyl); C(O)OH; —C(O)O(C₁-C₆ alkyl); —SO₂(C₁-C₆ alkyl), and —SO₂(C₁-C₆ haloalkyl), —C(O)NR′″R″″—SO₂NR′″R″″, —SO₂NH₂, —NHCO(C₁-C₆ alkyl), —NHSO₂(C₁-C₆ alkyl), whereby R′″ and R″″ is independently selected from H, C₁-C₆ alkyl, C₁-C₆ haloalkyl.

In certain embodiments, R^{c} at each occurrence is independently selected from halogen (e.g., fluoro or chloro), CH₃, OCH₃, CN, OCF₃, COCH₃, COOH, SO₂CH₃, SO₂CF₃, COCH₃, COOCH₃, SO₂NH₂, CF₃.

In certain embodiments, R^{c} at each occurrence is independently selected from halo; C₁-C₆ alkoxy; C₁-C₆ haloalkoxy; C₁-C₆ thioalkoxy; C₁-C₆ thiohaloalkoxy; C₁-C₆ alkyl and branched alkyl, and C₁-C₆ haloalkyl.

In certain embodiments, R^{c} at each occurrence is independently selected from halo; C₁-C₆ alkoxy; C₁-C₆ haloalkoxy; C₁-C₆ alkyl; and C₁-C₆ haloalkyl. For example, R^{c} at each occurrence is independently selected from halogen (e.g., fluoro or chloro), CH₃, OCH₃, OCF₃, and, CF₃.

In certain embodiments, R^{c} at each occurrence is independently selected from halo (e.g., fluoro or chloro).

In certain embodiments, R^{c} at each occurrence is independently selected from —CN; —C(O)(C₁-C₆ alkyl); C(O)OH; —C(O)O(C₁-C₆ alkyl); —SO₂(C₁-C₆ alkyl), —C(O)NR′″R″″—SO₂NR′″R″″, —SO₂NH₂, —NHCO(C₁-C₆ alkyl), —NHSO₂ (C₁-C₆ alkyl), whereby R′″ and R″″ is independently selected from H, C₁-C₆ alkyl, C₁-C₆ haloalkyl.

For example, R^{c} at each occurrence is independently selected from CN, COCH₃, COOH, SO₂CH₃, SO₂CF₃, COCH₃, and COOCH₃.

In certain embodiments, R^{c} is not selected from the substituents listed in (bb), e.g., R^{c} is not —SO₂(C₁-C₆ alkyl).

In some embodiments, R⁵ is phenyl, which is optionally substituted with from 1-3 independently selected R^{c}.

In certain embodiments, R⁵ is unsubstituted phenyl.

In certain embodiments, R⁵ is phenyl that is substituted with 1 or 2 (e.g., 1) R^{c}, in which R^{c} can be as defined anywhere herein. In embodiments, R^{c} or at least one R^{c} is attached to the phenyl ring carbon that is para with respect to the phenyl ring carbon that is attached to the central carbon atom of R².

In some embodiments, R⁵ is heteroaryl containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{c}, in which R^{c} can be as defined anywhere herein.

In certain embodiments, R⁵ is heteroaryl containing from 5-6 ring atoms, wherein from 1-4 of the ring atoms is independently selected from N, NH, N(C₁-C₆ alkyl), O, and S; and wherein said heteroaryl ring is optionally substituted with from 1-3 independently selected R^{c}, in which R^{c} can be as defined anywhere herein. For example, R⁵ can be optionally substituted pyridyl.

In some embodiments, R⁵ is C₁-C₆ alkyl, which is optionally substituted with a substituent selected from —OH and —CN (e.g., —OH).

In certain embodiments, R⁵ is C₁-C₄ alkyl, which is optionally substituted with a substituent selected from —OH and —CN (e.g., —OH).

In certain embodiments, R⁵ is —CH₃, which is optionally substituted with a substituent selected from —OH and —CN (e.g., —OH). In embodiments, R⁵ is —CH₃.

In certain embodiments, R⁵ is —CH₂CH₃, which is optionally substituted with a substituent selected from —OH and —CN (e.g., —OH). In embodiments, R⁵ is —CH₂CH₃.

In some embodiments, R⁵ is C₁-C₆ haloalkyl, each of which is optionally substituted with a substituent selected from —OH and —CN.

In certain embodiments, R⁵ is C₁-C₄ haloalkyl, which is optionally substituted with a substituent selected from —OH and —CN (e.g., —OH).

In certain embodiments, R⁵ is C₁-C₃ haloalkyl (e.g., CF₃).

Variable R⁶

In some embodiments, R⁶ is C₁-C₆ alkyl, which is optionally substituted with a substituent selected from —OH and —CN (e.g., —OH).

In certain embodiments, R⁶ is C₁-C₄ alkyl, which is optionally substituted with a substituent selected from —OH and —CN (e.g., —OH).

In certain embodiments, R⁶ is —CH₃, which is optionally substituted with a substituent selected from —OH and —CN (e.g., —OH). In embodiments, R⁶ is —CH₃.

In certain embodiments, R⁶ is —CH₂CH₃, which is optionally substituted with a substituent selected from —OH and —CN (e.g., —OH). In embodiments, R⁶ is —CH₂CH₃.

In some embodiments, R⁶ is C₁-C₆ haloalkyl, each of which is optionally substituted with a substituent selected from —OH and —CN.

In certain embodiments, R⁶ is C₁-C₄ haloalkyl, which is optionally substituted with a substituent selected from —OH and —CN (e.g., —OH).

In certain embodiments, R⁶ is C₁-C₃ haloalkyl (e.g., CF₃).

Non-Limiting Combinations of R⁵ and R⁶

In some embodiments:

R⁵ is C₆-C₁₀ aryl, which is optionally substituted with from 1-3 independently selected R^{c}; and

R⁶ is C₁-C₆ alkyl, which is optionally substituted with a substituent selected from —OH, F and —CN (e.g., —OH).

In embodiments, R⁵ and R⁶ can be further defined as described anywhere herein. For example, embodiments can include one or more of the features delineated below (e.g., embodiments can include one or more features below that further defines R⁵ and/or one or more features that further define R⁶):
R⁵ is unsubstituted phenyl;
R⁵ is phenyl that is substituted with 1 or 2 (e.g., 1) R^{c}, in which R^{c} can be as defined anywhere herein; R^{c} at each occurrence is independently selected from halo; C₁-C₆ alkoxy; C₁-C₆ haloalkoxy; C₁-C₆ thioalkoxy; C₁-C₆ thiohaloalkoxy; C₁-C₆ alkyl and branched alkyl, C₁-C₆ haloalkyl; —CN; —C(O)(C₁-C₆ alkyl); C(O)OH; —C(O)O(C₁-C₆ alkyl); and —SO₂(C₁-C₆ alkyl); C(O)NR′″R″″—SO₂NR′″R″″, —SO₂NH₂, NHCO(C₁-C₆ alkyl), NHSO₂(C₁-C₆ alkyl), whereby R′″ and R″″ is independently selected from H, C₁-C₆ alkyl, C₁-C₆ haloalkyl.
R^{c} at each occurrence is independently selected from halo (e.g., fluoro or chloro).
R⁶ is C₁-C₄ alkyl, which is optionally substituted with a substituent selected from —OH and —CN (e.g., —OH);
R⁶ is —CH₃;
R⁶ is —CH₂CH₃.
When the carbon attached to R⁵ and R⁶ is substituted with four different substituents, the carbon attached to R⁵ and R⁶ can have the R configuration.
When the carbon attached to R⁵ and R⁶ is substituted with four different substituents, the carbon attached to R⁵ and R⁶ can have the S configuration.

In certain embodiments, R⁵ is unsubstituted phenyl, and R⁶ is —CH₂CH₃.

In some embodiments:

R⁵ is C₁-C₆ alkyl, which is optionally substituted with a substituent selected from —OH and —CN (e.g., —OH); and

R⁶ is C₁-C₆ alkyl, which is optionally substituted with a substituent selected from —OH and —CN (e.g., —OH).

In certain embodiments, each of R⁵ and R⁶ is, independently, —CH₃ or —CH₂CH₃, each optionally substituted with a substituent selected from —OH and —CN (e.g., —OH).

Variable R³

In some embodiments, R³ is C₆-C₁₀ aryl, which is optionally substituted with from 1-3 independently selected R^{d}.

In embodiments, R^{d} at each occurrence is independently selected from halo (e.g., fluoro or chloro).

In certain embodiments, R³ is C₆-C₁₀ aryl, which is substituted with from 1-3 independently selected R^{d}, in which R^{d} can be as defined anywhere herein.

In certain embodiments, R³ is phenyl, which is substituted with from 1-3 independently selected R^{d}, in which R^{d} can be as defined anywhere herein. In certain embodiments, R³ is phenyl that is substituted with 1 or 2 (e.g., 1) R^{d}, in which R^{d} can be as defined anywhere herein. In certain embodiments, R^{d} or at least one R^{d} is attached to the phenyl ring carbon that is para with respect to the phenyl ring carbon that is attached to the sulfur atom of the sulfonyl group. For example, R³ can be 4-chloro-phenyl, 4-fluoro-phenyl, or 2,4-difluorophenyl. In certain embodiments, R^{d} or at least one R^{d} is attached to the phenyl ring carbon that is meta with respect to the phenyl ring carbon that is attached to the sulfur atom of the sulfonyl group.

In some embodiments, R³ is heteroaryl containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{d}, in which R^{d} can be as defined anywhere herein.

In certain embodiments, R³ is heteroaryl containing from 5-6 ring atoms, wherein from 1-4 of the ring atoms is independently selected from N, NH, N(C₁-C₆ alkyl), O, and S; and wherein said heteroaryl ring is optionally substituted with from 1-3 independently selected R^{d}, in which R^{d} can be as defined anywhere herein.

In certain embodiments, R³ is heteroaryl containing from 5-6 ring atoms, wherein from 1-4 of the ring atoms is independently selected from N, NH, N(C₁-C₆ alkyl), O, and S; and wherein said heteroaryl ring is substituted with from 1-3 (e.g., 1 or 2, e.g., 1) independently selected R^{d}, in which R^{d} can be as defined anywhere herein. For example, R³ can be optionally substituted thienyl, e.g., 5-chlorothienyl.

Non-Limiting Combinations of R¹, A, R² and R³

[I-A]

In some embodiments:
each of W², W³, W⁵, and W⁶ is independently selected from CH or C(halo),
N;
R⁴ is selected from:
   (i) halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); NHC(O)R⁴¹, —NHSO₂R⁴¹, —SO₂N(R⁴²)(R⁴³), —C(O)NHCH(CH₂OH)₂, OCH(CH₂OH)₂;
   (ii) C₁-C₆ alkyl, and branched alkyl or C₁-C₆ haloalkyl, each of which is optionally substituted with from 1-3 (e.g., 1-2 or 1) substituents selected from —OH and —CN; and
   (iii) heterocyclyl, each containing from 3-8 ring atoms, wherein from 1-2 of the ring atoms is independently selected from N, NH, N(C₁-C₆ alkyl), O, and S; and wherein said heterocyclic ring is optionally substituted with from 1-3 independently selected R^{a};
or
R⁴ is selected from:
   (i) halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); and NHC(O)R⁴¹, —NHSO₂R⁴¹, —SO₂N(R⁴²)(R⁴³), —C(O)NHCH(CH₂OH)₂, OCH(CH₂OH)₂;
   (iii) heterocyclyl containing from 3-8 ring atoms, wherein from 1-2 of the ring atoms is independently selected from NH, N(C₁-C₆ alkyl), O, and S; and wherein said heterocyclic ring is optionally substituted with from 1-3 independently selected R^{a};
A is CH₂;
R⁵ and R⁶ are defined according to (C); and R⁵ is:
   (ii) C₆-C₁₀ aryl, which is optionally substituted with from 1-3 independently selected R^{c}; or
   (iii) heteroaryl containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{c}; and
R³ is C₆-C₁₀ aryl, which is optionally substituted with from 1-3 independently selected R^{d}.

[I-B]

In some embodiments, W², W³, W⁵, W⁶, R⁴, A, R⁵, and R⁶ can be as defined in [I-A], and R³ is heteroaryl containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{d}.

[I-C]

In some embodiments, W², W³, W⁵, W⁶, R⁴, A, and R³ can be as defined in [I-A] or [I-B], and R⁵ is C₆-C₁₀ aryl, which is optionally substituted with from 1-3 independently selected R^{c}.

[I-D]

In some embodiments, W², W³, W⁵, W⁶, R⁴, A, and R³ can be as defined in [I-A] or [I-B], and R⁵ is heteroaryl containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{c}.

[I-E]

In some embodiments, R⁴, A, R⁵, R⁶, and R³ can be as defined in [I-A], [I-B], [I-C], or [I-D], and one or two of W², W³, W⁵, and W⁶ are N; and the others are independently selected from CH or C(halo).

[I-F]

In some embodiments, W², W³, W⁵, W⁶, R⁴, A, and R³ can be as defined in [I-A], [I-B], [I-C], [I-D], or [I-E], and R⁵ is C₁-C₆ alkyl or C₁-C₆ haloalkyl, each of which is optionally substituted with a substituent selected from —OH and —CN (e.g., C₁-C₆ alkyl, which is optionally substituted with a substituent selected from —OH and —CN).

[I-G]

In some embodiments:
each of W², W³, W⁵, and W⁶ is independently selected from CH or C(halo) or N;
R⁴ is selected from:
   (i) halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); NHC(O)R⁴¹, —NHSO₂R⁴¹, —SO₂N(R⁴²)(R⁴³), —C(O)NHCH(CH₂OH)₂, OCH(CH₂OH)₂;
   and
   (iii) heterocyclyl each containing from 3-8 ring atoms, wherein from 1-2 of the ring atoms is independently selected from NH, N(C₁-C₆ alkyl), O, and S; and wherein said heterocyclic ring is optionally substituted with from 1-3 independently selected R^{a};
A is CH₂;
R⁵ is C₆-C₁₀ aryl, which is optionally substituted with from 1-3 independently selected R^{c};
R⁶ is C₁-C₆ alkyl, which is optionally substituted with a substituent selected from —OH and —CN (e.g., —OH); and
R³ can be as defined anywhere herein, e.g., R³ is C₆-C₁₀ aryl, which is substituted with from 1-3 independently selected R^{d}, in which R^{d} can be as defined anywhere herein; or R³ is heteroaryl containing from 5-6 ring atoms, wherein from 1-4 of the ring atoms is independently selected from N, NH, N(C₁-C₆ alkyl), O, and S; and wherein said heteroaryl ring is substituted with from 1-3 (e.g., 1 or 2, e.g., 1) independently selected R^{d}, in which R^{d} can be as defined anywhere herein.

[I-H]

In some embodiments:
each of W², W³, W⁵, and W⁶ is CH;
R⁴ is —CO₂H or —SO₂(R⁴⁵), in which R⁴⁵ can be as defined anywhere herein;
A is CH₂;
R⁵ is unsubstituted phenyl or phenyl substituted with 1 R^{c} (e.g., unsubstituted phenyl),
R⁶ is —CH₃ or —CH₂CH₃; and
R³ can be as defined anywhere herein, e.g., R³ is phenyl that is substituted with 1 or 2 (e.g., 1) R^{d}, or R³ is thienyl that is substituted with 1 or 2 (e.g., 1) R^{d}.

[I-I]

In some embodiments, the compounds can have the following formula

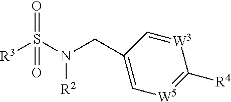

in which:

R³ is selected from 4-chloro-phenyl, 4-fluoro-phenyl, 5-chloro-thiophenyl, 2,4-difluorophenyl, or phenyl substituted with halogen (F, Cl, Br, I);

each of W³ and W⁵ is CH or N; or W³ is N and W⁵ is CH;

R⁴ is selected from COOH, CONH—CH₂—CH₂—OH, C(O)NH—CH₂—(CH₂)ₘ—OH, C(O)NH—CH(CH₃)—(CH₂)ₘ—OH,

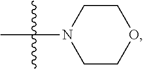 [SMILES: *N1CCOCC1]

NHCOOR′ NHCOOCH₂CH₃, COOR′, C(OH)(CH₃)₂, SO₂CH₃, SO₂CF₃, COCH₃, NHC(O)R⁴¹, —NHSO₂R⁴¹, —SO₂N(R⁴²)(R⁴³); —C(O)NHCH(CH₂OH)₂, OCH(CH₂OH)₂, whereby m is selected from 1 to 3; R′ is selected from C₁-C₆ alkyl;

R² is R′CH₂—CH—R″, in which the bolded carbon (C) is the carbon attached to the sulfonamide nitrogen in formula (I); R′ is H, CH₃, OH, CH₂OH, F, CN; and R″ is selected from methyl, ethyl, phenyl and substituted phenyl, hetero aromatic ring, and substituted hetero aromatic ring, CH₂OH, whereby substitution group is selected from H, halogen (F, Cl), CH₃, OCH₃, CN, OCF₃, C(O)CH₃, COOH, SO₂CH₃, SO₂CF₃, COOCH₃, CF₃.

[I-J]

In some embodiments:
W², W³, W⁵, and W⁶ are defined according to definition (A) as defined anywhere herein; and
R⁴ is selected from any of the substituents delineated in (i)-(iii) immediately below:
   (i) halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); —NHC(O)R⁴¹, —NHSO₂R⁴¹, —SO₂N(R⁴²)(R⁴³); —C(O)NHCH(CH₂OH)₂, —C(O)NH(CH₂)₃COOH; OCH(CH₂OH)₂;
   (ii) C₁-C₆ alkoxy, C₁-C₆ thioalkoxy, C₁-C₆ halo alkoxy, C₁-C₆ halothioalkoxy, C₁-C₆ alkyl, C₁-C₆ haloalkyl, each of which is optionally substituted with from 1-3 (e.g., 1-2 or 1) substituents independently selected from —OH, C₁-C₃ alkoxy, —C(O)OH, —C(O)O(C₁-C₆ alkyl), and —CN;
   (iii) heterocyclyloxy, each containing from 3-8 ring atoms, wherein from 1-2 of the ring atoms is independently selected from N, NH, N(C₁-C₆ alkyl), O, and S; and wherein said heterocyclyl or heterocyclyloxy is optionally substituted with from 1-3 independently selected R^{a};
or
R⁴ is selected from:
   (i) halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); —NHC(O)R⁴¹, —NHSO₂R⁴¹, —SO₂N(R⁴²)(R⁴³); —C(O)NHCH(CH₂OH)₂, —C(O)NH(CH₂)₃COOH; OCH(CH₂OH)₂;
   (iii) heterocyclyloxy, each containing from 3-8 ring atoms, wherein from 1-2 of the ring atoms is independently selected from N, NH, N(C₁-C₆ alkyl), O, and S; and wherein said heterocyclyl or heterocyclyloxy is optionally substituted with from 1-3 independently selected R^{a}; and
A is CH₂; and
R⁵ and R⁶ are defined according to (C); R⁵ is:
   (ii) C₆-C₁₀ aryl, which is optionally substituted with from 1-3 independently selected R^{c}; or
   (iii) heteroaryl containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{c}; and;
R³ is C₆-C₁₀ aryl, which is optionally substituted with from 1-3 independently selected R^{d}.

[I-K]

In some embodiments, W², W³, W⁵, W⁶, R⁴, A, R⁵, and R⁶ can be as defined in [I-J], and R³ is heteroaryl containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{d}.

[I-L]

In some embodiments, each of W², W³, W⁵, and W⁶ is independently CH or C(halo); and R⁴, A, R³, R⁵, and R⁶ are each independently as defined in [I-J] or [I-K].

[I-M]

In some embodiments, one of W³ and W⁵ is CR′, and the other of W³ and W⁵ is CH or C(Halo); and each of W² and W⁶ is independently CH or C(halo); and A, R³, R⁵, and R⁶ are each independently as defined in [I-J] through [I-L]; and R⁴ is, e.g., H or C₁-C₆ alkoxy (e.g., OCH₃).

[I-N]

W², W³, W⁵, and W⁶ are defined according to definition (B) as defined anywhere herein; and R⁴, A, R³, R⁵, and R⁶ are each independently as defined in [I-J] or [I-M].

[I-O]

In some embodiments:
each of W², W³, W⁵, and W⁶ is CH;
R⁴ is —CO₂H; —C(O)OR⁴¹; —C(O)N(R⁴²)(R⁴³); —SO₂(R⁴⁵), or heterocyclyloxy;
A is CH₂;
R³, R⁵, and R⁶ are each independently as defined in [I-J] or [I-N].

Embodiments [I-A] through [I-O] can further include any one or more of the features described herein.

Compound Forms and Salts

In some embodiments, the compounds described herein may contain one or more asymmetric centers and thus occur as racemates and racemic mixtures, enantiomerically enriched mixtures, single enantiomers, individual diastereomers and diastereomeric mixtures (e.g., including (R)- and (S)-enantiomers, diastereomers, (D)-isomers, (L)-isomers, (+) (dextrorotatory) forms, (−) (levorotatory) forms, the racemic mixtures thereof, and other mixtures thereof). Additional asymmetric carbon atoms may be present in a substituent, such as an alkyl group. All such isomeric forms, as well as mixtures thereof, of these compounds are expressly included in the present invention. The compounds described herein may also or further contain linkages wherein bond rotation is restricted about that particular linkage, e.g. restriction resulting from the presence of a ring or double bond (e.g., carbon-carbon bonds, carbon-nitrogen bonds such as amide bonds). Accordingly, all cis/trans and E/Z isomers and rotational isomers are expressly included in the present invention. The compounds of this invention may also be represented in multiple tautomeric forms; in such instances, the invention expressly includes all tautomeric forms of the compounds described herein, even though only a single tautomeric form may be represented. All such isomeric forms of such compounds are expressly included in the present invention. Unless otherwise mentioned or indicated, the chemical designation of a compound encompasses the mixture of all possible stereochemically isomeric forms of that compound.

In certain embodiments, the present invention relates to a compound represented by any of the structures outlined herein, wherein the compound is a single stereoisomer. In embodiments, a particular stereoisomer can be substantially free of (e.g., contains less than about 5% of, less than about 2% of, less than about 1%, less than about 0.5% of) another isomer, e.g., its opposing enantiomer and/or one or more other diastereomers.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972), each of which is incorporated herein by reference in their entireties. It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

In embodiments, the compounds described herein may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

The compounds of this invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include C₁₋₆ alkyl esters of carboxylic acid groups, which, upon administration to a subject, are capable of providing active compounds.

Pharmaceutically acceptable salts of the compounds of this invention include those derived from pharmaceutically acceptable inorganic and organic acids and bases. As used herein, the term “pharmaceutically acceptable salt” refers to a salt formed by the addition of a pharmaceutically acceptable acid or base to a compound disclosed herein. As used herein, the phrase “pharmaceutically acceptable” refers to a substance that is acceptable for use in pharmaceutical applications from a toxicological perspective and does not adversely interact with the active ingredient.

Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization. Salt forms of the compounds of any of the formulae herein can be amino acid salts of carboxy groups (e.g. L-arginine, -lysine, -histidine salts).

Lists of suitable salts are found in Remington's Pharmaceutical Sciences, 17th ed., Mack Publishing Company, Easton, Pa., 1985, p. 1418; Journal of Pharmaceutical Science, 66, 2 (1977); “Pharmaceutical Salts: Properties, Selection, and Use A Handbook; Wermuth, C. G. and Stahl, P. H. (eds.) Verlag Helvetica Chimica Acta, Zurich, 2002 [ISBN 3-906390-26-8]; and Berge et al. (1977) “Pharmaceutical Salts”, J. Pharm. Sci. 66:1-19; each of which is incorporated herein by reference in its entirety.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the invention.

In addition to salt forms, the invention provides compounds which are in a prodrug form. Prodrugs of the compounds described herein are those compounds that undergo chemical changes under physiological conditions to provide the compounds of the invention. Additionally, prodrugs can be converted to the compounds of the invention by chemical or biochemical methods in an ex vivo environment. For example, prodrugs can be slowly converted to the compounds of the invention when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the invention which is administered as an ester (the “prodrug”), but then is metabolically hydrolyzed to the carboxylic acid, the active entity. In embodiments, the ester can be an alkyl ester (e.g., C₁-C₃ alkyl, e.g., CH₃ or CH₂CH₃; or C₃-C₆ alkyl, e.g., C₃-C₆ branched alkyl, e.g., t-butyl, isopropyl, isobutyl). Additional examples include peptidyl derivatives of a compound of the invention.

The invention also includes various hydrate and solvate forms of the compounds described herein.

The compounds of the invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the invention, whether radioactive or not, are intended to be encompassed within the scope of the invention.

Synthesis of Compounds of Formula (I)

The compounds described herein can be conveniently prepared in accordance with the procedures outlined in the Examples section, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by one skilled in the art by routine optimization procedures. Those skilled in the art of organic synthesis will recognize that the nature and order of the synthetic steps presented may be varied for the purpose of optimizing the formation of the compounds described herein.

Synthetic chemistry transformations (including protecting group methodologies) useful in synthesizing the compounds described herein are known in the art and include, for example, those such as described in R. C. Larock, Comprehensive Organic Transformations, 2d. ed., Wiley-VCH Publishers (1999); P. G. M. Wuts and T. W. Greene, Protective Groups in Organic Synthesis, 4th Ed., John Wiley and Sons (2007); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The processes described herein can be monitored according to any suitable method known in the art. For example, product formation can be monitored by spectroscopic means, such as nuclear magnetic resonance spectroscopy (e.g., ¹H or ¹³C), infrared spectroscopy (FT-IR), spectrophotometry (e.g., UV-visible), or mass spectrometry (MS), or by chromatography such as high performance liquid chromatograpy (HPLC) or thin layer chromatography (TLC).

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of solvents. Depending on the particular reaction step, suitable solvents for a particular reaction step can be selected.

The compounds of the invention can be prepared, for example, using the reaction pathways and techniques as described below.

In some embodiments, the compounds described herein can be synthesized by the route illustrated in Scheme 1. In STEP 1, the addition of readily available benzensulfonyl chlorides (II) with various readily available substituted amines (III) in the presence of a base (e.g., potassium carbonate or triethyl amine) in either tetrahydrofuran or dichloromethane, respectively, gave the substituted benzenesulfonamide (IV) in good yield. Alkylation of sulfonamide (IV) in STEP 2 is achieved by using substituted benzyl bromides (V) and either potassium carbonate or cesium carbonate (METHOD 1) or via a Mitsunobu reaction using substituted benzyl alcohols (VI) (METHOD 2). The resulting substituted sulfonamides (VII) and (VIII) are isolated in good yields and can be converted to various substituted sulfonamides, such as carboxylic acid derivatives (IX) or sulfone derivatives (X) depending on the aryl substitution (R₃) as depicted in STEP 3, Scheme 1.

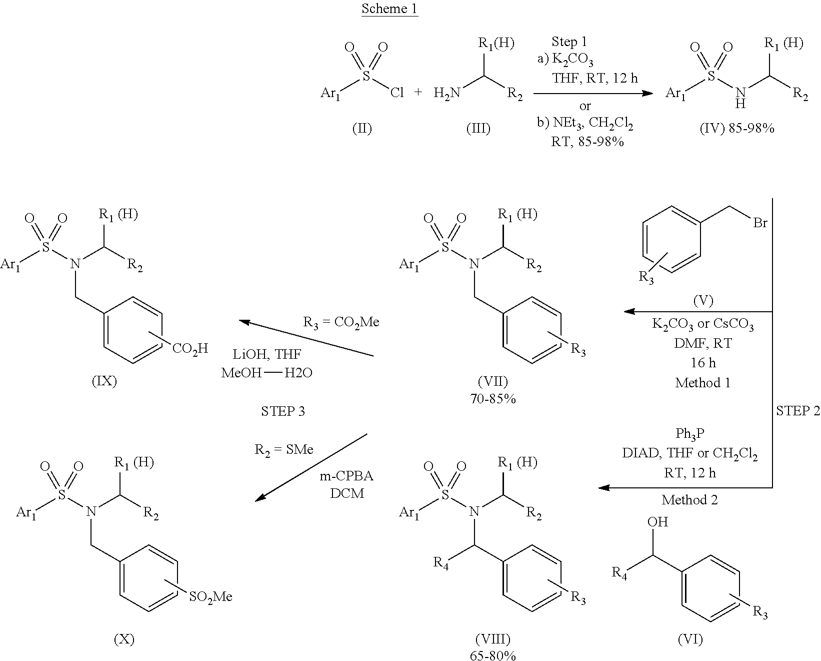

Various substitutions for R₂, of generic structure (1), e.g., Example 42, can be synthesized by the synthetic route illustrated in Scheme 2. This methodology is similar to that depicted in Scheme 1 but employs a different reactant amine (XI) to generate (VII).

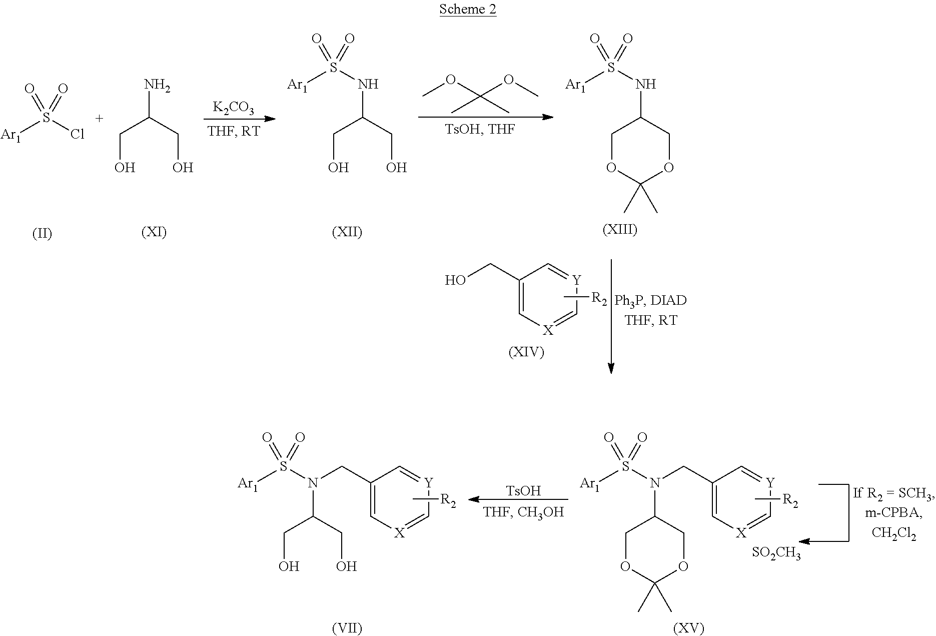

General Method for STEP 1a: (Sulfonylation of Primary Amine)

The solution of amine (III) (10.5 mmol) in 20-25 mL of anhydrous THF was added to potassium carbonate (25 mmol, 2.5 eq) and aryl sulfonyl chloride (II) (10 mmol, 1.0 eq) at room temperature. The reaction mixture was stirred for 16 hrs to completion. The solvent, THF, was removed in vacuo and ethyl acetate was added to extract the crude product. The organic layers were separated and washed with water, brine and dried over sodium sulfate. Subsequent filtration and concentration in vacuo provided the crude sulfonamide which was purified by flash chromatography using 10-50% ethyl acetate in hexane to yield the desired pure sulfonamide (IV).

General Method for STEP 1b: (Sulfonylation of a Primary Amine Salt)

The suspension of the hydrochloric salt of the amine (III) (24 mmol) in anhydrous dichloromethane was added to triethylamine (60 mmol, 2.5 eq) and the aryl sulfonyl chloride (II) (25.2 mmol, 1.05 eq) at room temperature. The reaction mixture was stirred for 2 hrs. Upon completion, 60 mL of 2 N HCl was added. The reaction mixture was then stirred for 25 mins. The precipitated solid was filtered and washed thoroughly with water (5×50 mL) and diethyl ether (5×20 mL). The pure salt of (IV) was dried in a vacuum oven at room temperature.

General Method for STEP 2 (Alkylation of Sulfonamide)

Sulfonamide (IV) can be alkylated either with an aryl bromide (V) (METHOD 1) or with an aryl alcohol (VI) (METHOD 2). For example, to a solution of starting sulfonamide (IV) (0.5 mmol) in 6 mL of THF, Ph₃P (0.6 mmol) and the corresponding benzyl alcohol (VI) (0.6 mmol) were added, followed by DIAD (0.6 mmol). The reaction mixture was stirred at room temperature for 16 hrs. THF was removed in vacuo and the crude residue was purified by flash chromatography using 10-50% ethyl acetate in hexane to yield the alkylated sulfonamide compound (VIII).

Pharmaceutical Compositions, Administration, and Use

The term “pharmaceutically acceptable carrier” refers to a carrier or adjuvant that may be administered to a subject (e.g., a patient), together with a compound of this invention, and which does not destroy the pharmacological activity thereof and is nontoxic when administered in doses sufficient to deliver a therapeutic amount of the compound.

In embodiments, the pharmaceutical compositions described herein may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

In embodiments, pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

In some embodiments, the compounds described herein may contain one or more acidic functional groups and, thus, are capable of forming pharmaceutically-acceptable salts with pharmaceutically-acceptable bases. These salts can be prepared, e.g., in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically-acceptable antioxidants include: water soluble antioxidants, such as ascorbic acid, cysteine hydrochloride, sodium bisulfate, sodium metabisulf[iota]te, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like. In embodiments, formulations of the compounds described herein (and salts thereof) include those suitable for oral, nasal, topical (including buccal and sublingual), rectal, vaginal and/or parenteral administration. The formulations may conveniently be presented in unit dosage form and may be prepared by any conventional methods known in the art of pharmacy. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, this amount will range from about 1% to about 99% of active ingredient, preferably from about 5% to about 70%, most preferably from about 10% to about 30%. In certain embodiments, a formulation of the present invention comprises an excipient selected from the group consisting of cyclodextrins, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention. Methods of preparing these formulations or compositions include the step of bringing into association a compound of the present invention with the carrier and, optionally, one or more accessory ingredients. In general the formulations are prepared by uniformly and intimately bringing into association a compound of the present invention with liquid carriers, or finely divided solid carriers, or both, and then, if necessary, shaping the product. Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent. The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients. Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents. Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound. Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofiuorohydrocarbons and volatile unsubstituted hydrocarbons, such as butane and propane.

Transdermal patches have the added advantage of providing controlled delivery of a compound of the present invention to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin. In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

In certain embodiments, a compound or pharmaceutical preparation is administered orally. In other embodiments, the compound or pharmaceutical preparation is administered intravenously. Alternative routs of administration include sublingual, intramuscular, and transdermal administrations. When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier. The preparations of the present invention may be given orally, parenterally, topically, or rectally. They are of course given in forms suitable for each administration route. For example, they are administered in tablets or capsule form, by injection, inhalation, eye lotion, ointment, suppository, etc., administration by injection, infusion or inhalation; topical by lotion or ointment; and rectal by suppositories. Oral administrations are preferred. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually. Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts. A physician having ordinary skill in the art can readily determine and prescribe the effective amount of the pharmaceutical composition required. For example, the physician could start doses of the compounds of the invention employed in the pharmaceutical composition at levels lower than that required to achieve the desired therapeutic effect and then gradually increasing the dosage until the desired effect is achieved. In some embodiments, a compound or pharmaceutical composition of the invention is chronically provided to a subject with neurodegenerative disorders. Chronic treatments include any form of repeated administration for an extended period of time, such as repeated administrations for one or more months, between a month and a year, one or more years, or longer. In many embodiments, a chronic treatment involves administering a compound or pharmaceutical composition of the invention repeatedly over the life of the subject with neurodegenerative disorders. Preferred chronic treatments involve regular administrations, for example one or more times a day, one or more times a week, or one or more times a month. In general, a suitable dose such as a daily dose of a compound of the invention will be that amount of the compound that is the lowest dose effective to produce a therapeutic effect. Such an effective dose will generally depend upon the factors described above. Generally, doses of the compounds of this invention for a patient, when used for the indicated effects, will range from about 0.0001 to about 100 mg per kg of body weight per day. Preferably the daily dosage will range from 0.001 to 50 mg of compound per kg of body weight, and even more preferably from 0.01 to 10 mg of compound per kg of body weight. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardsley, N.Y., 537 (1970). However, lower or higher doses can be used. In some embodiments, the dose administered to a subject may be modified as the physiology of the subject changes due to age, disease progression, weight, or other factors. If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical formulation (composition) as described above.

In some embodiments, the compounds described herein can be coadministered with one or more other therapeutic agents. In certain embodiments, the additional agents may be administered separately, as part of a multiple dose regimen, from the compounds of this invention (e.g., sequentially, e.g., on different overlapping schedules with the administration of one or more compounds of formula (I) (including any subgenera or specific compounds thereof)). In other embodiments, these agents may be part of a single dosage form, mixed together with the compounds of this invention in a single composition. In still another embodiment, these agents can be given as a separate dose that is administered at about the same time that one or more compounds of formula (I) (including any subgenera or specific compounds thereof) are administered (e.g., simultaneously with the administration of one or more compounds of formula (I) (including any subgenera or specific compounds thereof)). When the compositions of this invention include a combination of a compound of the formulae described herein and one or more additional therapeutic or prophylactic agents, both the compound and the additional agent can be present at dosage levels of between about 1 to 100%, and more preferably between about 5 to 95% of the dosage normally administered in a monotherapy regimen.

The compounds according to the invention may be formulated for administration in any convenient way for use in human or veterinary medicine, by analogy with other pharmaceuticals. According to the invention, compounds for treating neurological conditions or diseases can be formulated or administered using methods that help the compounds cross the blood-brain barrier (BBB). The vertebrate brain (and CNS) has a unique capillary system unlike that in any other organ in the body. The unique capillary system has morphologic characteristics which make up the blood-brain barrier (BBB). The blood-brain barrier acts as a system-wide cellular membrane that separates the brain interstitial space from the blood. The unique morphologic characteristics of the brain capillaries that make up the BBB are: (a) epithelial-like high resistance tight junctions that literally cement all endothelia of brain capillaries together, and (b) scanty pinocytosis or transendothelial channels, which are abundant in endothelia of peripheral organs. Due to the unique characteristics of the blood-brain barrier, hydrophilic drugs and peptides that readily gain access to other tissues in the body are barred from entry into the brain or their rates of entry and/or accumulation in the brain are very low.

In one aspect of the invention, γ-secretase inhibitor compounds that cross the BBB are particularly useful for treating subjects with neurodegenerative disorders. In one embodiment, it is expected that γ-secretase inhibitors that are non-charged (e.g., not positively charged) and/or non-lipophilic may cross the BBB with higher efficiency than charged (e.g., positively charged) and/or lipophilic compounds. Therefore it will be appreciated by a person of ordinary skill in the art that some of the compounds of the invention might readily cross the BBB. Alternatively, the compounds of the invention can be modified, for example, by the addition of various substituents that would make them less hydrophilic and allow them to more readily cross the BBB. Various strategies have been developed for introducing those drugs into the brain which otherwise would not cross the blood-brain barrier. Widely used strategies involve invasive procedures where the drug is delivered directly into the brain. One such procedure is the implantation of a catheter into the ventricular system to bypass the blood-brain barrier and deliver the drug directly to the brain. These procedures have been used in the treatment of brain diseases which have a predilection for the meninges, e.g., leukemic involvement of the brain (U.S. Pat. No. 4,902,505, incorporated herein in its entirety by reference). Although invasive procedures for the direct delivery of drugs to the brain ventricles have experienced some success, they are limited in that they may only distribute the drug to superficial areas of the brain tissues, and not to the structures deep within the brain. Further, the invasive procedures are potentially harmful to the patient.

Other approaches to circumventing the blood-brain barrier utilize pharmacologic-based procedures involving drug latentiation or the conversion of hydrophilic drugs into lipid-soluble drugs. The majority of the latentiation approaches involve blocking the hydroxyl, carboxyl and primary amine groups on the drug to make it more lipid-soluble and therefore more easily able to cross the blood-brain barrier.

Another approach to increasing the permeability of the BBB to drugs involves the intraarterial infusion of hypertonic substances which transiently open the blood-brain barrier to allow passage of hydrophilic drugs. However, hypertonic substances are potentially toxic and may damage the blood-brain barrier.

Peptide compositions of the invention may be administered using chimeric peptides wherein the hydrophilic peptide drug is conjugated to a transportable peptide, capable of crossing the blood-brain barrier by transcytosis at a much higher rate than the hydrophilic peptides alone. Suitable transportable peptides include, but are not limited to, histone, insulin, transferrin, insulin-like growth factor I (IGF-I), insulin-like growth factor II (IGF-II), basic albumin and prolactin.

Antibodies are another method for delivery of compositions of the invention. For example, an antibody that is reactive with a transferrin receptor present on a brain capillary endothelial cell can be conjugated to a neuropharmaceutical agent to produce an antibody-neuropharmaceutical agent conjugate (U.S. Pat. No. 5,004,697 incorporated herein in its entirety by reference). The method is conducted under conditions whereby the antibody binds to the transferrin receptor on the brain capillary endothelial cell and the neuropharmaceutical agent is transferred across the blood brain barrier in a pharmaceutically active form. The uptake or transport of antibodies into the brain can also be greatly increased by cationizing the antibodies to form cationized antibodies having an isoelectric point between 8.0 to 11.0 (U.S. Pat. No. 5,527,527, incorporated herein in its entirety by reference).

A ligand-neuropharmaceutical agent fusion protein is another method useful for delivery of compositions to a host (U.S. Pat. No. 5,977,307, incorporated herein in its entirety by reference). The ligand is reactive with a brain capillary endothelial cell receptor. The method is conducted under conditions whereby the ligand binds to the receptor on a brain capillary endothelial cell and the neuropharmaceutical agent is transferred across the blood brain barrier in a pharmaceutically active form. In some embodiments, a ligand-neuropharmaceutical agent fusion protein, which has both ligand binding and neuropharmaceutical characteristics, can be produced as a contiguous protein by using genetic engineering techniques. Gene constructs can be prepared comprising DNA encoding the ligand fused to DNA encoding the protein, polypeptide or peptide to be delivered across the blood brain barrier. The ligand coding sequence and the agent coding sequence are inserted in the expression vectors in a suitable manner for proper expression of the desired fusion protein. The gene fusion is expressed as a contiguous protein molecule containing both a ligand portion and a neuropharmaceutical agent portion.

The permeability of the blood brain barrier can be increased by administering a blood brain barrier agonist, for example bradykinin (U.S. Pat. No. 5,112,596 incorporated herein in its entirety by reference), or polypeptides called receptor mediated permeabilizers (RMP) (U.S. Pat. No. 5,268,164 incorporated herein in its entirety by reference). Exogenous molecules can be administered to the host's bloodstream parenterally by subcutaneous, intravenous or intramuscular injection or by absorption through a bodily tissue, such as the digestive tract, the respiratory system or the skin. The form in which the molecule is administered (e.g., capsule, tablet, solution, emulsion) depends, at least in part, on the route by which it is administered. The administration of the exogenous molecule to the host's bloodstream and the intravenous injection of the agonist of blood-brain barrier permeability can occur simultaneously or sequentially in time. For example, a therapeutic drug can be administered orally in tablet form while the intravenous administration of an agonist of blood-brain barrier permeability is given later (e.g. between 30 minutes later and several hours later). This allows time for the drug to be absorbed in the gastrointestinal tract and taken up by the bloodstream before the agonist is given to increase the permeability of the blood-brain barrier to the drug. On the other hand, an agonist of blood-brain barrier permeability (e.g. bradykinin) can be administered before or at the same time as an intravenous injection of a drug. Thus, the term “co administration” is used herein to mean that the agonist of blood-brain barrier and the exogenous molecule will be administered at times that will achieve significant concentrations in the blood for producing the simultaneous effects of increasing the permeability of the blood-brain barrier and allowing the maximum passage of the exogenous molecule from the blood to the cells of the central nervous system.

In other embodiments, compounds of the invention can be formulated as a prodrug with a fatty acid carrier (and optionally with another neuroactive drug). The prodrug is stable in the environment of both the stomach and the bloodstream and may be delivered by ingestion. The prodrug passes readily through the blood brain barrier. The prodrug preferably has a brain penetration index of at least two times the brain penetration index of the drug alone. Once in the central nervous system, the prodrug, which preferably is inactive, is hydrolyzed into the fatty acid carrier and the γ-secretase inhibitor (and optionally another drug). The carrier preferably is a normal component of the central nervous system and is inactive and harmless. The compound and/or drug, once released from the fatty acid carrier, is active. Preferably, the fatty acid carrier is a partially-saturated straight chain molecule having between about 16 and 26 carbon atoms, and more preferably 20 and 24 carbon atoms. Examples of fatty acid carriers are provided in U.S. Pat. Nos. 4,939,174; 4,933,324; 5,994,932; 6,107,499; 6,258,836 and 6,407,137, the disclosures of which are incorporated herein by reference in their entirety.

The administration of the agents of the present invention may be for either prophylactic or therapeutic purpose. When provided prophylactically, the agent is provided in advance of disease symptoms such as any Alzheimer's disease symptoms. The prophylactic administration of the agent serves to prevent or reduce the rate of onset of symptoms. When provided therapeutically, the agent is provided at (or shortly after) the onset of the appearance of symptoms of actual disease. In some embodiments, the therapeutic administration of the agent serves to reduce the severity and duration of Alzheimer's disease.

### EXAMPLES

The invention will be further described in the following examples. It should be understood that these examples are for illustrative purposes only and are not to be construed as limiting this invention in any manner.

### Example 1

### (S)-4-((4-Chloro-N-(1-phenylpropyl) phenylsulfonamido) methyl)benzoic acid

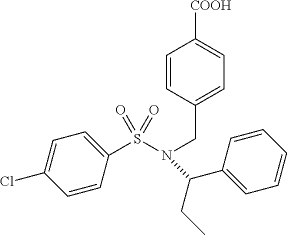

### Step 1

### (S)-4-Chloro-N-(1-phenylpropyl)benzenesulfonamide

A solution of (S)-(−)-α-ethylbenzylamine (500 mg, 3.8 mmol) and potassium carbonate (653 mg, 7.6 mmol) in THF (5 mL) was treated with 4-chlorobenzenesulfonyl chloride (811 mg, 3.8 mmol). After stirring at room temperature for 6 hours, the reaction mixture was concentrated in vacuo and diluted with ethyl acetate and washed with water. The organic phase was separated, dried over magnesium sulfate, filtered and concentrated in vacuo to give the crude product that was recrystallized in hexane and ethyl acetate to afford 4-chloro-N-(1-phenylpropyl)benzenesulfonamide as a white solid (857 mg, 72%). MS (EI⁺) 280.0. Mp 140-142° C.

### Step 2

### (S)-Methyl 4-((4-chloro-N-(1-phenylpropyl) phenylsulfonamido) methyl)benzoate

Method 1

A solution of methyl 4-bromomethylbenzoate (650 mg, 2.8 mmol) and Cs₂CO₃ (1.67 g, 5.13 mmol) in DMF was treated with (S)-4-chloro-N-(1-phenylpropyl)benzenesulfonamide (800 mg, 2.58 mmol). After stirring at room temperature for 6 h, the reaction mixture was filtered. The filtrate was diluted with ethyl acetate and extracted with saturated NaHCO₃ solution and brine (aqueous NaCl). The organic phase was concentrated in vacuo to give an oily residue that was purified by recrystallization with hexane and ethyl acetate to afford (S)-methyl 4-((4-chloro-N-(1-phenylpropyl)phenylsulfonamido)methyl)benzoate, a white solid (837 mg, 71%). MS (m/z) 458.2. Elemental Analysis (C₂₄H₂₄ClNO₄S) Calcd: C, 62.94, H, 5.28, N, 3.06. Found: C, 62.98, H, 5.49, N, 3.14. Mp 108-110° C.

Method 2

To a solution of (S)-4-chloro-N-(1-phenylpropyl)benzenesulfonamide (395 mg, 1.27 mmol), methyl 4-hydroxymethylbenzoate (424 mg, 2.55 mmol), and triphenylphosphine in dichloromethane (3 mL), diisopropylazodicarboxylate (567 mg, 2.8 mmol) was added dropwise at room temperature. After stirring for 5 h, the reaction mixture was diluted with ethyl acetate and washed with saturated NaCl. The organic layer was dried, filtered and concentrated in vacuo to provide an oily mixture that was purified by flash chromatography (ethyl acetate and hexane) to yield (S)-methyl-4-((4-chloro-N-(1-phenylpropyl)phenylsulfonamido)methyl)benzoate as a white solid (380 mg, 65%).

### Step 3

### (S)-4-((4-Chloro-N-(1-phenylpropyl)phenylsulfonamido)methyl)benzoic acid

A solution of (S)-methyl 4-((4-chloro-N-(1-phenylpropyl) phenylsulfonamido)methyl)benzoate (400 mg, 0.875 mmol) in THF (4 mL) was treated with a solution of lithium hydroxide monohydrate in water (2 mL, 2.625 mmol). After stirring for 16 h, the mixture was concentrated in vacuo to give a solution that was acidified with 1N_HCl to pH 3. The resulting white precipitate was filtered and washed with diethyl ether and water and dried to afford (S)-4-((4-chloro-N-(1-phenylpropyl)phenylsulfonamido)methyl)benzoic acid as a white solid (333 mg, 86%). MS (m/z) 444.2. Elemental Analysis (C₂₃H₂₂ClNO₄S) Calcd: C, 62.23, H, 4.99, N, 3.16. Found C, 61.97, H, 4.98, N, 3.07. Mp 177-179° C.

### Example 2

### (S)-4-Chloro-N-(4-(methylsulfonyl)benzyl)-N-(1phenylpropyl)benzenesulfonamide

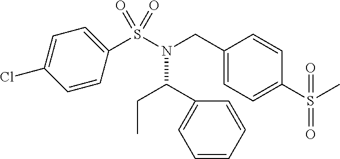

### Step 1

### (S)-4-Chloro-N-(4-(methylthio)benzyl)-N-(1-phenylpropyl)benzenesulfonamide

(S)-4-Chloro-N-(4-(methylthio)benzyl)-N-(1-phenylpropyl)benzenesulfonamide (161 mg, yield 72.2%) was prepared from (S)-4-chloro-N-(1-phenylpropyl)-benzenesulfonamide and 4-(methylthio)benzyl alcohol according to the Method 2 described for STEP 2, Scheme 1. MS (m/z) 446.1 (M⁺+1), Elemental Analysis (C₂₃H₂₄ClNO₂S₂) Calcd: C, 61.94, H, 5.42, N, 3.14. Found: C, 61.83, H, 5.14, N, 3.13.

### Step 2

A solution of (S)-4-chloro-N-(4-(methylthio)benzyl)-N-(1-phenylpropyl)benzenesulfonamide (0.30 mmol) in dichloromethane (DCM) was added m-CPBA (158 mg, 0.90 mmol, 3 eq) and stirred at room temperature for 4 h. DMSO (71 mg, 0.90 mmol, 3 eq) was added to quench the reaction. Saturated Na₂CO₃ aqueous solution was added to adjust the solution to pH 12. DCM was then removed by concentrating in vacuo and the residue was extracted with ethyl acetate. This organic extraction was separated and washed with aqueous Na₂CO₃ solution, water, brine and dried over Na₂SO₄. Subsequent filtration and concentration in vacuo provided the crude product that was purified by flash chromatography with 10-40% ethyl acetate in hexane to yield the title compound (105 mg, 73%). Mp. 61-63° C.; MS (m/z) 478.1 (M⁺+1), Elemental Analysis (C₁₄H₁₄Cl₃NO₂S₂) Calcd: C, 57.79, H, 5.06, N, 2.93. Found: C, 57.91, H, 4.78, N, 2.84.

### Example 3

### (S)-Methyl 4-((4-chloro-N-(1-phenylpropyl)phenylsulfonamido) methyl)benzoate

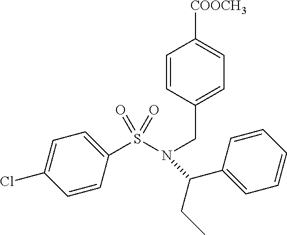

The synthesis of the title compound is described in Example 1, Step 2.

### Example 4

### (S)-Methyl 4-((5-chloro-N-(1-phenylpropyl)thiophene-2-sulfonamido) methyl)benzoate

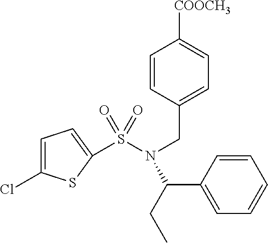

### Step 1

### (S)-5-Chloro-N-(1-phenylpropyl)thiophene-2-sulfonamide

(S)-5-Chloro-N-(1-phenylpropyl)thiophene-2-sulfonamide was prepared from 5-chlorothiophene-2-sulfonyl chloride and (S)-(−)-α-ethylbenzylamine according to the general method illustrated in Scheme 1, Step 1a. Yield: 77%.

### Step 2

### (S)-Methyl 4-((5-chloro-N-(1-phenylpropyl)thiophene-2-sulfonamido)methyl)benzoate

The title compound was prepared from methyl 4-hydroxylmethylbenzoate and (S)-5-chloro-N-(1-phenylpropyl)thiophene-2-sulfonamide according to the general method illustrated in Scheme 1, Step 2, Method 2. Yield: 50%.

Elemental Analysis (C₂₂H₂₂ClNO₄S₂) Calcd: C, 56.95, H, 4.78, N, 3.02. Found: C, 56.71, H, 5.04, N, 3.17. MS (EI⁺) 434.0

### Example 5

### (S)-4-((4-Chloro-N-(1-phenylpropyl)phenylsulfonamido)methyl)-N-(2-hydroxyethyl)benzamide

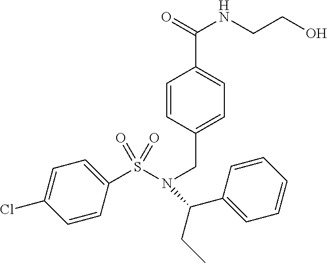 [SMILES: CC[C@@H](c1ccccc1)N(Cc1ccc(C(=O)NCCO)cc1)S(=O)(=O)c1ccc(Cl)cc1]

Method 1

A solution of (S)-methyl 4-((4-chloro-N-(1-phenylpropyl)phenylsulfonamido)methyl)benzoate (200 mg, 0.43 mmol) in ethanolamine (320 mg, 5.24 mmol) was stirred at 110° C. for 1 h. The reaction mixture was then diluted in ethyl acetate and washed with saturated NaCl. The organic layer was separated and concentrated in vacuo. This crude product was purified using a Combiflash system (methanol/dichloromethane) to afford the title compound as a colorless liquid (105 mg, 49%). Elemental Analysis (C₂₅H₂₇ClN₂O₄S) Calcd: C, 61.66, H, 5.59, N, 5.75. Found: C, 61.93, H, 5.54, N, 5.76.

Method 2

A solution of (S)-4-((4-chloro-N-(1-phenylpropyl)phenylsulfonamido)methyl)benzoic acid (150 mg, 0.34 mmol) in dichloromethane was treated with ethanolamine (41 mg), dicyclohexylcarbodiimide (110 mg, 0.54 mmol) and 1-hydroxybenzotriazole (50 mg, 0.37 mmol). The reaction mixture was stirred for 16 h and then diluted with ethyl acetate. The organic layer was washed with a saturated NaCl aqueous solution and then concentrated in vacuo. The crude product was purified using a Combiflash system (methanol/dichloromethane) to afford the title compound as a liquid (68 mg, 29%)

### Example 6

### (S)-4-Chloro-N-(4-(5-methyl-1,3,4-oxadiazol-2-yl)benzyl)-N-(1-phenylpropyl)benzenesulfoamide

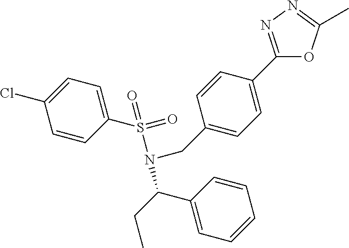 [SMILES: CC[C@@H](c1ccccc1)N(Cc1ccc(-c2nnc(C)o2)cc1)S(=O)(=O)c1ccc(Cl)cc1]

A solution of (S)-methyl 4-((4-chloro-N-(1-phenylpropyl)phenylsulfonamido)methyl)benzoate (288 mg, 0.63 mmol) and hydrazine monohydrate (400 mg, 12 mmol) in methanol (1 mL) was refluxed for 6 h at 70° C. The reaction mixture was concentrated in vacuo to afford crude hydrazine. This hydrazine was then treated with ethyl orthoacetate (443 mg, 2.51 mmol) and p-toluenesulfonic acid monohydrate (38 mg, 0.2 mmol). The mixture was refluxed for 24 h, cooled and then concentrated in vacuo. Purification of the crude product by column chromatography (ethyl acetate/hexane) afforded the title compound (130 mg, 54%) as a liquid. Elemental Analysis (C₂₅H₂₄ClN₃O₃S) Calcd: C, 62.30, H, 5.02, N, 8.72. Found: C, 62.00, H, 5.23, N, 8.44. MS (m/z) 415.2.

### Example 7

### (R)-Methyl 4-((4-chloro-N-(2-hydroxy-1-phenylethyl)phenylsulfonamido)methyl)benzoate

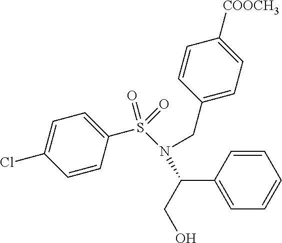 [SMILES: COC(=O)c1ccc(CN([C@@H](CO)c2ccccc2)S(=O)(=O)c2ccc(Cl)cc2)cc1]

### Step 1

### (R)-4-Chloro-N-(2-hydroxy-1-phenylethyl)benzenesulfonamide

To a mixture of (R)-2-amino-2-phenylethanol (1.0 g, 7.29 mmol, 1 eq) and Et₃N (2.5 eq) in a 100 mL round-bottomed flask in dichloromethane (15 mL), 4-chlorobenzenesulfonyl chloride (1.54 g, 7.29 mmol) in 5 mL dichloromethane was added. The reaction was stirred at room temperature for 3 h and then 30 mL of water was added. The two phases were separated and the aqueous phase was extracted with dichloromethane. The combined organic extracts were dried over Na₂SO₄ and concentrated in vacuo to give a crude residue that was recrystallized using ethyl acetate and hexane to yield the product as a white solid (4.65 g, 63%). MS (m/z) EI⁺280 (M⁺-CH₂OH). Elemental Analysis: Calcd: C, 53.93, H, 4.53, N, 4.49. Found: C, 54.09, H, 4.27, N, 4.48.

### Step 2

### (R)-2-(4-Chlorophenylsulfonamido)-2-phenylethyl acetate

(R)-4-Chloro-N-(2-hydroxy-1-phenylethyl)benzenesulfonamide (600 mg, 1.92 mmol) was refluxed in acetic anhydride (1.18 g, 11.5 mmol) for 50 min at 95° C. The reaction mixture was then cooled, diluted with ethyl acetate and washed with water and saturated NaHCO₃. The organic layers were separated and dried over Na₂SO₄ to yield a crude white solid. Recrystallization of the solid using hexane and ethyl acetate afforded a white solid product (620 mg, 91%). MS (m/z) 280 (M⁺−73). Elemental Analysis: Calcd: C, 54.31; H, 4.56; N, 3.96. Found: C, 54.49; H, 4.45; N, 3.95.

### Step 3

### (R)-Methyl 4-0N-(2-acetoxy-1-phenylethyl)-4-chlorophenylsulfonamido) methyl)benzoate

To a solution of (R)-2-(4-chlorophenylsulfonamido)-2-phenylethyl acetate (500 mg, 1.41 mmol), methyl 4-hydroxymethylbenzoate (470 mg, 2.82 mmol) and triphenylphosphine (852 mg, 3.25 mmol) in dichloromethane (10 mL), diisopropylazodicarboxylate (629 mg, 3.1 mmol) was added dropwise at room temperature. After stirring for 4 h, the reaction mixture was diluted with ethyl acetate and washed with saturated NaCl. The organic layer was dried, concentrated in vacuo and filtered to provide a mixture that was purified by flash chromatography (ethyl acetate and hexane) to yield (R)-methyl 4-((N-(2-acetoxy-1-phenylethyl)-4-chlorophenylsulfonamido)methyl)benzoate (490 mg, 69%). MS (m/z) 453.2 (M⁺-CH₃O₂).

### Step 4

### (R)-Methyl-4-((4-chloro-N-(2-hydroxy-1-phenylethyl)phenylsulfonamido) methyl)benzoate

A solution of (R)-methyl 4-((N-(2-acetoxy-1-phenylethyl)-4-chlorophenylsulfonamido)methyl)benzoate (100 mg, 0.199 mmol) in methanol (1 mL) was added sodium methoxide (6 mg). The reaction was stirred for 1 hr and then concentrated in vacuo. The crude product was purified with flash chromatography (ethyl acetate and hexane) to afford the title compound (62 mg, 67%). Elemental Analysis (C₂₀H₂₄FNO₄S) Calcd: C, 60.06, H, 4.82, N, 3.05. Found: C, 59.95, H, 4.74, N, 2.93. MS (EI⁺): 460.1. Mp 104-106° C.

### Example 8

### (S)-4-Chloro-N-(4-(hydroxymethyl)benzyl)-N-(1-phenylpropyl)benzenesulfonamide

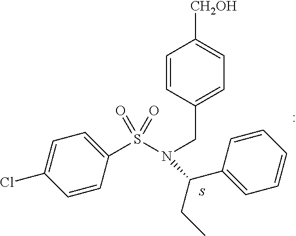

A solution of (S)-methyl 4-((4-chloro-N-(1-phenylpropyl)phenylsulfonamido)methyl)benzoate (100 mg, 0.218 mmol) in THF (4 mL) was treated with lithium aluminum tetrahydride (0.393 mmol). The reaction mixture was stirred for 1 h and then treated twice with 0.5 mL water and 4 N NaOH until the mixture reached pH 9. The mixture was then stirred for 15 minutes and then concentrated in vacuo. The residue was then extracted with ethyl acetate and the organic layer was concentrated in vacuo. The residue was purified by flash chromatography (ethyl acetate and hexane) to afford the title compound (62 mg, 87%). Elemental Analysis (C₂₃H₂₄ClNO₃S) Calcd. C, 64.25, H, 5.63, N, 3.26. Found 64.53, H, 5.04, N, 3.17. MS (EI⁺) 400.1.

### Example 9

### (R)-4-((4-Chloro-N-(2-hydroxy-1-phenylethyl)phenylsulfonamido)methyl)-N-(2-hydroxyethyl)benzamide

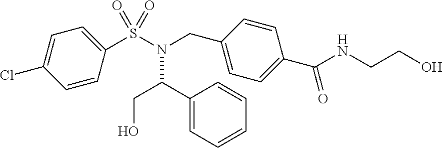

A solution of (R)-methyl 4-((N-(2-acetoxy-1-phenylethyl)-4-chlorophenylsulfonamido)methyl)benzoate (100 mg, 1.99 mmol) in ethanolamine (0.8 mL) was stirred for 4 h at 105° C. The reaction mixture was diluted with ethyl acetate and washed with water and saturated NaCl. The organic layers were separated, concentrated in vacuo, and purified via flash chromatrography (acetone and ethyl acetate) to provide the title compound as a liquid (45 mg, 46%). MS (m/z) 489.3.

### Example 10

### (S)-4-((4-Chloro-N-(1-phenylpropyl)phenylsulfonamido)methyl)-N-(2-hydroxyethyl)benzamide

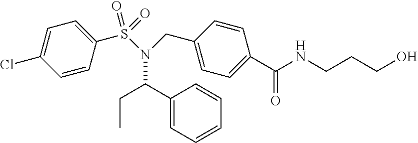

A solution of (S)-methyl 4-((4-chloro-N-(1-phenylpropyl)phenylsulfonamido)methyl)benzoate (200 mg, 0.436 mmol) and 1-hydroxypropanol (0.8 mL) was stirred for 3 h at 120° C. The reaction mixture was diluted with ethyl acetate and washed with water and saturated NaCl. The organic layer was separated, concentrated in vacuo and purified via flash chromatography (acetone and ethyl acetate) to provide the title compound as a liquid (126 mg, 58%). MS (EI⁺) 501.1. Elemental Analysis (C₂₄H₂₅ClN₂O₄S):: Calcd: C, 62.33, H, 5.83, N, 5.59. Found: C, 62.09, H, 5.74, N, 5.59.

### Example 11

### 4-((4-Chloro-N-((S)-1-phenylpropyl)phenylsulfonamido)methyl)-N-((R)-1-hydroxypropan-2-yl)benzamide

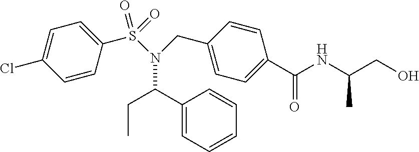

A solution of (S)-methyl 4-((4-chloro-N-(1-phenylpropyl)phenylsulfonamido)methyl)benzoate (200 mg, 1.99 mmol) in (R)-2-aminopropanol (0.6 mL) was stirred for 4 h at 135° C. The reaction mixture was diluted with ethyl acetate and washed with water and saturated NaCl. The organic layer was separated, concentrated in vacuo and purified via flash chromatography (acetone and ethyl acetate) to provide the title compound as a liquid (143 mg, 65%). MS (m/z): 501.04.

### Example 12

### (S)-Methyl 4-((4-chloro-N-(1-phenylethyl)phenylsulfonamido)methyl)benzoate

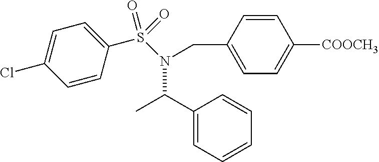

### Step 1

### (S)-4-Chloro-N-(1-phenylethyl)benzenesulfonamide

(S)-4-Chloro-N-(1-phenylethyl)benzenesulfonamide was prepared from 4-chlorophenyl sulfonyl chloride and (S)-1-methylbenzylamine according to the general method illustrated in Scheme 1, Step 1. Yield: 76%.

### Step 2

### (S)-Methyl 4-((4-chloro-N-(1-phenylethyl)phenylsulfonamido)methyl)benzoate

(S)-Methyl 4-((4-chloro-N-(1-phenylethyl)phenylsulfonamido)methyl)benzoate was prepared from 4-chloro-N-(1-phenylethyl)benzenesulfonamide and 4-hydroxybenzoate according to the general method illustrated in Scheme 1, STEP 2, Method 2. Yield: 61%. Mp 105-106° C. MS (m/z) 440.0. Elemental Analysis (C₂₃H₂₂ClNO₄S) Calcd: C, 62.23; H, 4.99; N, 3.16. Found: C, 62.22; H, 5.00; N, 3.16.

### Example 13

### 4-((4-Chloro-N-((R)-2-hydroxy-1-phenylethyl)phenylsulfonamido)methyl)-N-((S)-1-hydroxypropan-2-yl)benzamide

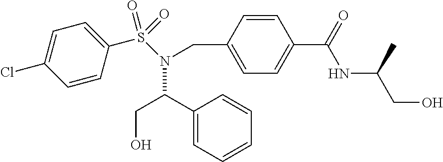

A solution of (R)-methyl 4-((N-(2-acetoxy-1-phenylethyl)-4-chlorophenylsulfonamido)methyl)benzoate (120 mg, 0.239 mmol) in (S)-2-amino-1-propanol (0.3 mL) was stirred for 5 h at 120° C. The reaction mixture was diluted with ethyl acetate and washed with water and saturated NaCl. The organic layer was separated and concentrated in vacuo and was purified via flash chromatography (acetone and ethyl acetate) to provide the title compound (40 mg, 33%). MS⁺ (m/z) 503.1 Elemental Analysis (C₂₃H₂₂ClNO₄S) Calcd: C, 59.69, H, 5.41, N, 5.57. Found: C, 59.97, H, 5.77, N, 5.34.

### Example 14

### 4-((4-Chloro-N-((S)-1-phenylpropyl)phenylsulfonamido)methyl)-N-(2-hydroxypropyl)benzamide

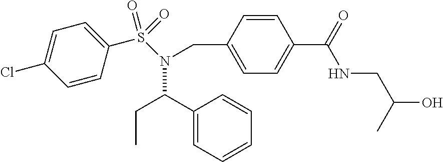

The solution of (S)-methyl 4-((4-chloro-N-(1-phenylpropyl) phenylsulfonamido)methyl)benzoate in 3-amino-2-propanol (0.3 mL) was stirred for 5 h at 140° C. The reaction mixture was diluted with ethyl acetate and washed with water and saturated NaCl. The organic separated was concentrated in vacuo and purified via flash chromatography (acetone and ethyl acetate) to provide the title compound (129 mg, 74%). MS⁺ (m/z) 471.2. Elemental Analysis (C₂₆H₂₉ClN₂O₄S) Calcd: C, 62.33, H, 5.83, N, 5.59. Found: C, 62.09, H, 6.01, N, 5.31.

### Example 15

### (S)-4-((4-Chloro-N-(1-phenylpropyl)phenylsulfonamido)methyl)-N-(1-hydroxy-2-methylpropan-2-yl)benzamide

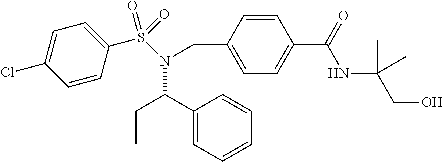

The solution of (S)-methyl 4-((4-chloro-N-(1-phenylpropyl) phenylsulfonamido)methyl)benzoate (160 mg, 0.35 mmol) in 2-amino-2-methyl-1-propanol (0.3 mL) was stirred for 13 h at 140° C. The reaction mixture was diluted with ethyl acetate and washed with water and saturated NaCl. The organic layer was separated and concentrated in vacuo, and was purified via flash chromatography (acetone and acetate) to provide the title compound (40 mg, 22%). MS⁺ (m/z) 515.2. Elemental Analysis (C₂₇H₃₁ClN₂O₄S) Calcd: C, 62.96, H, 6.07, N, 5.44. Found: C, 62.71, H, 5.91, N, 5.34.

### Example 16

### (S)-4-((4-Chloro-N-(1-phenylpropyl)phenylsulfonamido)methyl)benzamide

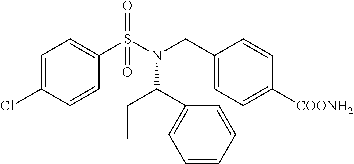

The title compound was prepared from 4-chloro-N-(1-phenylpropyl)benzenesulfonamide and 4-(bromomethyl)benzamide according to the general method illustrated in Scheme 1, STEP 2, Method 1. Yield: 34%. Mp 158-159° C. MS (m/z) 413.0. Elemental Analysis (C₂₃H₂₃ClN₂O₄S) Calcd: C, 62.36, H, 5.23; N, 6.32. Found: C, 60.90, H, 4.94, N, 6.54.

### Example 17

### (S)-Methyl 4-((4-chloro-N-(1-(4-fluorophenyl)propyl)phenylsulfonamido)methyl)benzoate

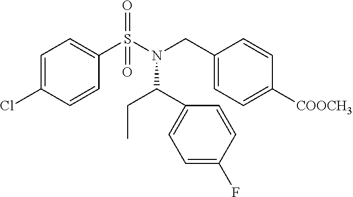

### Step 1

### (S)-4-Chloro-N-(1-(4-fluorophenyl)propyl)benzenesulfonamide

(S)-4-Chloro-N-(1-(4-fluorophenyl)propyl)benzenesulfonamide was prepared from 4-chlorophenylsulfonyl chloride and (S)-1-(4-fluorophenyl)-1-propanylamine according to the general method illustrated in Scheme 1, STEP 1a. Yield: 84%.

### Step 2

### (S)-Methyl 4-((4-chloro-N-(1-(4 fluorophenyl)propyl)phenylsulfonamido)methyl)benzoate

(S)-Methyl 4-((4-chloro-N-(1-(4-fluorophenyl)propyl)phenylsulfonamido)methyl)benzoate was prepared from (S)-4-chloro-N-(1-(4-fluorophenyl)propyl)benzenesulfonamide and methyl 4-bromomethyl benzoate according to the general method illustrated in Scheme 1, STEP 2, Method 1. Yield: 80%. Mp 73-75° C. MS⁺ (m/z) 445.9. Elemental Analysis (C₂₃H₂₃ClFNO₄S) Calcd: C, 60.56, H, 4.87, N, 2.94. Found: C, 60.51, H, 4.57, N, 3.07.

### Example 18

### (S)-Methyl 4-((N-(1-phenylpropyl)-4-(trifluoromethyl)phenylsulfonamido)methyl)benzoate

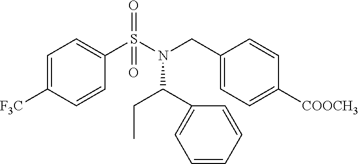

### Step 1

### (S)-4-Trifluoromethyl-N-(1-phenylpropyl)benzenesulfonamide

(S)-4-Trifluoromethyl-N-(1-phenylpropyl)benzenesulfonamide was prepared from 4-trifluoromethylphenyl sulfonyl chloride and (S)-1-ethylbenzylamine according to the general method illustrated in Scheme 1, STEP 1. Yield: 82%.

### Step 2

### (S)-Methyl 4-((N-(1-phenylpropyl)-4-(trifluoromethyl)phenylsulfonamido)methyl)benzoate

(S)-Methyl 4-((N-(1-phenylpropyl)-4-(trifluoromethyl)phenylsulfonamido)methyl)benzoate was prepared from (S)-4-trifluoromethyl-N-(1-phenylpropyl)benzenesulfonamide and methyl 4-bromomethyl benzoate according to the general method illustrated in Scheme 1, STEP 2, Method 1. Yield: 61%. Mp 70-72° C. MS (m/z) 461.9. Elemental Analysis (C₂₅H₂₄F₃NO₄S) Calcd: C, 60.09, H, 4.92, N, 2.85. Found: C, 60.16, H, 4.58, N, 2.85.

### Example 19

(S)-4-Chloro-N-(4-methoxybenzyl)-N-(1-phenylpropyl)benzenesulfonamide

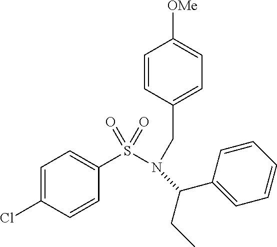

The title compound was prepared from (S)-4-chloro-N-(1-phenylpropyl)benzenesulfonamide and 4-methoxybenzyl alcohol according to Method 2 illustrated in Scheme 1, STEP 2. Yield: 53%. MS (m/z) 430.2. Elemental Analysis (C₂₃H₂₄ClNO₃S) Calcd: C, 64.25, H, 5.63, N, 3.26. Found: C, 64.53, H, 5.49, N, 3.01.

### Example 20

### (S)-4-chloro-N-(1-phenylpropyl)-N-(4-(trifluoromethyl)benzyl)benzenesulfonamide

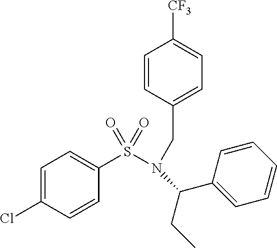 [SMILES: CC[C@@H](c1ccccc1)N(Cc1ccc(C(F)(F)F)cc1)S(=O)(=O)c1ccc(Cl)cc1]

The title compound was prepared from (S)-4-chloro-N-(1-phenylpropyl)benzenesulfonamide and 4-trifuromethylbenzyl alcohol according to the general method illustrated in Scheme 1, STEP 2, Method 2. Yield: 49%. MS⁺ (m/z) 468.2. Elemental Analysis for C₂₃H₂₁ClF₃NO₂S: Calcd: C, 59.04, H, 4.52, N, 2.99. Found: C, 59.32, H, 4.24, N, 3.11.

### Example 21

### (S)-Methyl 4-((4-fluoro-N-(1-phenylpropyl)phenylsulfonamido) methyl)benzoate

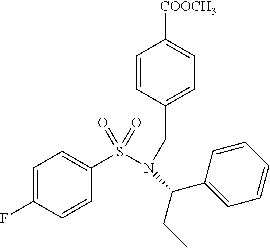 [SMILES: CC[C@@H](c1ccccc1)N(Cc1ccc(C(=O)OC)cc1)S(=O)(=O)c1ccc(F)cc1]

### Step 1

### (S)-4-Fluoro-N-(1-phenylpropyl)benzenesulfonamide

(S)-4-Fluoro-N-(1-phenylpropyl)benzenesulfonamide was prepared from 4-fluorophenylsulfonyl chloride and (S)-1-ethylbenzylamine according to the general method illustrated in Scheme 1, STEP 1a. Yield: 72%.

### Step 2

### (S)-Methyl 4-((4-fluoro-N-(1-phenylpropyl)phenylsulfonamido)methyl)benzoate

(S)-Methyl 4-((4-fluoro-N-(1-phenylpropyl)phenylsulfonamido)methyl)benzoate was prepared from (S)-4-fluoro-N-(1-phenylpropyl)benzenesulfonamide and methyl 4-hydroxylmethylbenzoate according to the general method illustrated in Scheme 1, STEP 2, Method 2. Yield: 61%. Mp 107-109° C. MS+ (m/z) 442.2. Elemental Analysis for C₂₄H₂₄FNO₄S: Calcd: C, 65.29, H, 5.48, N, 3.17. Found: C, 64.58, H, 5.28, N, 3.10.

### Example 22

### (S)-4-Chloro-N-(4-(4,5-dihydrooxazol-2-yl)benzyl)-N-(1-phenylpropyl)benzenesulfonamide

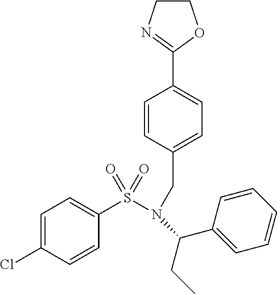 [SMILES: CC[C@@H](c1ccccc1)N(Cc1ccc(C2=NCCO2)cc1)S(=O)(=O)c1ccc(Cl)cc1]

To a solution of (S)-4-((4-chloro-N-(1-phenylpropyl)phenylsulfonamido)methyl)-N-(2-hydroxyethyl)benzamide (90 mg, 0.185 mmol) in dichloromethane (2 mL) was added diethylaminosulfur (36 mg, 1.2 eq) and potassium carbonate (51 mg, 2 eq) at −78° C. The reaction was warmed to room temperature and stirred for 6 h. The reaction mixture was washed with water and the organic layer was dried over Na₂SO₄, filtered, and concentrated under reduced pressure. The residue was purified by silica gel column chromatography (5% methanol in dichloromethane) to yield the title compound as an oil (25 mg, 43%). MS⁺ (m/z) 439.0. Elemental Analysis (C₂₅H₂₃ClN₂O₃S) Calcd: C, 64.02, H, 5.37, N, 5.97. Found: C, 63.77, H, 5.37, N, 5.97.

### Example 23

(S)-4-((4-Chloro-N-(1-phenylpropyl)phenylsulfonamido)methyl)-3-methoxybenzoic acid

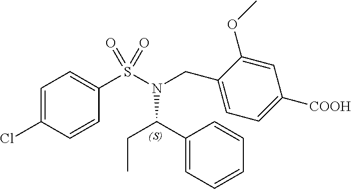

### Step 1

### (S)-Methyl 4-((4-chloro-N-(1-phenylpropyl)phenylsulfonamido)methyl)-3-methoxybenzoate

The mixture of (S)-4-chloro-N-(1-phenylpropyl)benzenesulfonamide (309 mg, 0.997 mmol) and methyl 4-(bromomethyl)-3-methoxybenzoate (279 mg, 1.077 mmol) in 3 mL of DMF was added Cs₂CO₃. The reaction mixture was stirred at room temperature for 16 h. Water (12 mL) was then added to the reaction and the reaction mixture was then extracted with ethyl acetate. The organic layer was separated and washed with water, brine and dried over sodium sulfate. Filtration and removal of solvent in vacuo provided 473 mg of white solid which was purified by combiflash chromatography (0-30% hexane and ethyl acetate). Desired product as a white solid was isolated (415 mg) Mp 108-110° C.; MS m/z 488; Elemental Analysis (C₂₅H₂₆ClNO₅S) Calcd: C, 61.53, H, 5.37, N, 2.87. Found: C, 61.71, H, 5.25, N, 2.62.

### Step 2

### (S)-4-((4-chloro-N-(1-phenylpropyl)phenylsulfonamido)methyl)-3-methoxybenzoic acid

The solution of 200 mg of the above product [(S)-Methyl 4-((4-chloro-N-(1-phenylpropyl)-phenylsulfonamido)methyl)-3-methoxybenzoate] was dissolved in 4 mL THF and was added 0.5 mL of methanol and 0.5 mL of water. The solution was then added LiOH hydrate and was stirred at 50° C. Reaction was then monitored by TLC (Hexane/EA=1:2). After 6 h heating and stirring, reaction is completed. THF and methanol was removed and 1.5 mL of water was added to the residue. Then, 2 N HCl was added and the reaction mixture to bring pH to 2. White precipitate formed. The solid was filtered and washed with water, and hexane and then was dried in a vacuum oven. After drying, 162 mg of white solid was collected (83%). Mp 176-178° C. MS (m/zi) 474 (M⁺+1). Elemental Analysis (C₂₄H₂₄ClNO₅S) Calcd: C, 60.82, H, 5.10, N, 2.96. Found: C, 60.70, H, 5.12, N, 2.85.

### Example 24

(S)-Methyl 4-((4-fluoro-N-(1-(4-fluorophenyl)ethyl) phenylsulfonamido) methyl)benzoate

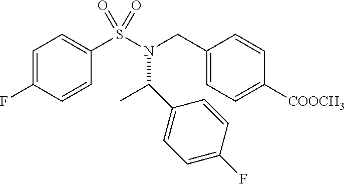

### Step 1

### Ethyl 4-((4-fluorophenylsulfonamido)methyl)benzoate

Ethyl 4-((4-fluorophenylsulfonamido)methyl)benzoate (6.48 g, 82.4%, white solid) was prepared from 4-fluorophenyl sulfonyl chloride and methyl 4-(aminomethyl)benzoate hydrochloride according to procedure described in STEP 1, Scheme 1.

### Step 2

### (S)-Methyl 4-((4-fluoro-N-(1-(4-fluorophenyl)ethyl)phenylsulfonamido) methyl)benzoate

(S)-Methyl 4-((4-fluoro-N-(1-(4-fluorophenyl)ethyl)phenylsulfonamido) methyl)benzoate (397 mg, 89.1%) was prepared from methyl 4-((4-fluorophenylsulfonamido) methyl)benzoate and (R)-4-fluoro-α-methylbenzyl alcohol according to the general method described in STEP 2, Scheme 1. MS (m/z) 446.1 (M⁺+1). Elemental Analysis (C₂₃H₂₁F₂NO₄S) Calcd: C, 62.01, H, 4.75, N, 3.14. Found: C, 61.75, H, 4.93, N, 3.10.

### Example 25

### Methyl 4-((4-chloro-N-(1-phenylpropyl)phenylsulfonamido)methyl)benzoate

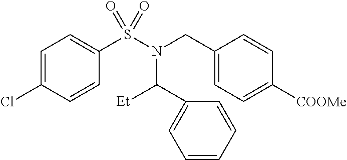 [SMILES: CCC(c1ccccc1)N(Cc1ccc(C(=O)OC)cc1)S(=O)(=O)c1ccc(Cl)cc1]

### Step 1

### 4-((4-Chlorophenylsulfonamido)methyl)benzoate

To a mixture of methyl 4-(aminomethyl)benzoate hydrochloride (4.16 g, 20 mmol, 1.0 eq) and Et₃N (7 mL, 50 mmol, 2.5 eq) in a 100 mL round-bottomed flask in dichloromethane (50 mL), 4-chlorobenzenesulfonyl chloride (4.35 g 20 mmol, dissolved in 20 mL dichloromethane) was added over 10 minutes via syringe at room temperature. After stirring for 1 h, 100 mL of water was added and the two phases were separated. The aqueous phase was extracted with dichloromethane and the combined organic extracts were dried over Na₂SO₄ and concentrated in vacuo to give a crude residue. Diethyl ether (100 mL) was added to the residue and the mixture was then stirred at 40° C. for 10 minutes and filtered to yield 4-((4-chlorophenylsulfonamido)methyl)benzoate, a white solid product (5.77 g, 85%).

### Step 2

### Methyl 4-((4-chloro-N-(1-phenylpropyl)phenylsulfonamido)methyl)benzoate

To a solution of methyl 4-((4-chlorophenylsulfonamido)methyl)benzoate (152 mg, 0.44 mmol), 1-phenylpropan-1-ol (120.0 mg, 0.88 mmol) and Ph₃P (255.0 mg, 0.97 mmol) in 5 mL of THF, diisopropyl azodicarboxylate (202 μL, 0.97 mmol) was added dropwise. The light yellow mixture was stirred at room temperature for 16 h. Water (40 mL) was then added to the reaction, and the mixture was then extracted with ethyl acetate and concentrated in vacuo. The residue was purified using 15% ethyl acetate in hexane to yield the title compound (78%), methyl 4-((4-chloro-N-(1-phenylpropyl) phenylsulfonamido)methyl)benzoate. Elemental Analysis (C₂₄H₂₄ClNO₄S) Calcd: C, 62.94, H, 5.28, N, 3.06. Found: C, 63.21, H, 5.27, N, 3.06. Mp 105-107° C.

### Example 26

Methyl 4-((4-fluoro-N-(1-(pyridin-2-yl)propyl)phenylsulfonamido)methyl)benzoate

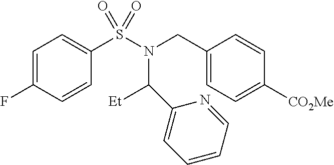 [SMILES: CCC(c1ccccn1)N(Cc1ccc(C(=O)OC)cc1)S(=O)(=O)c1ccc(F)cc1]

### Step 1

### Methyl 4-((4-fluorophenylsulfonamido)methyl)benzoate

To a mixture of methyl 4-(aminomethyl)benzoate hydrochloride and triethyl amine in dichloromethane (DCM) (30 mL), 4-fluorobenzenesulfonyl chloride in 20 mL of DCM was added over 10 minutes via syringe. After stirring for 1 h, 100 mL of water was added and then extracted with DCM. The combined organic extracts were dried over Na₂SO₄ and concentrated in vacuo. Diethyl ether (100 mL) was then added to the residue and the mixture was stirred at 40° C. for 10 minutes. A white solid precipitated that was filtered and dried to yield the desired product, methyl 4-((4-fluorophenylsulfonamido)methyl)benzoate (50 g, 85%).

### Step 2

To a solution of methyl 4-((4-fluorophenylsulfonamido)methyl)benzoate (162 mg, 0.5 mmol, 1.0 eq), 1-(pyridin-2-yl)propan-1-ol (140 mg, 1.0 mmol, 2.0 eq) and Ph₃P (292 mg, 1.1 mmol, 2.2 eq) in 5 mL of THF, diisopropyl azodicarboxylate (DIAD) (228 μL, 1.1 mmol, 2.2 eq) was added dropwise. The light yellow mixture was stirred at room temperature for 16 h. Water (40 mL) was then added and the mixture was extracted with EtOAc. The combined organic extracts were dried over Na₂SO₄ and concentrated in vacuo to yield a crude product that was purified by column chromatography with 20% ethyl acetate in hexane to give the title compound as a white solid. (139.0 mg, 71% yield). Mp 82-84° C. Elemental Analysis (C₂₃H₂₃FN₂O₄S) Calcd: C, 62.43, H, 5.24, N, 6.33. Found: C, 62.45, H, 5.52, N, 6.36.

### Example 27

### 4-((4-Chloro-N-(1-phenylpropyl)phenylsulfonamido)methyl)benzoic acid

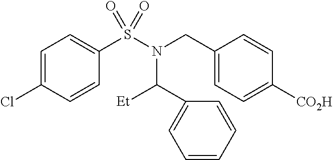 [SMILES: CCC(c1ccccc1)N(Cc1ccc(C(=O)O)cc1)S(=O)(=O)c1ccc(Cl)cc1]

### Step 1

### Methyl 4-((4-chlorophenylsulfonamido) methyl)benzoate

To a mixture of methyl 4-(aminomethyl)benzoate hydrochloride and Et₃N in dichloromethane (DCM) (30 mL) was added 4-chlorobenzenesulfonyl chloride in 20 mL of DCM over 10 minutes using a syringe. After stirring for 1 h, 100 mL of water was added and the mixture was then extracted with DCM. The combined organic extracts were dried over Na₂SO₄, concentrated in vacuo, and then 100 mL of diethyl ether was then added and the mixture was stirred at 40° C. for 10 minutes. The white precipitate was then filtered and dried to give methyl 4-((4-chlorophenylsulfonamido) methyl)benzoate (7.5 g, 90%)

### Step 2

To a solution of methyl 4-((4-fluorophenylsulfonamido)methyl)benzoate (162 mg, 05. mmol, 1.0 eq), 1-phenylpropan-1-ol (138 mg, 1.0 mmol, 2.0 eq) and Ph₃P (292 mg, 1.1 mmol, 2.2 eq) in 5 mL of THF, diisopropyl azodicarboxylate (DIAD) (228 μL, 1.1 mmol, 2.2 eq) was added dropwise. The light yellow mixture was stirred at room temperature for 16 h. Water (40 mL) was added and the mixture was extracted with EtOAc, dried and concentrated in vacuo. The crude product was then purified by column chromatography using 20% ethyl acetate in hexane to give the title compound as a white solid (171.0 mg). Yield: 75%.

### Step 3

To a mixture of methyl 4-((4-chloro-N-(1-phenylpropyl)phenylsulfonamido) methyl)benzoate (118.0 mg, 0.258 mmol) and KOH in a 10-mL flask, MeOH (3 mL) was added and the mixture was stirred at 45° C. for 3 h and cooled to room temperature. All solvent was removed and 5 mL of water was added. The mixture was extracted with EtOAc (3×6 mL) to remove remaining starting material. The aqueous phase was then acidified to pH 2 using 2N HCl solution and then was extracted with EtOAc. The combined organic extracts were dried over Na₂SO₄ and the organic layer was concentrated in vacuo to give a white solid (70 mg, 61%). Elemental Analysis C₂₃H₂₂ClNO₄S: Calcd: C, 62.23, H, 4.99, N, 3.16. Found: C, 62.20, H, 5.02, N, 3.19. Mp 154-156° C.

### Example 28

### Methyl 4-((4-fluoro-N-(2-methyl-1-phenylpropyl)phenylsulfonamido) methyl)benzoate

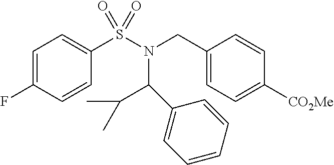 [SMILES: COC(=O)c1ccc(CN(C(c2ccccc2)C(C)C)S(=O)(=O)c2ccc(F)cc2)cc1]

### Step 1

### Methyl 4-((4-fluorophenylsulfonamido)methyl)benzoate

To a mixture of methyl 4-(aminomethyl)benzoate hydrochloride and Et₃N in dichloromethane (DCM) (30 mL) was added 4-fluorobenzenesulfonyl chloride in 20 mL of DCM over 10 minutes using a syringe. After stirring for 1 h, water (100 mL) was added and then extracted with DCM. The combined organic extracts were dried over Na₂SO₄ and concentrated in vacuo. Diethyl ether (100 mL) was then added to the residue and the mixture was stirred at 40° C. for 10 minutes. A white solid precipitated that was filtered and dried to yield the desired product, methyl 4-((4-fluorophenylsulfonamido)methyl)benzoate (5 g, 85%).

### Step 2

To a solution of methyl 4-((4-fluorophenylsulfonamido)methyl)benzoate (162.0 mg, 0.5 mmol, 1.0 eq), 2-methyl-1-phenylpropan-1-ol (152 mg, 1.0 mmol, 2.0 eq) and Ph₃P (292 mg, 1.1 mmol, 2.2 eq) in 5 mL of THF, diisopropyl azodicarboxylate (DIAD) (228 μA, 1.1 mmol, 2.2 eq) was added dropwise. The light yellow mixture was stirred at room temperature for 16 h. Water (20 mL) was added and the mixture was extracted with EtOAc, dried and concentrated in vacuo. The crude product was then purified by column chromatography using 20% ethyl acetate in hexane to give the title compound (125 mg, 55%). ¹H NMR (500 MHz, CDCl₃) δ 7.81 (m, 2H), δ 7.60 (m, 2H), δ 7.22 (m, 4H), δ 7.10 (m, 3H), δ 7.0 (m, 2H), δ 4.61 (d, J=8 Hz, 1H), δ 4.51 (d, J=8 Hz, 1H), δ 4.21 (d, J=11 Hz, 1H), δ 3.92 (s, 3H), δ 2.18 (m, 1H), δ 1.0 (d, J=8 Hz, 3H), δ 0.64 (d, J=8 Hz, 3H),

### Example 29

### Methyl 4-((4-fluoro-N-(1-phenylpropyl)phenylsulfonamido)methyl)benzoate

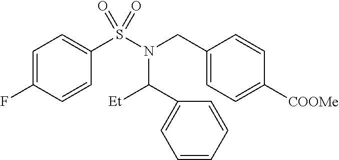 [SMILES: CCC(c1ccccc1)N(Cc1ccc(C(=O)OC)cc1)S(=O)(=O)c1ccc(F)cc1]

### Step 1

### Methyl 4-((4-fluorophenylsulfonamido)methyl)benzoate

To a mixture of methyl 4-(aminomethyl)benzoate hydrochloride and Et₃N in dichloromethane (DCM) (30 mL) was added 4-fluorobenzenesulfonyl chloride in 20 mL of DCM over 10 minutes using a syringe. After stirring for 1 h, 100 mL of water was added and then extracted with DCM. The combined organic extracts were dried over Na₂SO₄ and concentrated in vacuo. Diethyl ether (100 mL) was then added to the residue and the mixture was stirred at 40° C. for 10 minutes. A white solid precipitated that was filtered and dried to yield the desired product, methyl 4-((4-fluorophenylsulfonamido)methyl)benzoate (5 g, 85%).

### Step 2

To a solution of methyl 4-((4-fluorophenylsulfonamido)methyl)benzoate (162 mg, 0.5 mmol, 1.0 eq), 1-phenylpropan-1-ol (137 mg, 1.0 mmol, 2.0 eq) and Ph₃P (292 mg, 1.1 mmol, 2.2 eq) in 5 mL of THF, diisopropyl azodicarboxylate (DIAD) (228 μL, 1.1 mmol, 2.2 eq) was added dropwise. The light yellow mixture was stirred at room temperature for 16 h. Water (20 mL) was added and the mixture was extracted with EtOAc, dried and concentrated in vacuo. The crude product was purified by column chromatography using 20% ethyl acetate in hexane to give the title compound as colorless oil, (55 mg, 25%). Elemental Analysis (C₂₄H₂₄FNO₄S) Calcd: C, 65.29, H, 5.48, N, 3.17. Found: C, 65.56, H, 5.54, N, 3.45.

### Example 30

Methyl 4-((4-chloro-N-(1-(4-fluorophenyl)propyl)phenylsulfonamido) methyl)benzoate

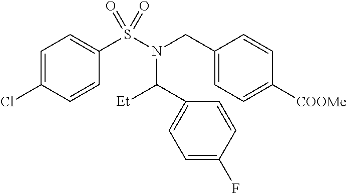 [SMILES: CCC(c1ccc(F)cc1)N(Cc1ccc(C(=O)OC)cc1)S(=O)(=O)c1ccc(Cl)cc1]

### Step 1

### Methyl 4-((4-chlorophenylsulfonamido)methyl)benzoate

To a mixture of methyl 4-(aminomethyl)benzoate hydrochloride (4.16 g, 20 mmol, 1.0 eq) and Et₃N (7 mL, 50 mmol, 2.5 eq) in a 100 mL-round-bottomed flask in dichloromethane (50 mL) was added 4-chlorobenzenesulfonyl chloride (4.35 g 20 mmol, dissolved in 20 mL dichloromethane) over 10 minutes using a syringe at room temperature. After stirring for 1 h, 100 mL of water was added and the aqueous phase was extracted with dichloromethane. The combined organic extracts were dried over Na₂SO₄ and concentrated in vacuo. Diethyl ether (100 mL) was added, the mixture was stirred at 40° C. for 10 minutes and then filtered to give methyl 4-((4-chlorophenylsulfonamido)methyl)benzoate, a white solid (5.77 g, 85%).

### Step 2

To a solution of methyl 4-((4-chlorophenylsulfonamido)methyl)benzoate (166.5 mg, 0.5 mmol, 1.0 eq), 1-(4-fluorophenyl)propan-1-ol (170 mg, 1.1 mmol, 2.0 eq) and Ph₃P (292 mg, 1.1 mmol, 2.2 eq) in 5 mL of THF, diisopropyl azodicarboxylate (DIAD) (228 μA, 1.1 mmol, 2.2 eq) was added dropwise. The light yellow mixture was stirred at room temperature for 16 h. Water (20 mL) was added and the mixture was extracted with EtOAc, dried and concentrated in vacuo. The crude product was purified by column chromatography using 20% ethyl acetate in hexane to give the title compound as a white solid (24 mg, 10%). Elemental Analysis (C₂₄H₂₃ClFNO₄S) Calcd: C, 60.56, H, 4.87, N, 2.94. Found: C, 60.68, H, 4.81, N, 3.05. Mp 95-97° C.

### Example 31

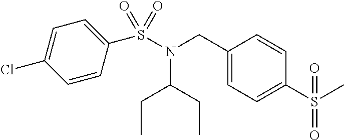 [SMILES: CCC(CC)N(Cc1ccc(S(C)(=O)=O)cc1)S(=O)(=O)c1ccc(Cl)cc1]

### 4-Chloro-N-(4-(methylsulfonyl)benzyl)-N-(pentan-3-yl)benzenesulfonamide

### Step 1

### 4-Chloro-N-(4-(methylsulfonyl)benzyl)benzenesulfonamide

4-Chloro-N-(4-(methylsulfonyl)benzyl)benzenesulfonamide (2.56 g, 69.9%, a light yellow solid), was prepared from 4-chlorophenyl sulfonyl chloride and methyl 4-methylsulphonylbenzylamine hydrochloride according to the general method illustrated in STEP 1, Scheme 1.

### Step 2

### 4-Chloro-N-(4-(methylsulfonyl)benzyl)-N-(pentan-3-yl)benzenesulfonamide

4-Chloro-N-(4-(methylsulfonyl)benzyl)-N-(pentan-3-yl)benzenesulfonamide (191 mg, 88.8%) was prepared from 5-chloro-N-(2,2-dimethyl-1,3-dioxan-5-yl)thiophene-2-sulfonamide and 4-(methylthio)benzyl alcohol according to Method 2 of STEP 2, Scheme 1. Mp 166-168° C. MS (m/z) 430.1 (M⁺+1). Elemental Analysis (C₁₉H₂₄ClNO₄S₂) Calcd: C, 53.07, H, 5.63, N, 3.26. Found: C, 53.26, H, 5.43, N, 3.27.

### Example 32

Methyl 4-((4-fluoro-N-(pentan-3-yl)phenylsulfonamido)methyl)benzoate

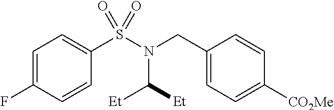

### Step 1

### Methyl 4-((4-fluorophenylsulfonamido) methyl)benzoate

To a mixture of methyl 4-(aminomethyl)benzoate hydrochloride and Et₃N in dichloromethane (DCM) (30 mL), 4-fluorobenzenesulfonyl chloride in 20 mL of DCM was added via a syringe over a 10-min period. After stirring the mixture for 1 h, water (100 mL) was added and the mixture was extracted with DCM. The combined organic extracts were dried over Na₂SO₄ and concentrated in vacuo. Diethyl ether (100 mL) was then added to the residue and the mixture was stirred at 40° C. for 10 minutes. The white precipitate was filtered and dried to give methyl 4-((4-fluorophenylsulfonamido) methyl)benzoate (5.5 g, 85%).

### Step 2

To a solution of methyl 4-((4-fluorophenylsulfonamido)methyl)benzoate (162 mg, 05. mmol, 1.0 eq), 3-propanol (110 μL, 1.0 mmol, 2 equiv) and Ph₃P (292 mg, 1.1 mmol, 2.2 eq) in 5 mL of THF, diisopropyl azodicarboxylate (DIAD) (228 μL, 1.1 mmol, 2.2 eq) was added dropwise. The light yellow mixture was stirred at room temperature for 16 h. Water (40 mL) was added and the mixture was extracted with EtOAc, dried and concentrated in vacuo. The crude product was purified by column chromatography using 20% ethyl acetate/hexane to give the title compound as a white solid, (278 mg, 71%). Mp 78-80° C. Elemental Analysis (C₂₀H₂₄FNO₄S) Calcd: C, 61.05, H, 6.15, N, 3.56. Found: C, 60.80, H, 6.43, N, 3.79.

### Example 33

### 4-Chloro-N-(4-cyanobenzyl)-N-(pentan-3-yl)benzenesulfonamide

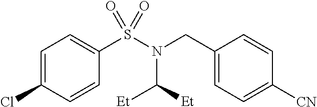

### Step 1

### 4-Chloro-N-(pentan-3-yl)benzenesulfonamide

To a mixture of 4-chlorobenzenesulfonyl chloride (2.61 g, 12 mmol, 1.2 eq) and pyridine (2.43 mL, 30 mmol, 3.0 eq) in a 100 mL-round-bottomed flask in DCM (60 mL), 3-aminopentane (0.89 g, 10 mmol, 1.0 eq, dissolved in 20 mL dichloromethane) was added at room temperature over 10 minutes via a syringe. After stirring 16 h, water (100 mL) was added and the two phases were separated. The aqueous phase was extracted with dichloromethane and the combined organic extracts were dried over Na₂SO₄ and concentrated in vacuo. The crude product was purified by column chromatography to give 4-chloro-N-(pentan-3-yl)benzenesulfonamide, a white solid (2.11 g, 81%).

### Step 2

### 4-Chloro-N-(4-cyanobenzyl)-N-(pentan-3-yl)benzenesulfonamide

To a stirred solution of 4-chloro-N-(pentan-3-yl)benzenesulfonamide (525 mg, 2.0 mmol, 1 eq) and 4-(bromomethyl)benzonitrile (594 mg, 3 mmol, 1.5 eq) in 8 mL of DMF was added K₂CO₃ (830.0 mg, 6.0 mmol, 3.0 equiv) at room temperature. After stirring 16 h, the reaction mixture was quenched with 5 mL of water and extracted with ethyl acetate (2×20 mL). The combined organic extracts were washed with a saturated aqueous Na₂CO₃ solution and brine, and then dried over Na₂SO₄. The mixture was concentrated in vacuo to give crude product. Purification by column chromatography using 20% ethyl acetate in hexane yielded 4-chloro-N-(4-cyanobenzyl)-N-(pentan-3-yl)benzenesulfonamide, a white solid (565 mg, 75%). Mp 123-125° C. Elemental Analysis (C₁₉H₂₁ClN₂O₂S) Calcd: C, 60.55, H, 5.62, N, 7.43. Found: C, 60.64, H, 5.90, N, 7.45.

### Example 34

Methyl 6-((4-fluoro-N-(pentan-3-yl)phenylsulfonamido)methyl)nicotinate

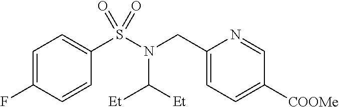 [SMILES: CCC(CC)N(Cc1ccc(C(=O)OC)cn1)S(=O)(=O)c1ccc(F)cc1]

### Step 1

### 4-Fluoro-N-(pentan-3-yl)benzenesulfonamide

To a mixture of 4-fluorobenzenesulfonyl chloride (3.97 g, 20 mmol, 1.0 eq) and pyridine (3.24 mL, 40 mmol, 2.0 eq) in a 200 mL-round-bottomed flask in dichlormethane (100 mL) was added 3-aminopentane (2.13 g, 24 mmol, 1.2 eq, dissolved in 30 mL dichloromethane) at room temperature over 10 minutes using a syringe. After stirring 16 h, water (100 mL) was added and the two phases were separated. The aqueous phase was extracted with dichloromethane. The combined organic extracts were dried over Na₂SO₄, concentrated in vacuo, and the crude product was purified by column chromatography to give 4-fluoro-N-(pentan-3-yl)benzenesulfonamide (3.92 g, 80%), a light yellow oil.

### Step 2

To a stirred solution of 4-fluoro-N-(pentan-3-yl)benzenesulfonamide (247 mg, 1.0 mmol, 1.0 eq) and methyl 6-(bromomethyl)nicotinate (280 mg, 1.2 mmol, 1.2 eq) in 6 mL of DMF was added K₂CO₃ (553 mg, 4 mmol, 4.0 eq) at room temperature. After stirring 16 h, the reaction mixture was quenched with 10 mL of water and then extracted with ethyl acetate (2×20 mL). The combined organic extracts were washed with saturated aqueous Na₂CO₃ solution, brine, and then dried over Na₂SO₄. The organic layers were concentrated in vacuo and then purified by column chromatography (20% EtOAc/hexanes) to give the title compound, a white solid (284 mg, 72%). Mp 117-119° C. Elemental Analysis (C₁₉H₂₃FN₂O₄S) Calcd: C, 57.85, H, 5.88, N, 7.10. Found: C, 57.96, H, 5.73, N, 7.08.

### Example 35

### Methyl 4-((4-methyl-N-(pentan-3-yl)phenylsulfonamido)methyl)benzoate

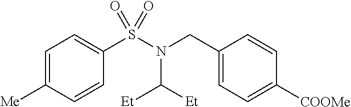 [SMILES: CCC(CC)N(Cc1ccc(C(=O)OC)cc1)S(=O)(=O)c1ccc(C)cc1]

### Step 1

### Methyl 4-((pentan-3-ylamino)methyl)benzoate

Methyl 4-formylbenzoate (6.7 g, 40 mmol, 1.0 eq) was dissolved in 40 mL of methanol at room temperature. 3-Aminopentane (7.10 g, 80 mmol, 2.0 eq) was added and the mixture was stirred at room temperature for 2 h. NaBH₄ (908 mg, 24 mmol, 0.6 eq) was then added in several portions. After stirring for 30 minutes, the solvent was concentrated in vacuo and 100 mL of water was then added. The mixture was then extracted with ethyl acetate and the combined organic extracts were dried over Na₂SO₄, filtered, and concentrated in vacuo to give methyl 4-((pentan-3-ylamino)methyl)benzoate as an oil (9 g, 96%).

### Step 2

Methyl 4-((pentan-3-ylamino)methyl)benzoate (240 mg, 1 mmol, 1.0 eq) and Et₃N (0.22 mL, 1.5 mmol, 1.5 eq) were dissolved in dichloromethane (10 mL) at 0° C. 4-Methylbenzene-1-sulfonyl chloride (388 mg, 2.02 mmol, 2.02 eq) was then added dropwise and the mixture was stirred at room temperature for 16 h. The solvent was evaporated and 10 mL of water and 10 mL of brine were added. The mixture was then extracted with EtOAc and the organic layers were concentrated in vacuo. The crude product was purified by column chromatography using 20% ethyl acetate in hexane to give the title compound as a white solid (327 mg, 84%). Mp 106-108° C. Elemental Analysis (C₂₁H₂₇NO₄S) Calcd: C, 64.75, H, 6.99, N, 3.60. Found: C, 65.92, H, 6.93, N, 3.57.

### Example 36

### N-(4-Cyano-2-fluorobenzyl)-4-fluoro-N-(pentan-3-yl)benzenesulfonamide

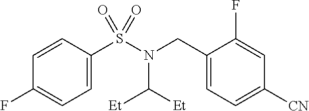 [SMILES: CCC(CC)N(Cc1ccc(C#N)cc1F)S(=O)(=O)c1ccc(F)cc1]

### Step 1

### 4-Fluoro-N-(pentan-3-yl)benzenesulfonamide

To a mixture of 4-fluorobenzenesulfonyl chloride (3.97 g, 20 mmol, 1.0 eq) and pyridine (3.24 mL, 40 mmol, 2.0 eq) in a 200 mL-round-bottomed flask in DCM (100 mL), 3-aminopentane (2.13 g, 24 mmol, 1.2 eq, dissolved in 30 mL dichloromethane) was added at room temperature over 10 minutes using a syringe. After stirring for 16 h, water (100 mL) was added and the two phases were separated. The aqueous phase was extracted with dichloromethane and the combined organic extracts were dried over Na₂SO₄, concentrated in vacuo, and the crude product was then purified by column chromatography using 40% ethyl acetate in hexane to give the title compound, 4-fluoro-N-(pentan-3-yl)benzenesulfonamide, a light yellow oil (3.92 g, 80%).

### Step 2

To a stirred solution of 4-fluoro-N-(pentan-3-yl)benzenesulfonamide (150.0 mg, 0.6 mmol, 1.0 eq) and 4-(bromomethyl)-3-fluorobenzonitrile (154.1 mg, 0.72 mmol, 1.2 eq) in 4 mL of DMF was added K₂CO₃ at room temperature. After stirring for 16 h, the reaction was quenched with 5 mL of water and then extracted with ethyl acetate (2×20 mL). The combined organic extracts were washed with saturated aqueous Na₂CO₃ solution, brine, dried with Na₂SO₄, and then concentrated in vacuo. Purification of the crude product by column chromatography using 20% ethyl acetate in hexane gave the desired product as a white solid (159 mg, 70%). Mp 102-104° C. Elemental Analysis (C₁₉H₂₀F₂N₂O₂S) Calcd: C, 60.30, H, 5.33, N, 7.40. Found: C, 60.09, H, 5.49, N, 7.30.

### Example 37

### 6-((4-Fluoro-N-(pentan-3-yl)phenylsulfonamido)methyl)nicotinic acid

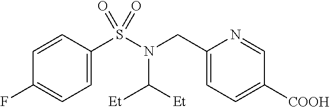 [SMILES: CCC(CC)N(Cc1ccc(C(=O)O)cn1)S(=O)(=O)c1ccc(F)cc1]

### Step 1

### 6-((4-Fluoro-N-(pentan-3-yl)phenylsulfonamido)methyl nicotinate

To a stirred solution of 4-fluoro-N-(pentan-3-yl)benzenesulfonamide (300 mg, 1.2 mmol, 1.0 eq) and methyl 6-(bromomethyl)nicotinate (338 mg, 1.44 mmol, 1.2 eq) in 8 mL of dimethyl formamide at room temperature was added K₂CO₃ (665 mg, 4.8 mmol, 4.0 eq). After stirring at room temperature for 16 h, the reaction mixture was quenched with 5 mL of water and then extracted with ethyl acetate (2×20 mL). The combined organic extracts were washed with saturated aqueous Na₂CO₃ solution and brine, dried over Na₂SO₄, and then solvent was evaporated. The crude product was purified by column chromatography using 20% ethyl acetate in hexane to give methyl 6-((4-fluoro-N-(pentan-3-yl)phenylsulfonamido)methyl nicotinate, a white solid (234 mg, 49.5%).

### Step 2

Methyl 6-((4-fluoro-N-(pentan-3-yl)phenylsulfonamido)methyl)nicotinate (135 mg, 0.34 mmol, 1.0 eq) was suspended in 2 mL of methanol and KOH (45 mg, 0.68 mol, 2.0 eq) was added. The mixture was stirred at 40° C. for 2 h, cooled to room temperature and then 5 mL of water was added. The mixture was acidified to pH 2 using 4N HCl solution. The mixture was extracted with EtOAc and the organic layers were then concentrated in vacuo to give the title compound as a yellow solid (62 mg, 48%). Mp 145-147° C. Elemental Analysis (C₁₈H₂₁FN₂O₄S) Calcd: C, 56.83, H, 5.56, N, 7.36. Found: C, 56.65, H, 5.31, N, 7.16.

### Example 38

### 4-((4-Chloro-N-(pentan-3-yl)phenylsulfonamido)methyl)benzoic acid

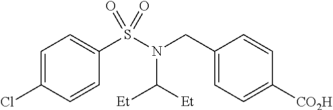 [SMILES: CCC(CC)N(Cc1ccc(C(=O)O)cc1)S(=O)(=O)c1ccc(Cl)cc1]

### Step 1

### 4-Chloro-N-(pentan-3-yl)benzenesulfonamide

To a mixture of 4-chlorobenzenesulfonyl chloride (2.61 g, 12 mmol, 1.2 eq) and pyridine (2.43 mL, 30 mmol, 3.0 eq) in a 100 mL-round-bottomed flask in DCM (60 mL) was added 3-aminopentane (0.89 g, 10 mmol, 1.0 eq, dissolved in 20 mL dichloromethane) over 10 minutes using a syringe at room temperature. After stirring for 16 h, water (100 mL) was added and the aqueous phase was extracted with dichloromethane. The combined organic extracts were dried over Na₂SO₄, concentrated in vacuo to give a crude residue which was purified by column chromatography using 30% ethyl acetate in hexane to give 4-chloro-N-(pentan-3-yl)benzenesulfonamide, a white solid (2.11 g, 81%).

### Step 2

### 4-Chloro-N-(4-cyanobenzyl)-N-(pentan-3-yl)benzenesulfonamide

To a stirred solution of 4-chloro-N-(pentan-3-yl)benzenesulfonamide (525 mg, 2 mmol, 1.0 eq) and 4-(bromomethyl)benzonitrile (594 mg, 3 mmol, 1.5 eq) in 8 mL of DMF was added K₂CO₃ (830 mg, 6 mmol, 3.0 eq) at room temperature. After stirring for 16 h, the reaction mixture was quenched with 5 mL of water and then extracted with ethyl acetate (2×20 mL). The combined extracts were washed with saturated aqueous Na₂CO₃ solution and brine, dried over Na₂SO₄, and concentrated in vacuo to give crude product. Purification by column chromatography using 20% ethyl acetate in hexane gave 4-chloro-N-(4-cyanobenzyl)-N-(pentan-3-yl)benzenesulfonamide, a white solid (565 mg, 75%). Elemental Analysis (C₁₉H₂₁ClN₂O₂S) Calcd: C, 60.55, H, 5.62, N, 7.43. Found: C, 60.64, H, 5.90, N, 7.45. Mp 123-125° C.

### Step 3

N-(2-cyanobenzyl)-4-fluoro-N-(pentan-3-yl)benzenesulfonamide (190 mg, 0.5 mmol) was suspended in 4 mL ethanol and 0.4 mL of 25 N NaOH solution (1 g NaOH+1 mL H₂O) was added. The mixture was refluxed at 87° C. for 20 h, cooled to room temperature and the solvent was evaporated. Water (20 mL) was then added and the mixture was adjusted to pH 2 using 4N HCl and then extracted with EtOAc. The combined extracts were dried over Na₂SO₄ and the crude product was passed through a pad of silica gel to give a waxy solid (49 mg, 25%). ¹H NMR (500 MHz, CDCl₃) δ 8.04 (d, J=7 Hz, 2H), δ 7.72 (d, J=6 Hz, 2H), δ 7.51 (d, J=6.5 Hz, 2H), δ 7.45 (d, J=6 Hz, 2H), δ 4.37 (s, 2H), δ 3.56 (m, 1H), δ 1.37 (m, 2H), δ 1.21 (m, 2H), δ 0.71 (t, J=6 Hz, 6H).

### Example 39

### N-(4-Cyano-2-fluorobenzyl)-4-fluoro-N-(pentan-3-yl)benzenesulfonamide

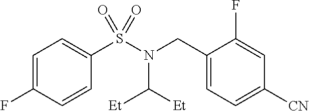 [SMILES: CCC(CC)N(Cc1ccc(C#N)cc1F)S(=O)(=O)c1ccc(F)cc1]

### Step 1

### Methyl 4-((4-chlorophenylsulfonamido) methyl)benzoate

To a mixture of methyl 4-(aminomethyl)benzoate hydrochloride and Et₃N in dichloromethane (DCM) (30 mL) was added 4-chlorobenzenesulfonyl chloride in 20 mL of DCM over 10 minutes using a syringe. After stirring for 1 h, 100 mL of water was added and the mixture was extracted with DCM. The combined organic extracts were dried over Na₂SO₄ and concentrated in vacuo. Diethyl ether (100 mL) was then added and the mixture was stirred at 40° C. for 10 minutes. The white precipitate was filtered and dried to give methyl 4-((4-chlorophenylsulfonamido) methyl)benzoate (7.5 g, 90%).

### Step 2

To a stirred solution of 4-chloro-N-(pentan-3-yl)benzenesulfonamide (300 mg, 1.15 mmol) and 4-(bromomethyl)-3-fluorobenzonitrile (294 mg, 1.38 mmol) in 4 mL of DMF was added K₂CO₃ at room temperature and the mixture was stirred for 16 h. The solvent was evaporated and 10 mL of water was added. This mixture was extracted with EtOAc to give a crude product that was purified by column chromatography using 20% ethyl acetate in hexane. The title compound was isolated as a white solid (160 mg, 35%). Elemental Analysis (C₁₉H₂₀F₂N₂O₂S) Calcd: C, 60.30, H, 5.33, N, 7.40. Found: C, 57.82, H, 5.03, N, 7.16. Mp 102-104° C.

### Example 40

### Methyl 4-((4-chloro-N-(pentan-3-yl)phenylsulfonamido)methyl)benzoate

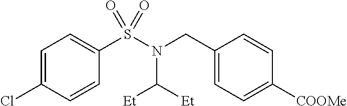 [SMILES: CCC(CC)N(Cc1ccc(C(=O)OC)cc1)S(=O)(=O)c1ccc(Cl)cc1]

### Step 1

### Methyl 4-((4-chlorophenylsulfonamido) methyl)benzoate

To a mixture of methyl 4-(aminomethyl)benzoate hydrochloride and Et₃N in dichloromethane (DCM) (30 mL) was added 4-chlorobenzenesulfonyl chloride in 20 mL of DCM over 10 minutes using a syringe. After stirring for 1 h, 100 mL of water was added and the mixture was extracted with DCM. The combined organic extracts were dried over Na₂SO₄ and concentrated in vacuo. Diethyl ether (100 mL) was then added and the mixture was stirred at 40° C. for 10 minutes. The white precipitate was filtered and dried to give methyl 4-((4-chlorophenylsulfonamido) methyl)benzoate (7.5 g, 90%).

### Step 2

To a solution of methyl 4-((4-chlorophenylsulfonamido)methyl)benzoate (152 mg, 0.44 mmol, 1.0 eq), pentan-3-ol (97 μA, 0.88 mmol, 2.0 eq) and Ph₃P (255 mg, 0.97 mmol, 2.2 eq) in 5 mL of THF, diisopropyl azodicarboxylate (DIAD) (202 μA, 0.97 mmol, 2.2 eq) was added dropwise. The light yellow mixture was stirred at room temperature for 16 h. Water (20 mL) was added and the mixture was extracted with EtOAc, dried and concentrated in vacuo. The crude product was purified by column chromatography using 20% ethyl acetate in hexane to give the title compound as a white solid (128 mg. 71%). Elemental Analysis (C₂₀H₂₄ClNO₄S) Calcd: C, 58.60, H, 5.90, N, 3.42. Found: C, 58.88, H, 4.90, N, 3.47. Mp 95-97° C.

### Example 41

### Methyl 4-0N-(1,3-difluoropropan-2-yl)-4-fluorophenylsulfonamido)methyl)benzoate

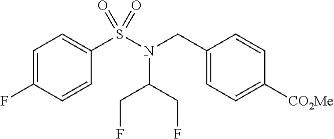 [SMILES: COC(=O)c1ccc(CN(C(CF)CF)S(=O)(=O)c2ccc(F)cc2)cc1]

### Step 1

### Methyl 4-((4-fluorophenylsulfonamido)methyl)benzoate

To a mixture of methyl 4-(aminomethyl)benzoate hydrochloride and Et₃N in dichloromethane (DCM) (30 mL) was added 4-fluorobenzenesulfonyl chloride in 20 mL of DCM over 10 minutes using a syringe. After stirring for 1 h, 100 mL of water was added to the mixture and then extracted with DCM. The combined organic extracts were dried over Na₂SO₄ and concentrated in vacuo. Diethyl ether (100 mL) was then added to the residue and the mixture was stirred at 40° C. for 10 minutes. A white solid precipitated that was filtered and dried to yield the desired product, methyl 4-((4-fluorophenylsulfonamido)methyl)benzoate (5 g, 85%).

### Step 2

To a solution of methyl 4-((4-fluorophenylsulfonamido)methyl)benzoate (162 mg, 0.5 mmol, 1.0 eq), 1,3-difluoro-2-propanol (77.4 μL, 1 mmol, 2.0 eq) and Ph₃P (292 mg, 1.1 mmol, 2.2 eq) in 5 mL of THF, diisopropyl azodicarboxylate (DIAD) (228 μL, 1.1 mmol, 2.2 eq) was added dropwise. The light yellow mixture was stirred at room temperature for 16 h. Water (40 mL) was added and the mixture was extracted with EtOAc, dried and concentrated in vacuo. The crude product was purified by column chromatography using 20% ethyl acetate in hexane to give the title compound as a white solid, (150 mg, 75%). Elemental Analysis (C₁₈H₁₈F₃NO₄S) Calcd: C, 53.86, H, 4.52, N, 3.49. Found: C, 53.77, H, 4.29, N, 3.75. Mp 103-105° C.

### Example 42

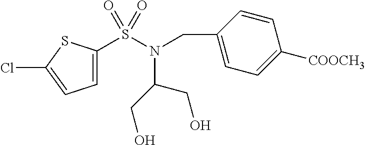 [SMILES: COC(=O)c1ccc(CN(C(CO)CO)S(=O)(=O)c2ccc(Cl)s2)cc1]

Methyl 4-((5-chloro-N-(1,3-dihydroxypropan-2-yl)thiophene-2-sulfonamido) methyl)benzoate

### Step 1

### 5-Chloro-N-(1,3-dihydroxypropan-2-yl)thiophene-2-sulfonamide

5-Chloro-N-(1,3-dihydroxypropan-2-yl)thiophene-2-sulfonamide (3.39 g, 54.2%) was prepared from 5-chlorothiophene-2-sulfonyl chloride and 2-amino-1,3-propanediol according to the general method described for STEP 1, Scheme 1. Mp 90-92° C. MS (m/z) 270.9 (M⁺).

### Step 2

### 5-Chloro-N-(2,2-dimethyl-1,3-dioxan-5-yl)thiophene-2-sulfonamide

To a solution of 5-chloro-N-(1,3-dihydroxypropan-2-yl)thiophene-2-sulfonamide (3 g, 11.03 mmol) in 50 mL of THF was added dimethoxyacetone (5.74 g, 55.2 mmol, 5 eq) and p-toluene sulfonic acid monohydrate (210 mg, 1.1 mmol, 0.1 eq). The reaction mixture was stirred at room temperature for less than 1.5 h. The reaction mixture was treated with aqueous NaHCO₃ solution immediately after the starting material was consumed. This mixture was then stirred for 10 minutes and Na₂CO₃ solution was added to adjust the mixture to pH 11. THF was removed and the residue was partitioned between ethyl acetate and water. The organic layers were separated and washed with water and brine and dried over Na₂SO₄. Subsequent filtration and concentration in vacuo of the organic layers provided crude product that was recrystallized in hot ethyl acetate to give 5-chloro-N-(2,2-dimethyl-1,3-dioxan-5-yl)thiophene-2-sulfonamide (2.28 g, 66.4%).

### Step 3

### Methyl 4-((5-chloro-N-(2,2-dimethyl-1,3-dioxan-5-yl)thiophene-2-sulfonamido)methyl)benzoate

Methyl 4-((5-chloro-N-(2,2-dimethyl-1,3-dioxan-5-yl)thiophene-2-sulfonamido)methyl)benzoate (220 mg, 82.1%) was prepared from 5-chloro-N-(2,2-dimethyl-1,3-dioxan-5-yl)thiophene-2-sulfonamide and methyl 4-(hydroxymethyl)benzoate according to the general method described for Method 2 of STEP 2, Scheme 1.

### Step 4

### Methyl 4-((5-chloro-N-(1,3-dihydroxypropan-2-yl)thiophene-2-sulfonamido)methyl)benzoate

To a solution of methyl 4-((5-chloro-N-(2,2-dimethyl-1,3-dioxan-5-yl)thiophene-2-sulfonamido)methyl)benzoate (156 mg, 0.34 mmol) in 5 mL of THF was added 0.5 mL of methanol and p-toluene sulfonic acid monohydrate (67.7 mg, 0.35 mmol, 1.1 eq). The reaction mixture was stirred at room temperature for 3 h. Na₂CO₃ aqueous solution was then added to the mixture to adjust to pH 11. THF was removed in vacuo and the residue was partitioned between ethyl acetate and water. The organic layers were separated and washed with water, brine and dried over Na₂SO₄. Subsequent filtration of the organic layers and concentration in vacuo provided crude product that was purified using flash chromatography (silica gel column, 10-70% ethyl acetate in hexane) to yield methyl 4-((5-chloro-N-(1,3-dihydroxypropan-2-yl)thiophene-2-sulfonamido)methyl)benzoate (110 mg, 77.2%). Mp 134-135° C. MS (m/z) 420.0 (M⁺+1). Elemental Analysis (C₁₆H₁₈ClNO₆S₂) Calcd: C, 45.77, H, 4.32, N, 3.34. Found: C, 46.05, H, 4.06, N, 3.20.

### Example 43

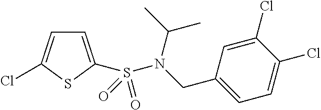 [SMILES: CC(C)N(Cc1ccc(Cl)c(Cl)c1)S(=O)(=O)c1ccc(Cl)s1]

### 5-Chloro-N-(3,4-dichlorobenzyl)-N-isopropylthiophene-2-sulfonamide

To a solution of 5-chlorothiophene-2-sulfonyl chloride (108.5 mg, 0.5 mmol) in 4 mL of CH₃CN, N-(3,4-dichlorobenzyl)propan-2-amine (108.4 mg, 0.55 mmol, 1.1 eq) and triethylamine (0.68 mmol, 1.25 eq) were added. The reaction mixture was stirred at room temperature for 16 h and then quenched with water. CH₃CN was concentrated in vacuo and the crude residue was extracted with ethyl acetate. The organic layers were separated and washed with 1N HCl, aqueous Na₂CO₃, water and brine, and then dried over Na₂SO₄. Subsequent filtration and concentration in vacuo provided a crude product that was purified by flash chromatography using 10-25% ethyl acetate in hexane to yield the title compound (78 mg, 39.1%). Mp 66-67° C. Elemental Analysis (C₁₄H₁₄C₁₃NO₂S₂) Calcd: C, 42.17, H, 3.54, N, 3.51. Found: C, 42.52, H, 3.44, N, 3.48, MS (m/z) 398.2 (M⁺).

### Example 44

### Methyl 4-((4-chloro-N-isopropylphenylsulfonamido)methyl)benzoate

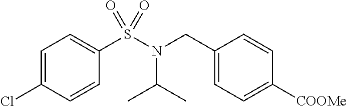 [SMILES: COC(=O)c1ccc(CN(C(C)C)S(=O)(=O)c2ccc(Cl)cc2)cc1]

### Step 1

### Methyl 4-((4-chlorophenylsulfonamido)methyl)benzoate

To a mixture of methyl 4-(aminomethyl)benzoate hydrochloride (4.16 g, 20 mmol, 1.0 eq) and Et₃N (7 mL, 50 mmol, 2.5 eq) in a 100 mL-round-bottomed flask in dichloromethane (50 mL), 4-chlorobenzenesulfonyl chloride (4.35 g 20 mmol, dissolved in 20 mL dichloromethane) was added at room temperature over a 10-min period via a syringe. After stirring for 1 h, water (100 mL) was added and the two phases were separated. The aqueous phase was extracted with dichloromethane and the combined organic extracts were dried over Na₂SO₄ and concentrated in vacuo. Diethyl ether (100 mL) was then added and the mixture was stirred at 40° C. for 10 minutes and filtered to give a white solid (5.77 g, 85%).

### Step 2

### 4-((4-Chloro-N-isopropylphenylsulfonamido)methyl)benzoate

To a solution of methyl 4-((4-chlorophenylsulfonamido)methyl)benzoate (152 mg, 0.44 mmol, 1 eq), 2-propanol (67.4 μL, 0.88 mmol, 2 equiv) and Ph₃P (255 mg, 0.97 mmol, 2.2 eq) in 5 mL of THF, diisopropyl azodicarboxylate (DIAD) (202 μL, 0.97 mmol, 2.2 eq) was added dropwise. The light yellow mixture was stirred at room temperature for 16 h. Water (40 mL) was added and the mixture was extracted with EtOAc. The combined organic extracts were dried over Na₂SO₄, concentrated in vacuo, and then purified by column chromatography (20% EtOAc/hexanes) to give methyl 4-((4-chloro-N-isopropylphenylsulfonamido)methyl)benzoate, a white solid (114.0 mg, 68%). Mp 110-112° C. Elemental Analysis (C₁₈H₂₀ClNO₄S) Calcd: C, 56.61, H, 5.28, N, 3.67. Found: C, 56.90, H, 5.06, N, 3.64.

### Example 45

Methyl 4-((4-fluoro-N-isopropylphenylsulfonamido)methyl)benzoate

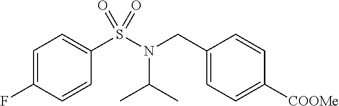 [SMILES: COC(=O)c1ccc(CN(C(C)C)S(=O)(=O)c2ccc(F)cc2)cc1]

### Step 1

Methyl 4-((4-fluorophenylsulfonamido)methyl)benzoate

To a mixture of methyl 4-(aminomethyl)benzoate hydrochloride and Et₃N in dichloromethane (DCM) (30 mL) was added 4-fluorobenzenesulfonyl chloride in 20 mL of DCM over 10 minutes using a syringe. After stirring for 1 h, 100 mL of water was added and then extracted with DCM. The combined organic extracts were dried over Na₂SO₄ and concentrated in vacuo. Diethyl ether (100 mL) was then added to the residue and the mixture was stirred at 40° C. for 10 minutes. A white solid precipitated that was filtered and dried to yield the desired product, methyl 4-((4-fluorophenylsulfonamido)methyl)benzoate (5 g, 85%).

### Step 2

To a solution of methyl 4-((4-fluorophenylsulfonamido)methyl)benzoate (145 mg, 0.44 mmol, 1.0 eq), 2-propanol (67.4 μL, 0.88 mmol, 2.0 eq) and Ph₃P (255 mg, 0.97 mmol, 2.2 eq) in 5 mL of THF, diisopropyl azodicarboxylate (DIAD) (202 μL, 0.97 mmol, 2.2 eq) was added dropwise. The light yellow mixture was stirred at room temperature for 16 h. Water (20 mL) was added and the mixture was extracted with EtOAc, dried and concentrated in vacuo. The crude product was purified by column chromatography using 20% ethyl acetate in hexane to give the title compound as a white solid, (101 mg, 63%). Elemental Analysis (C₁₈H₂₀FNO₄S) Calcd: C, 59.16, H, 5.52, N, 3.83. Found: C, 59.74, H, 4.54, N, 3.86. Mp 118-120° C. MS (m/z) 366.1 (M⁺+H).

### Example 46

### Methyl 4-((5-chloro-N-isopropylthiophene-2-sulfonamido)methyl)benzoate

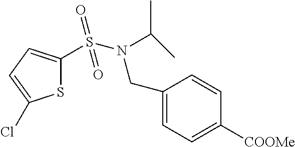 [SMILES: COC(=O)c1ccc(CN(C(C)C)S(=O)(=O)c2ccc(Cl)s2)cc1]

### Step 1

### Methyl 4-((5-chlorothiophene-2-sulfonamido)methyl)benzoate

To a mixture of methyl 4-(aminomethyl)benzoate (304 mg, 1.84 mmol) and triethylamine (466 mg, 4.61 mmol) in dichloromethane (DCM) (5 mL), 5-chlorothiophene-2-sulfonyl chloride (400 mg, 1.84 mmol) in DCM (2 mL) was added. After stirring for 2 h, the solution was diluted with ethyl acetate and washed with water. The organic layer was concentrated in vacuo and the mixture was purified by column chromatography (hexane/ethyl acetate) to give the product as a white solid (400 mg, 63%).

### Step 2

### Methyl 4-((5-chloro-N-isopropylthiophene-2-sulfonamido)methyl)benzoate

To the solution of methyl 4-((5-chlorothiophene-2-sulfonamido)methyl)benzoate (200 mg, 0.58 mmol), propan-2-ol (69.5 mg, 1.16 mmol), and triphenylphosphine (334 mg, 1.27 mmol), diisopropyl azodicarboxylate (257 mg, 1.27 mmol) was added dropwise. After 3 h, the reaction mixture was diluted with ethyl acetate and washed with water. The organic layer was concentrated in vacuo and the crude product was purified by column chromatography (hexane/ethyl acetate) to give the desired product as oil (150 mg, 67%). MS (m/z) 388.3. Elemental Analysis Calcd: C, 49.54, H, 4.68, N, 3.61. Found: C, 50.07, H, 5.02, N, 4.04.

### Example 47

### N-(3,4-Dichlorobenzyl)-N-isopropylbenzofuran-2-sulfonamide

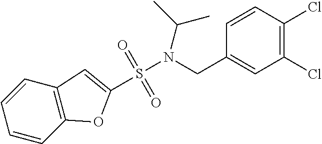 [SMILES: CC(C)N(Cc1ccc(Cl)c(Cl)c1)S(=O)(=O)c1cc2ccccc2o1]

To a solution of benzofuran-2-sulfonyl chloride (108.3 mg, 0.5 mmol) in 4 mL of dichloromethane was added pyridine (5 mmol, 10 eq) and N-(3,4-dichlorobenzyl)propan-2-amine (108.4 mg, 0.55 mmol, 1.1 eq). The reaction mixture was stirred at room temperature for 16 h and then quenched with water. The organic layer was separated and washed with 2 N HCl, water, aqueous Na₂CO₃, and brine and then dried with Na₂SO₄. Subsequent filtration and concentration in vacuo provided a crude product that was purified by flash chromatography using 25% ethyl acetate in hexane to yield the title compound (42 mg, 20.6%). High Resolution Mass Spectrometry (C₁₈H₁₇C₁₂NO₃S) Calcd: 397.03062. Found: 397.03026.

### Example 48

### N-(3,4-Dichlorobenzyl)-N-isopropylbenzo[b]thiophene-2-sulfonamide

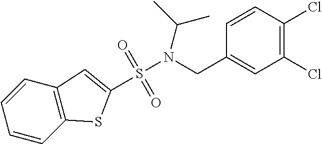 [SMILES: CC(C)N(Cc1ccc(Cl)c(Cl)c1)S(=O)(=O)c1cc2ccccc2s1]

To a solution of benzo[b]thiophene-2-sulfonyl chloride (116.4 mg, 0.5 mmol) in 4 mL of dichloromethane was added pyridine (5 mmol, 10 eq) and N-(3,4-dichlorobenzyl)propan-2-amine (108.4 mg, 0.55 mmol, 1.1 eq). The reaction mixture was stirred at room temperature for 16 h and then quenched with water. The organic layer was separated and washed with 2 N HCl, water, aqueous Na₂CO₃, and brine and then dried with Na₂SO₄. Subsequent filtration and concentration in vacuo provided a crude product that was purified by flash chromatography using 20% ethyl acetate in hexane to yield the title compound as a white solid (72 mg, 34.7%). Mp 133-135° C.; Elemental Analysis (C₁₈H₁₇Cl₂NO₂S₂) Calcd: C, 52.17, H, 4.14, N, 3.38. Found: C, 52.19, H, 4.09, N, 3.34, MS (m/z) 414.4 (M⁺+1).

### Example 49

This Example describes assays performed to evaluate the biological activity of the compounds described herein.

Cell Lines and Cultures.

HeLa S3 cells, the Chinese hamster ovary (CHO) 7[γ]-cell line (co-expressing human PS1, FLAG-Pen-2, and Aph1[alpha]2-HA), and the S—I CHO cell line (co-expressing human PS1, FLAG-Pen-2, Aph1[alpha]2-HA, and NCT-GST) were cultured using reported methods. (See Fraering, P. C, Ye, W., Strub, J. M., Dolios, G., LaVoie, M. J., Ostaszewski, B. L., Van Dorsselaer, A., Wang, R., Selkoe, D. J., and Wolfe, M. S. (2004) Biochemistry 43, 9774-9789; Kimberly, W. T., Esler, W. P., Ye, W., Ostaszewski, B. L., Gao, J., Diehl, T., Selkoe, D. J., and Wolfe, M. S. (2003) Biochemistry 42, 137-144; Fraering, P. C, LaVoie, M. J., Ye, W., Ostaszewski, B. L., Kimberly, W. T., Selkoe, D. J., and Wolfe, M. S. (2004) Biochemistry 43, 323-333.

Purification of γ-Secretase and In Vitro γ-Secretase Assays.

The following procedures can be used to isolate γ-secretase and measure its enzymatic activity. The multistep procedure for the high grade purification of human γ-secretase from the S—I cells uses reported methods (Fraering, P. C, et al. (2004) Biochemistry 43, 9774-9789). In vitro γ-secretase assays using the recombinant APP-based substrate C-100 FLAG and the recombinant Notch-based substrate N-100 FLAG have also been reported(Esler, W. P., Kimberly, W. T., Ostaszewski, B. L., Ye, W., Diehl, T. S., Selkoe, D. J., and Wolfe, M. S. (2002) Proc. Natl. Acad. Sci. U.S.A. 99, 2720-2725, Kimberly, W. T., et al. (2003) Biochemistry 42, 137-144). Basically, the proteolytic reaction mixtures contain C-100 FLAG and N-100 FLAG substrates at a concentration of 1 [μM], purified γ-secretase solubilized in 0.2% CHAPSO/HEPES, pH 7.5, at 10-fold dilution from stock (stock=the M2 anti-FLAG-eluted fraction in the purification protocol from S—I cells (Fraering, P. C, et al. (2004) Biochemistry 43, 9774-9789)), 0.025% phosphatidylethanolamine (PE) and 0.10% phosphatidylcholine (PC). All the reactions are stopped by adding 0.5% SDS, and the samples are assayed for Aβ 40 and Aβ 42 by ELISA (Xia, W., Zhang, J., Ostaszewski, B. L., Kimberly, W. T., Seubert, P., Koo, E. H., Shen, J., and Selkoe, D. J. (1998) Biochemistry 31, 16465-16471). The capture antibodies are 2G3 (to Aβ residues 33-40) for the Aβ 40 species and 21F12 (to Aβ residues 33-42) for the Aβ 42 species.

Western Blotting and Antibodies.

The following assay can be used to determine the extent to which the compounds of interest modulate the cleavage of APP and the Notch receptor. For Western analysis of PS1-NTF, PS1-CTF, Aphl-α2-HA, FLAG-Pen-2, and NCT-GST, the samples are run on 4-20% Tris-glycine polyacrylamide gels, transferred to polyvinylidene difluoride, and can be probed with Aβ14 (for PS1-NTF, 1:2000; a gift of S. Gandy), 13A11 (for PS1-CTF, 5 μg/mL; a gift of Elan Pharmaceuticals), 3F10 (for Aphlα2-HA, 50 ng/mL; Roche Applied Science), anti-FLAG M2 (for FLAG-Pen-2, 1:1000; Sigma), or α-GST antibodies (for NCT-GST, 1:3000; Sigma). Samples from the γ-secretase activity assays (above) are run on 4-20% Tris-glycine gels and can be transferred to polyvinylidene difluoride membranes to detect AICD-FLAG with anti-FLAG M2 antibodies (1:1000, Sigma) and NICD-FLAG with Notch Aβ 1744 antibody (1:1000, Cell Signaling Technology), which is selective for the N terminus of NICD; the same samples are transferred to nitrocellulose membranes to detect Aβ with the anti-Aβ 6E10 antibody. Levels of AICD-FLAG and NICD-FLAG are estimated by densitometry using AlphaEase/Spot Denso (Alpha Innotech Corp.).

Purified γ-Secretase and Binding to ATP-Immobilized Resins.

The following assay can be used to determine the extent to which the compounds of interest bind to ATP. The purified [gamma]-secretase is diluted 10-fold from stock (Fraering, P. C, et al. (2004) Biochemistry 43, 9774-9789) in 50 mM HEPES buffer, pH 7.0, containing 0.2 or 1% CHAPSO, 150 mM NaCl, 5 mM MgCl₂, 5 mM CaCb and can be incubated overnight, in the presence or absence of 50 mM ATP (Sigma), with ATP-agarose (ATP attached to agarose through the ribose hydroxyls, Sigma catalog number A-4793) or ATP-acrylamide (ATP attached to acrylamide through the γ-phosphate; Novagen catalog number 71438-3). Each resin is washed three times with 0.2 or 1% CHAPSO/HEPES buffer, and the bound proteins are collected in 2× Laemmli sample buffer, and can be resolved on 4-20% Tris-glycine gels, and then transferred to polyvinylidene difluoride membranes to detect NCT-GST, PS1-NTF3 Aphl-HA, PS1-CTF, and FLAG-Pen2 as described above.

Photoaffinity Labeling Experiments.

The following assay can be used to determine the extent to which the compounds of interest inhibit the cleavage of APP. 8-Azido-[γ-³²P]ATP (18 Ci/mmol) is purchased from Affinity Labeling Technology (Lexington, Ky.). For the photoaffinity labeling of the purified γ-secretase, the enzyme is diluted 10-fold from stock (Fraering, P. C, et al. (2004) Biochemistry 43, 9774-9789) in 50 mM HEPES buffer, pH 7.0, containing 0.2% CHAPSO, 150 mM NaCl, 5 mM MgCl2, 5 mM CaCl₂, 0.025% PE, and 0.10% PC. The samples are exposed to UV light for 5 min (hand-held UV lamp at 254 nm; UVP model UVGL-25) on ice, and the reaction is quenched with 1 mM dithiothreitol. The proteins are diluted in 0.5% CHAPSO/HEPES buffer and incubated overnight for affinity precipitation with GSH resin as described previously (Fraering, P. C, et al. (2004) Biochemistry 43, 9774-9789, Fraering, P. C, et al. (2004) Biochemistry 43, 323-333). The unbound nucleotides are removed by washing the resin three times and then the washed proteins are resuspended in Laemmli sample buffer. For the photoaffinity labeling of the purified [gamma]-secretase followed by the BN-PAGE analysis, the enzyme is diluted in 0.1% digitonin/TBS, exposed to UV light for 5 min, and directly loaded onto a 5-13.5% BN-polyacrylamide gel. For the photoaffinity labeling of endogenous γ-secretase, HeLa S3 membranes (the equivalent of 3.0×10⁸ cells) are incubated with 22.5 μM 8-azido-[γ-³²P]ATP (10 μCiper reaction), 50 mM HEPES, pH 7.0, 150 mMNaCl, 5 mM MgCl₂, and 5 mMCaCl₂ in a total volume of 60 μL for 10 min at 37° C. The resuspended membranes are exposed to UV light as described above. The unbound nucleotides are removed by washing the membranes three times and then the washed membranes are resuspended for 1 h in 0.5 ml of 1% CHAPSO/HEPES, pH 7.4. The solubilized proteins are diluted 1:2 in HEPES buffer (final CHAPSO concentration=0.5%) and incubated overnight with X81 antibody for immunoprecipitation, as described previously (Fraering, P. C, et al. (2004) Biochemistry 43, 9774-9789, Fraering, P. C, et al. (2004) Biochemistry 43, 323-333). Samples are electrophoresed on 4-20% Tris-glycine gels and autoradiographed (BioMax MS films used with BioMax Transcreen HE (Eastman Kodak Co.)).

ATPase Assays.

The following assay can be used to determine if the compounds of interest compete with ATP. [(X-³²P]ATP (11.9 Ci/mmol) is purchased from Affinity Labeling Technology (Lexington, Ky.). The purified γ-secretase is prepared as described for the photoaffinity labeling experiments; 5 μCi of [(X-³²P]ATP was added; the reactions are incubated at 37° C., and at the indicated time points aliquots are removed and reactions stopped by addition of 10% SDS. A total of 2 μL of each stopped reaction is analyzed by TLC onpolyethyleneimine cellulose plastic sheets (Baker-Flex, Germany) with 0.75 M KH₂PO₄, pH 3.5, as the running buffer to separate ATP from ADP. To identify hydrolysis products, a reaction of [α-³²P]ATP can be incubated in the presence of 0.005 units of canine kidney phosphatase (Sigma). Samples are autoradiographed as described above.

Aβ (1-42) Cellular Assay.

The following assay is used to determine the extent to which the compounds of interest inhibit the cleavage of APP in vivo. AβELISA is a commercial fluorometric kit from Biosource (Invitrogen 89344). Luciferase reporter HEK AP-GL-Tl 6 cells are plated at 50,000 cells/well in 96 well plates in DMEM media containing 10% tetracyclin free BSA, 250 μg/mL zeocin, 200 μg/mL hygromycin, and 54 mL blasticidin. Compounds are added 24 h after plating and APP processing is induced simultaneously by addition of tetracycline. Following a 24 h compound treatment, 50 mL of conditioned cell media is collected, mixed with ELISA diluent buffer containing 2 mM AEBSF and 12 mM o-phenanthroline, and immediately frozen at −80° C. For the ELISA, the samples are brought to room temperature and spun at 5000 rpm for 5 min. Samples (50 mL) are incubated in the ELISA plate with 50 mL detection antibody on a shaker at room temperature for 3 h. Wells are then washed 4 times with wash buffer and 100 mL of secondary antibody are added and incubated at room temperature for 30 min. Wells are again washed 4 times with wash buffer and 100 mL of fluorescent substrate solution are added. After 30 minutes of incubation, fluorescent signals are determine on a Gemini reader at ex 460 nm and em 560 nm. The amount of Aβ levels in each sample is determined from a standard curve generated by known concentrations of Aβ peptide run simultaneously with the samples.

EC₅₀ Determination with Tetracycline.

Cells are trypsinized using trypsin-EDTA (Invitrogen) and harvested by centrifugation at 151 Og. The pellet is then resuspended with DMEM-HZB. The density of cells is determined with a hematocytometer, and cells (500 cellsμL) are transferred at 40 μL/well into 384-well Nunc cell culture plates. Cells are incubated at 37° C. in a CO₂ incubator for 24 h. Serially diluted tetracycline is added to media starting from a 5 μg/mL concentration on a separate plate. For each concentration, 10 wells are used. For negative control, no tetracycline is added to media. On the second day, 10 μL/well of media with/without tetracycline is added. After an additional 48 h of incubation, the plates are brought to room temperature, and 50 μL of luciferase substrate is added. The luminescence is then read using an LJL Analyst (Molecular Device).

IC₅₀ Determination of a γ-Secretase Inhibitor.

The following assay can be used to determine the concentration of a compound of the invention required to achieve 50% inhibition of γ-secretase activity. Serial 3-fold dilutions of compound E, a potent inhibitor of γ-secretase, starting at 3 μM final concentration, are prepared on a separate plate using media with tetracycline, and 10 μL of each is added to 384-well Nunc white plates containing cells (final concentration of tetracycline is 1 μg/mL). Ten replicates are used for each concentration, and the experiment is performed 3 times. The plates are further incubated for 48 h after tetracycline addition. After bringing the temperature down to room temperature, 50 μL of luciferase substrate/well is added and mixed, and luminescence is recorded with an LJL Analyst (Molecular Device).

MTS Assay.

The following assay can be used to indicate the number of viable cells in proliferation and thereby evaluate the toxicity of a candidate compound. The MTS assay used is Promega's Cell Titer 96 Aqueous One Solution Cell Proliferation Assay. It is a colorometric assay that indicates the number of viable cells in proliferation by measuring the amount of MTS reduced to formazan by NADPH or NADH produced by metabolically active cells. After conditioned media is collected for the ELISA, MTS reagent is added to sample at a ratio of 20 mL reagent to 100 mL cell media. Samples are incubated for 1 h at 37° C. in a 5% CO₂ incubator. Then absorbance is recorded at 490 run with a Gemini reader. Cell viability is assessed by determining the percent sample signal to untreated controls. All sample and control signals are adjusted to a background signal determined from cells lysed with 0.9% Triton X.

Notch Cellular Assay.

This assay is used to determine if the compounds of interest inhibit the cleavage of Notch by γ-secretase in cells. A U2OS cell line in which the luciferase expression is adjusted by active Notch is used in this assay Notch expression is adjusted using Tet-on promoter. Luciferase reporter U2OS cells are plated at 1000 cells/well in 96 well plates in DMEM containing 100 μg/mL hygromycin, 15 μg/mL blasticidin and 1 μg/mL Tetracycline. Compounds are added 24 h after plating and the cells are lysed 6 days after adding compounds. Luciferase expression is performed using Steady-Glo Luciferase Assay System (Promega).

### Example 49A

### Acute In Vivo Efficacy Study of Example 1

A preliminary acute efficacy study in 6-month-old female hAPPSL transgenic (Tg) mice (Rockenstein, E., Mallory, M., Mante, M., Sisk, A., and Masliah, E. (2001) J Neurosci Res 66, 573-582) was completed on the compound of Example 1. Mice were treated orally (b.i.d.) with two doses, 50 mg/kg (n=4) and 100 mg/kg (n=4) for 7 consecutive days. No mice died during these AD749 treatments, and no obvious adverse side effects on any organ in either dosing group compared to vehicle controls were observed. Human Aβ38, Aβ40 and Aβ42 levels were determined in the cortex, hippocampus, and cerebral spinal fluid (CSF) by an immunosorbent assay.

Statistically significant reductions of Aβ38 (p<0.05) and Aβ40 (p<0.01) in the hippocampus were observed when the mice were treated with the compound of Example 1.

Chronic In Vivo Efficacy

The compound of Example 1 is administered in a chronic in vivo study. Female hAPPSL at the age of 8-9 months are allocated to 2 different treatment groups: vehicle and the compound of Example 1. Mice are dosed orally (100 mg/kg) twice daily for two months. At the end of the treatment, behavior of animals is evaluated using the Morris Water Maze test system. After the behavioral testing, the mice are sacrificed, and the blood, CSF, and brains will be collected and used for analysis as described herein.

General Procedure for Synthesis of Aryl Sulfonamide Amide Analogs of Example 1

To a mixture of (S)-4-((4-chloro-N-(1-phenylpropyl)phenylsulfonamido)methyl)benzoic acid (1 mmol), a selected amine (1.2 mmol), EDC (1.2 mmol) and HATU (O-(7-azabenzotriazol-1-yl)-N,N,N′,N′-tetramethyluronium hexafluorophosphate (1.2 mmol)) in 2 mL of anhydrous DMF was added 4-methylmorpholine (2 mmol). The reaction mixture was then stirred at room temperature for 16 h. Water (12 mL) was then added to the mixture and the mixture was extracted with ethyl acetate. The organic layer was separated and washed with water and brine and then dried. Filtration and removal of solvent provided the crude product which was purified by flash chromatography (hexane/ethyl acetate) to yield the desired aryl sulfonamide amide analog.

### Example 50

### AD946

### (S)-4-0N-(1-Phenylpropyl)-4-(trifluoromethyl)phenylsulfonamido)methyl)benzoic acid

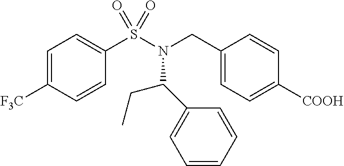

(S)-4-((N-(1-Phenylpropyl)-4-(trifluoromethyl)phenylsulfonamido)methyl)benzoic acid was prepared according to the General Method illustrated in Scheme 1.

MS (m/z): 477.0

Elemental Analysis: C₂₄H₂₂F₃NO₄S:

Calcd: C, 60.37%; H, 4.64%; N, 2.93%. Found: C, 60.60%; H4.75%; N2.97%

### Example 51

### AD947

### (R)-4-((4-Chloro-N-(2-hydroxy-1-phenylethyl)phenylsulfonamido)methyl)benzoic acid

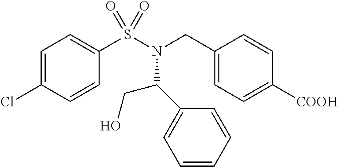

(R)-4-((4-Chloro-N-(2-hydroxy-1-phenylethyl)phenylsulfonamido)methyl)benzoic acid was prepared from Example 7 via hydrolysis described in the General Method illustrated in Scheme 1.

MS (m/z): 444.3

Mp 85-87° C.

### Example 52

### AD949

### (S)-4-((4-Chloro-N-(1-phenylpropyl)phenylsulfonamido)methyl)-3-fluorobenzoic acid

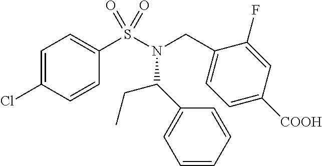

To a solution of (S)-4-chloro-N-(4-cyano-2-fluorobenzyl)-N-(1-phenylpropyl)benzenesulfonamide (100 mg) in ethanol (4 mL), NaOH (25 N, 0.268 mL) was added and the mixture was heated at 87° C. for 20 h. The reaction mixture was then cooled to room temperature and evaporated in vacuo. The residue was washed with ethyl ether (2 mL) and the aqueous layer was acidified by adding dropwise 6 N HCl until was pH 1-2 was reached. The resulting turbid solution was further diluted to 15 mL, extracted with ethyl acetate and the organic layer was then separated and dried with Na₂SO₄. After removing the ethyl acetate in vacuo, a solid product resulted in 65% yield.

MS (m/z): 461.1

Mp 118-120° C.

### Example 53

### AD958

### 4-((5-Chloro-N-(1,3-dihydroxypropan-2-yl)thiophene-2-sulfonamido)methyl)benzoic acid

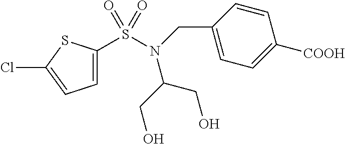 [SMILES: O=C(O)c1ccc(CN(C(CO)CO)S(=O)(=O)c2ccc(Cl)s2)cc1]

The title compound (55 mg, 67.7%) was prepared as a solid from methyl 4-((5-chloro-N-(2,2-dimethyl-1,3-dioxan-5-yl)thiophene-2-sulfonamido)methyl)benzoate (Example 42) according to the procedure described in Step 3 of Example 1.

MS (m/z): 460.0 (M⁺+1)

Elemental Analysis: C₁₅H₁₆ClNO₆S₂:

Calcd: C, 44.39; H, 3.97; N, 3.45. Found: C, 44.79; H, 4.21; N, 3.38 Mp 163-165° C.

### Example 54

### AD960

### 4-((4-Chloro-N-(1-(4-(trifluoromethyl)phenyl)propyl)phenylsulfonamido)methyl)benzoic acid

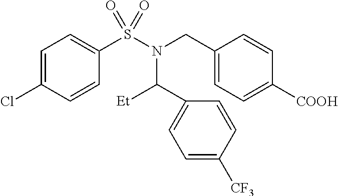 [SMILES: CCC(c1ccc(C(F)(F)F)cc1)N(Cc1ccc(C(=O)O)cc1)S(=O)(=O)c1ccc(Cl)cc1]

### Step 1

### Methyl 4-((4-chloro-N-(1-(4-(trifluoromethyl)phenyl)propyl)phenylsulfonamido)methyl)benzoate

To a solution of methyl 4-((4-chlorophenylsulfonamido)methyl)benzoate (179 mg, 0.526 mmol) and triphenylphosphine (276 mg, 1.053 mmol) in 5 mL of THF was added DIAD (0.230 ml, 1.158 mmol) dropwise. The reaction mixture was stirred at room temperature for 16 h. The solvent was then concentrated in vacuo and 10 mL of water was added to the residue followed by extraction with ethyl acetate. Evaporation of all solvent gave a crude product which was subjected to flash chromatography to yield a pure white solid (148.0 mg, 53% yield).

### Step 2

### 4-((4-Chloro-N-(1-(4-(trifluoromethyl)phenyl)propyl)phenylsulfonamido)methyl)benzoic acid

Methyl 4-((4-chloro-N-(1-(4-(trifluoromethyl)phenyl)propyl)phenylsulfonamido) methyl)benzoate (135 mg, 0.257 mmol) and potassium hydroxide (67.8 mg, 1.027 mmol) was suspended in 3 mL of MeOH and the mixture was stirred at 45° C. for 16 h. The solvent was concentrated in vacuo followed by the addition of 10 mL of water. The mixture was adjusted to pH 2, extracted with EtOAc and then purified via flash chromatography to give the title compound as a white solid (86 mg, 65% yield).

Elemental Analysis: (C₂₄H₂₁ ClF₃NO₄S):

Calcd: C, 56.31, H, 4.13, N, 2.74. Found C, 56.23, H, 3.83, N, 2.68 Mp 108-110° C.

### Example 55

### AD961

### Methyl 2-fluoro-4-((4-fluoro-N-(pentan-3-yl)phenylsulfonamido)methyl)benzoate

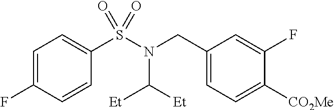 [SMILES: CCC(CC)N(Cc1ccc(C(=O)OC)c(F)c1)S(=O)(=O)c1ccc(F)cc1]

To a stirred solution of 4-fluoro-N-(pentan-3-yl)benzenesulfonamide (690 mg, 2.81 mmol) and methyl 4-(bromomethyl)-2-fluorobenzoate (834 mg, 3.38 mmol) in 6 mL of dry DMF was added K₂CO₃ at room temperature. The mixture was stirred for 20 h. The solvent was then evaporated followed by the addition of 10 mL of water. The resulting mixture was then extracted with EtOAc and purified by flash chromatography to give final product (509.0 mg, 44% yield).

¹H NMR (500 MHz, CDCl₃) δ7.88 (m, 2H), 7.26 (m, 3H), δ 7.17 (m, 2H), δ 4.35 (m, 2H), δ 3.99 (s, 3H), δ 3.56 (m, 1H), δ 1.36 (m, 2H), δ 1.18 (m, 2H), δ 0.72 (t, J=7 Hz, 6H)

### Example 56

### AD962

### (S)-Methyl 4-((4-chloro-N-(1-phenylpropyl)phenylsulfonamido)methyl)-2-fluorobenzoate

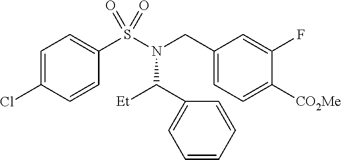

To a stirred solution of (S)-4-chloro-N-(1-phenylpropyl)benzenesulfonamide (0.310 g, 1 mmol) and methyl 4-(bromomethyl)-2-fluorobenzoate (0.296 g, 1.200 mmol) in 4 mL of dry DMF was added K₂CO₃ at room temperature. The mixture was then stirred for 20 h and the solvent was evaporated. Water (10 mL) was then added to the residue and the mixture was extracted with EtOAc. Purification by flash chromatography gave a white solid (185 mg, 38% yield).

Mp 96-98° C.

¹H NMR (500 MHz, CDCl₃) δ7.77 (m, 1H), δ 7.66 (m, 2H), δ 7.43 (m, 2H), δ 7.26 (m, 3H), δ 7.66 (m, 1H), δ 6.83-7.22 (m, 4H), δ 4.90 (dd, J1=10 Hz, J2=5 Hz, 1H), δ 4.42 (d, J=15 Hz, 1H), δ 4.08 (d, J=15 Hz, 1H), δ 3.91 (s, 3H), δ 1.83 (m, 1H), δ 1.71 (m, 1H), δ 0.77 (t, J=6 Hz, 3H)

### Example 57

### AD963

### (S)-4-Chloro-N-((5-cyanopyridin-2-yl)methyl)-N-(1-phenylpropyl)benzenesulfonamide

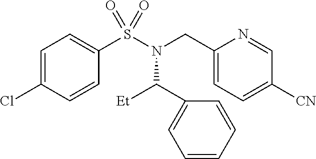

To a stirred solution of (S)-4-chloro-N-(1-phenylpropyl)benzenesulfonamide (0.310 g, 1 mmol) and 6-(bromomethyl)nicotinonitrile (0.236 g, 1.200 mmol) in dry DMF (4 mL) was added K₂CO₃ at room temperature. The mixture was then stirred for 20 h and the solvent was evaporated. Water (10 mL) was then added to the residue and the mixture was extracted with EtOAc. Purification by flash chromatography gave a solid (122.0 mg, 28.6% yield).

Elemental Analysis: C₂₂H₂₀ClN₃O₂S:

Calcd: C, 62.04, H, 4.73, N, 9.87. Found: C, 62.05, H, 4.64, N, 9.67

### Example 58

### AD964

### 4-Chloro-N-((5-cyanopyridin-2-yl)methyl)-N-(pentan-3-yl)benzenesulfonamide

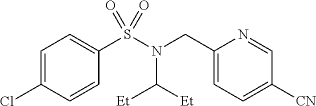 [SMILES: CCC(CC)N(Cc1ccc(C#N)cn1)S(=O)(=O)c1ccc(Cl)cc1]

To a stirred solution of 4-chloro-N-(pentan-3-yl)benzenesulfonamide (300 mg, 1.146 mmol) and 6-(bromomethyl)nicotinonitrile (271 mg, 1.375 mmol) in dry DMF (5 mL) was added K₂CO₃ at room temperature. The mixture was then stirred for 20 h and the solvent was evaporated. Water (10 mL) was then added to the residue and the mixture was extracted with EtOAc. Purification by flash chromatography gave a solid (59.0 mg, 13.6% yield).

Elemental Analysis: (C₁₈H₂₀ClN₃O₂S):

Calcd: C, 57.21, H, 5.33, N, 11.12. Found C, 57.52, H, 5.16, N, 11.33

### Example 59

### AD969

### (S)-3-((4-Chloro-N-(1-phenylpropyl)phenylsulfonamido)methyl)benzoic acid

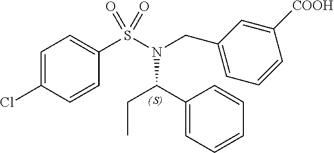

### Step 1

### (S)-Methyl 3-((4-chloro-N-(1-phenylpropyl)phenylsulfonamido)methyl)benzoate

To a mixture of (S)-4-chloro-N-(1-phenylpropyl)benzenesulfonamide and methyl 3-(bromomethyl)benzoate (247 mg, 1.07 mmol) in DMF (3 mL) was added Cs₂CO₃. The reaction mixture was stirred at room temperature for 16 h. To the reaction mixture was added water (12 mL) and then extracted with ethyl acetate. The organic layer was separated and washed with water, brine and dried over sodium sulfate to give 490 mg of crude product. The crude product was purified by flash chromatography (hexane:ether, 1-30%) and yielded 400 mg of desired product as a white solid.

MS m/z: 458.9 (M+1)

Elemental Analysis: C₂₄H₂₄ClNO₄S:

Calcd: C, 62.94; H, 5.18; N, 3.06. Found: C, 63.01; H, 5.25; N, 2.92

### Step 2

### (S)-3-((4-chloro-N-(1-phenylpropyl)phenylsulfonamido)methyl)benzoic acid

(S)-3-((4-chloro-N-(1-phenylpropyl)phenylsulfonamido)methyl)benzoic acid (180 mg, 71.4%) was prepared from 200 mg of (S)-methyl 3-((4-chloro-N-(1-phenylpropyl)phenylsulfonamido)methyl)benzoate according to the procedure described in Step 3 of Example 1.

MS (m/z): 426.09 (M⁺-OH)

Elemental Analysis: C₂₄H₂₄ClNO₅S:

Calcd: C, 62.23; H, 4.99; N, 3.16. Found: C, 62.08; H, 5.04; N, 3.14

Mp 65-67° C.

### Example 60

### AD975

### (S)-4-((4-Fluoro-N-(1-phenylpropyl)phenylsulfonamido)methyl)benzoic acid

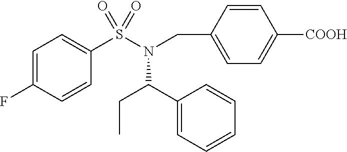

(S)-4-((4-Fluoro-N-(1-phenylpropyl)phenylsulfonamido)methyl)benzoic acid was prepared from (S)-methyl 4-((4-fluoro-N-(1-phenylpropyl)phenylsulfonamido)methyl)benzoate according to the General Method described in Scheme 1.

MS (m/z): 427.1

Elemental Analysis: C₂₃H₂₂FNO₄S:

Calcd: C, 64.62%, H, 5.19%, N, 3.28%. Found: C, 64.73%; H, 5.01%; N, 3.38%

### Example 61

### AD980

### Methyl 4-((4-chloro-N-(1,3-dihydroxypropan-2-yl)phenylsulfonamido)methyl)-benzoate

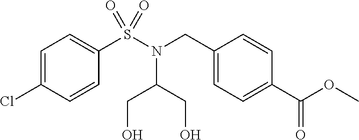 [SMILES: COC(=O)c1ccc(CN(C(CO)CO)S(=O)(=O)c2ccc(Cl)cc2)cc1]

### Step 1

### 4-Chloro-N-(2,2-dimethyl-1,3-dioxan-5-yl)benzenesulfonamide

To a solution of 2-aminopropane-1,3-diol (2.11 g, 23.16 mmol) in anhydrous THF (20 mL), potassium carbonate (7.62 g, 55 mmol) was added followed by the portion-wise addition of 4-chlorobenzene-1-sulfonyl chloride (4.66 g, 22.06 mmol). The reaction mixture was stirred 16 h. THF was removed in vacuo, the residue was partitioned between water (20 mL) and ethyl acetate (30 mL), and the organic layer was separated, washed with water, brine and dried. Filtration and removal of the solvent gave 4.556 g of crude product. The white solid was recrystallized in ethyl acetate to give 4.08 g (69.6%) of desired product.

MS (m/z): 266.9 (M⁺+1)

### Step 2

### Methyl 4-((4-chloro-N-(2,2-dimethyl-1,3-dioxan-5-yl)phenylsulfonamido)-methyl)-benzoate

To a mixture of 4-chloro-N-(2,2-dimethyl-1,3-dioxan-5-yl)benzenesulfonamide (250 mg, 0.818 mmol), methyl 4-(hydroxymethyl)benzoate (272 mg, 1.635 mmol) and triphenylphosphine (472 mg, 1.8 mmol) in THF (5 mL) was added DIAD (0.354 mL). The reaction mixture was stirred at room temperature for 16 h. THF was then removed in vacuo and the residue was purified by flash chromatography (ethyl acetate:dichloromethane, 5%) to yield 296 mg (80% yield) of desired product.

MS (m/z): 454.8 (M⁺+1)

### Step 3

### Methyl 4-((4-chloro-N-(1,3-dihydroxypropan-2-yl)phenylsulfonamido)methyl)-benzoate

A mixture of methyl 4-((4-chloro-N-(2,2-dimethyl-1,3-dioxan-5-yl)phenylsulfonamido)methyl)benzoate (195 mg, 0.430 mmol), 0.5 mL of methanol and 4-methylbenzenesulfonic acid hydrate (90 mg, 0.47 mmol) in 5 mL of THF was stirred at room temperature for 3 h. TLC indicated that the reaction was complete. Water (2 ml) and saturated aqueous sodium carbonate solution (1 mL) were added. The reaction mixture (now ph 11-12) was stirred at room temperature for 10 min. THF was removed in vacuo and the residue was partitioned between ethyl acetate and water. The organic layer was separated and washed with water and brine and dried over sodium sulfate. Concentration in vacuo gave 187 mg of crude product which was purified by flash chromatography (hexane/ethyl acetate: 0-60%) to yield 131 mg of desired product.

MS (m/z): 414.06 (M⁺+1)

Elemental Analysis: C₁₈H₂₀ClNO₆S:

Calcd: C, 52.24; H, 4.87; N, 3.38. Found: C, 52.30, H, 5.13, N, 3.30.

### Example 62

### AD983

### (S)-6-((4-Chloro-N-(1-phenylpropyl)phenylsulfonamido)methyl)nicotinic acid

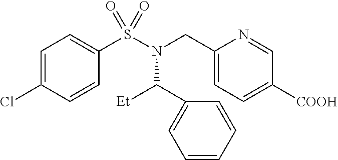

### Step 1

### (S)-Ethyl 6-((4-chloro-N-(1-phenylpropyl)phenylsulfonamido)methyl)nicotinate

To a stirred solution of (S)-4-chloro-N-(1-phenylpropyl)benzenesulfonamide (500 mg, 1.614 mmol) and ethyl 6-(bromomethyl)nicotinate (473 mg, 1.937 mmol) in dry DMF (6 mL) was added K₂CO₃ at room temperature. The mixture was then stirred for 24 h and the solvent was evaporated. Water (15 mL) was then added to the residue and the mixture was extracted with EtOAc. Purification by flash chromatography gave the desired product (522.0 mg, 68% yield).

### Step 2

### (S)-6-((4-Chloro-N-(1-phenylpropyl)phenylsulfonamido)methyl)nicotinic acid

To a solution of (S)-Ethyl 6-((4-chloro-N-(1-phenylpropyl)phenylsulfonamido)methyl)nicotinate (522 mg, 1.104 mmol), 1M NaOH (4.5 mL) solution was added. The mixture was stirred at room temperature for 16 h. The solvent was evaporated, water added and the solution was adjusted to pH 6. The mixture was then extracted with EtOAc and concentrated in vacuo to yield a white solid product (362.0 mg, 73% yield).

Mp 112-113° C.

¹H NMR (500 MHz, DMSO) δ7.79 (m, 2H), δ 7.59 (m, 2H), δ 7.48 (m, 1H), δ 7.20 (m, 3H), δ 7.12 (m, 2H), δ 6.85 (d, J=1 Hz, 1H), δ 6.68 (d, J=9 Hz, 1H), δ 4.82 (dd, J1=8 Hz, J2=5 Hz, 1H), δ 4.84 (d, J=14.5 Hz, 1H), δ 4.23 (d, J=14 Hz, 1H), δ 1.90 (m, 1H), δ 1.51 (m, 1H), δ 0.61 (t, J=6 Hz, 3H)

### Example 63

### AD988

### (S)-4-((4-Chloro-N-(1-phenylpropyl)phenylsulfonamido)methyl)-N-(4,4-dimethoxybutyl)benzamide

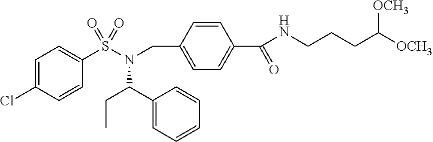

To a solution of (S)-4-((4-chloro-N-(1-phenylpropyl)phenylsulfonamido)methyl)-N-(4,4-dimethoxybutyl)benzamide in dichloromethane were added 4,4-dimethoxybutan-1-amine, N,N-methanediylidenedicyclohexanamine and 1H-benzo[d][1,2,3]triazol-1-ol. The solution was stirred for 16 h and then concentrated in vacuo to give crude product. Purification by flash chromatography gave the desired product in 30% yield.

MS (m/z): 587.04

Elemental Analysis: C₂₉H₃₅ClN₂O₅S:

Calcd: C, 62.30%; H, 6.31%; N, 5.01%. Found: C, 64.05%; H, 6.83%, N, 5.44%

### Example 64

### AD991

### (S)-2-(4-((4-Chloro-N-(1-phenylpropyl)phenylsulfonamido)methyl)benzamido)acetic acid

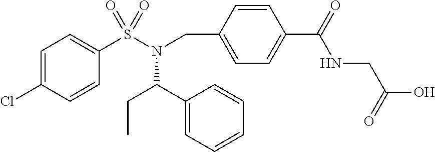

To the solution of (S)-4-((4-chloro-N-(1-phenylpropyl)phenylsulfonamido)methyl)-N-(2-hydroxyethyl)benzamide (130 mg, 0.267 mmol) in acetone (0.25 mL), chromium(VI) oxide (80 mg, 0.801 mmol) in sulfuric acid (1.5 M) was added dropwise and the reaction was stirred at room temperature for 5 h. The reaction mixture was concentrated in vacuo, acidified and then extracted with dichloromethane. Upon removal of the solvent, the residue was dissolved in diethyl ether and washed with 1N NaOH. This basic solution was acidified with 1N HCl to yield the desired product.

MS (m/z): 499.1

Mp 136-139° C.

### Example 65

### AD1012

### (S)-4-((4-Chloro-N-(1-phenylethyl)phenylsulfonamido)methyl)benzoic acid

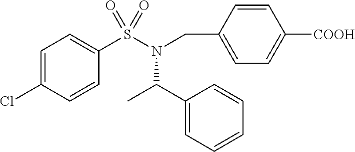

Synthesis of Example 65 was prepared by the hydrolysis of Example 12 according to the General Method described in Scheme 1.

### Example 66

### AD1020

### 4-((4-Chloro-N-(1,3-dihydroxypropan-2-yl)phenylsulfonamido)methyl)benzoic acid

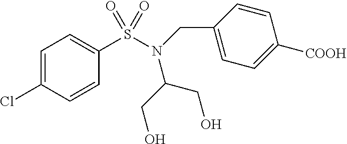 [SMILES: O=C(O)c1ccc(CN(C(CO)CO)S(=O)(=O)c2ccc(Cl)cc2)cc1]

To a mixture of methyl 4-((4-chloro-N-(1,3-dihydroxypropan-2-yl)phenylsulfonamido)-methyl)benzoate (51 mg, 0.123 mmol) in THF (5 mL), water (0.5 mL) and methanol (0.5 mL) was added lithium hydroxide monohydrate (31 mg). This reaction mixture was stirred at 45° C. for 2 h and then concentrated in vacuo. To the residue was added water (1.5 mL) and treated with 2N HCl to produce a precipitate. Ethyl acetate was added to extract the solid product. The organic layer was separated, washed with water and brine, concentrated in vacuo and dried to give a white solid product (48 mg, 97%).

MS (m/z): 399.22 (M⁺)

Elemental Analysis: C₁₇H₁₈ClNO₆S.H₂O:

Calcd: C, 48.86; H, 4.82; N, 3.35. Found: C, 49.04, H, 4.33, N, 3.13

### Example 67

### AD1022

### (S)-Methyl 4-((4-chloro-N-(1-phenylpropyl)benzamido)methyl)benzoate

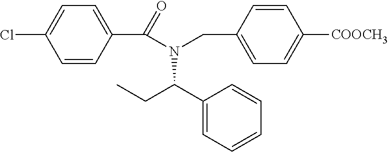 [SMILES: CC[C@@H](c1ccccc1)N(Cc1ccc(C(=O)OC)cc1)C(=O)c1ccc(Cl)cc1]

To a solution of (S)-methyl 4-((1-phenylpropylamino)methyl)benzoate (220 mg, 0.776 mmol) and triethylamine (236 mg, 2.329 mmol) in dichloromethane (2 mL), 4-chlorobenzoyl chloride (136 mg, 0.776 mmol) was added dropwise. The reaction mixture was stirred at room temperature for 16 h. The product was purified by flash chromatography to give the desired product in 65% yield.

### Example 68

### AD1023

### (S)-4-((4-Chloro-N-(1-phenylpropyl)phenylsulfonamido)methyl)-N-(2-methoxyethyl)benzamide

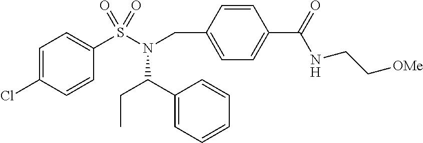

To the solution of (S)-4-((4-chloro-N-(1-phenylpropyl)phenylsulfonamido)methyl)-N-(2-hydroxyethyl)benzamide (165 mg, 0.339 mmol) in THF (2 mL) at 0° C., sodium hydride (24.39 mg, 1.016 mmol) in THF was added. After 30 min of stirring, iodomethane (48.1 mg, 0.339 mmol) was added to the reaction mixture. The mixture was then stirred at room temperature. The product was purified by flash chromatography to give the desired product in 42% yield.

MS (m/z): 501.1

Elemental Analysis: C₂₆H₂₉ClN₂O₄S:

Calcd: C, 62.33%; H, 5.83%; N, 5.59%. Found: C, 62.91%; H6.07%; N, 5.46%

### Example 69

### AD1025

### (S)-4-((4-Carboxy-N-(1-phenylpropyl)phenylsulfonamido)methyl)benzoic acid

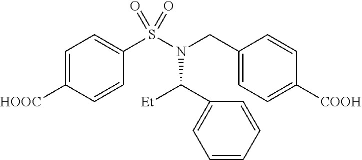 [SMILES: CC[C@@H](c1ccccc1)N(Cc1ccc(C(=O)O)cc1)S(=O)(=O)c1ccc(C(=O)O)cc1]

### Step 1

### (S)-Methyl 4-((4-(methoxycarbonyl)-N-(1-phenylpropyl)phenylsulfonamido)methyl)benzoate

To a stirred solution of (S)-methyl 4-(N-(1-phenylpropyl)sulfamoyl)benzoate (200 mg, 0.600 mmol) and methyl 4-(bromomethyl)benzoate (165 mg, 0.720 mmol) in dry

DMF (4 mL) was added K₂CO₃ at room temperature and the mixture was stirred for 16 h. The solvent was evaporated, water (20 mL) was added to the residue and then extracted with ethyl acetate. The layers were separated and the organic layer was concentrated in vacuo, dried and filtered and the solvent was removed. The crude product was then purified by flash chromatography to give the desired product. (141 mg, 49% yield).

### Step 2

### (S)-4-((4-carboxy-N-(1-phenylpropyl)phenylsulfonamido)methyl)benzoic acid

(S)-Methyl-4-(4-(methoxycarbonyl)-N-(1-phenylpropyl)phenylsulfonamido)methyl)benzoate (300 mg, 0.623 mmol) and potassium hydroxide (164 mg, 2.492 mmol) were stirred in MeOH (5 mL) at 50° C. for 3 h. The solvent was evaporated, water (5 mL) was added, and the resulting mixture was extracted with EtOAc (2×10 mL). The pH of the aqueous phase was then adjusted to pH 4-5 using 5N HCl solution. This mixture was then extracted with EtOAc, the solvent was evaporated and the white solid was then dried to give the desired product (75 mg, 26% yield).

Mp 102-104° C.

¹H NMR (400 MHz, CDCl₃) δ10.70 (s, 1H), δ 9.90 (s, 1H), δ 7.91 (m, 3H), δ 7.26-7.56 (m, 10H), δ 3.89 (m, 1H), δ 3.65 (m, J=12.8 Hz, 1H), δ 3.54 (d, J=13 Hz), δ 2.60 (m, 1H), δ 2.21 (m, 1H), δ 0.74 (t, J=6 Hz, 3H)

### Example 70

### AD1027

### 4-((4-Methyl-N-(pentan-3-yl)phenylsulfonamido)methyl)benzoic acid

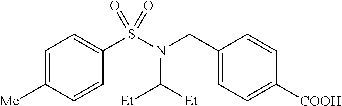 [SMILES: CCC(CC)N(Cc1ccc(C(=O)O)cc1)S(=O)(=O)c1ccc(C)cc1]

Methyl 4-((4-methyl-N-(pentan-3-yl)phenylsulfonamido)methyl)benzoate (350 mg, 0.899 mmol) and potassium hydroxide (237 mg, 3.59 mmol) were stirred in MeOH (5 mL) at 50° C. for 3 h. The solvent was evaporated, water (5 mL) was added, and the resulting mixture was extracted with EtOAc (2×10 mL). The pH of the aqueous phase was then adjusted to pH 4-5 using 5N HCl solution. This mixture was then extracted with EtOAc, the solvent was evaporated and the white solid was then dried to give the desired product (145 mg, 43% yield).

Mp 86-88° C.

¹H NMR (500 MHz, CDCl₃) δ7.99 (d, J=10.5 Hz, 2H), δ7.69 (d, J=10.5 Hz, 2H), δ7.47 (d, J=11. Hz, 2H), δ7.26 (d, J=10.5 Hz, 2H), δ 4.34 (s, 2H), δ 3.54 (m, 1H), δ 2.41 (s, 3H), δ 1.33 (m, 1H), δ 1.14 (m, 1H), δ 0.69 (t, J=6 Hz, 3H)

### Example 71

### AD1029

### 4-((4-Methoxy-N-(pentan-3-yl)phenylsulfonamido)methyl)benzoic acid

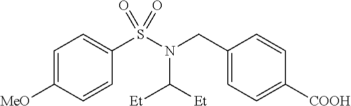 [SMILES: CCC(CC)N(Cc1ccc(C(=O)O)cc1)S(=O)(=O)c1ccc(OC)cc1]

Methyl 4-((4-methoxy-N-(pentan-3-yl)phenylsulfonamido)methyl)benzoate (230 mg, 0.567 mmol) and potassium hydroxide (150 mg, 2.269 mmol) were stirred at 50° C. in methanol for 3 h. The solvent was evaporated, water (5 mL) was added, and the resulting mixture was extracted with EtOAc (2×10 mL). The pH of the aqueous phase was then adjusted to pH 4-5 using 5N HCl solution. This mixture was then extracted with EtOAc, the solvent was evaporated and the white solid was then dried to give the desired product (113 mg, 51% yield).

Elemental Analysis: C₂₀H₂₅N₂O₅S:

Calcd: C, 61.36, H, 6.44, N, 3.58. Found C, 60.76, H, 6.60, N, 3.53

Mp 86-87° C.

### Example 72

### AD1031

### (S)—N-(4-(Aminomethyl)benzyl)-4-chloro-N-(1-phenylpropyl)benzenesulfonamide

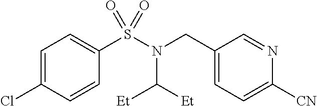 [SMILES: CCC(CC)N(Cc1ccc(C#N)nc1)S(=O)(=O)c1ccc(Cl)cc1]

4-Chloro-N-(pentan-3-yl)benzenesulfonamide (0.262 g, 1 mmol)₅-(bromomethyl)picolinonitrile (0.236 g, 1.200 mmol) and K₂CO₃ were stirred in DMF at room temperature for 16 h. The solvent was evaporated and water (10 mL) was added. The mixture was then extracted with EtOAc and the isolated crude product was purified by flash chromatography to yield the desired product (165 mg, 44% yield).

Elemental Analysis: C₁₈H₂₀ClN₃O₂S:

Calcd: C, 57.21, H, 5.33, N, 11.12. Found C, 57.27, H, 5.36, N, 11.06

Mp 108-110° C.

### Example 73

### AD1034

### N-((6-Cyanopyridin-3-yl)methyl)-4-fluoro-N-(pentan-3-yl)benzenesulfonamide

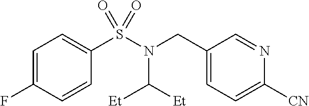 [SMILES: CCC(CC)N(Cc1ccc(C#N)nc1)S(=O)(=O)c1ccc(F)cc1]

4-Fluoro-N-(pentan-3-yl)benzenesulfonamide (0.245 g, 1 mmol), 5-(bromomethyl)picolinonitrile (0.236 g, 1.200 mmol) and K₂CO₃ were stirred in DMF at room temperature for 16 h. The solvent was evaporated and water (10 mL) was added. The mixture was then extracted with EtOAc and the isolated crude product was purified by flash chromatography to yield the desired product (137 mg, 38% yield).

Elemental Analysis: C₁₈H₂₀FN₃O₂S:

Calcd: C, 59.82, H, 5.58, N, 11.63. Found C, 59.89, H, 5.28, N, 11.37 Mp 118-120° C.

### Example 74

### AD1033

### (S)-4-Chloro-N-((6-cyanopyridin-3-yl)methyl)-N-(1-phenylpropyl)benzenesulfonamide

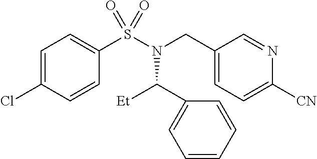 [SMILES: CC[C@@H](c1ccccc1)N(Cc1ccc(C#N)nc1)S(=O)(=O)c1ccc(Cl)cc1]

(S)-4-Chloro-N-(1-phenylpropyl)benzenesulfonamide (0.310 g, 1 mmol), 5-(bromomethyl)picolinonitrile (0.236 g, 1.200 mmol) and K₂CO₃ were stirred in DMF at room temperature for 16 h. The solvent was evaporated and water (10 mL) was added. The mixture was then extracted with EtOAc and the isolated crude product was purified by flash chromatography to yield the desired product (247 mg, 58% yield).

¹H NMR (500 MHz, CDCl₃) δ8.27 (d, J=1 Hz, 1H), δ7.71 (m, 2H), δ7.49 (m, 1H), 67.45 (m, 2H), 67.44 (d, J=6.5 Hz, 1H), δ 7.22 (m, 3H), δ 7.03 (m, 2H), 4.95 (dd, J1=14 Hz, J2=6 Hz, 1H), δ 4.37 (d, J=14. Hz, 1H), δ 4.30 (d, J=14 Hz, 1H), δ 3.97 (s, 3H), δ 1.90 (m, 1H), δ 1.64 (m, 1H), δ 0.77 (t, J=6 Hz, 3H).

### Example 75

### AD1034

### (S)-2-(4-((4-Chloro-N-(1-phenylpropyl)phenylsulfonamido)methyl)benzamido)-2-methylpropanoic acid

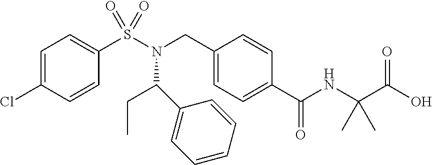

Example 75 was prepared by the procedure described for Example 64.

Elemental Analysis: C₂₇H₂₉ClN₂O₅S:

Calcd: C, 61.30%; H, 5.53%; N, 5.30%. Found: C, 60.98%; H5.12%; N5.14%

### Example 76

### AD1040

### (S)-4-Chloro-N-(4-(morpholine-4-carbonyl)benzyl)-N-(1-phenylpropyl)benzene-sulfonamide

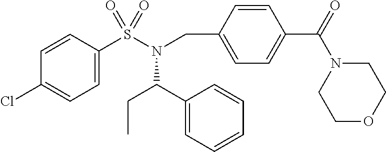

A solution of (S)-4-((4-chloro-N-(1-phenylpropyl)phenylsulfonamido)methyl)benzoic acid (222 mg, 0.5 mmol) in anhydrous THF (8 mL) was cooled to −50° C. and 4-methylmorpholine (0.066 ml, 0.600 mmol) was added dropwise. This mixture was stirred at −50° C. for 5 min. Isobutyl carbonochloridate (0.078 ml, 0.600 mmol) was then added dropwise and the mixture was stirred at −50° C. for 10 min, followed by the dropwise addition of morpholine (0.052 ml, 0.600 mmol). The mixture was allowed to warm to room temperature and stir for 3 h. THF was then removed in vacuo and the residue was partitioned between water and ethlyl acetate. The organic layer was separated and washed with water, brine and dried to give crude product. Purification by flash chromatography (hexane:ethyl acetate, 0-30%) yielded 82 mg of pure product.

MS (m/z): 513.13 (M⁺+1)

Elemental Analysis: C₂₇H₂₉ClN₂O₄S:

Calcd: C, 63.21; H, 5.70; N, 5.46. Found: C, 63.28, H, 5.99, N, 5.41

### Example 77

### AD1042

### (S)-Methyl 4-0N-(1-phenylpropyl)-6-(trifluoromethyl)pyridine-3-sulfonamido)-methyl)benzoate

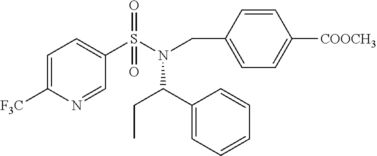

### Step 1

### (S)—N-(1-phenylpropyl)-6-(trifluoromethyl)pyridine-3-sulfonamide

To a solution of 6-(trifluoromethyl)pyridine-3-sulfonyl chloride (500 mg, 2.04 mmol) in THF (8 mL) was added potassium carbonate (1.125 g). (S)-1-phenylpropan-1-amine (289 mg, 2.138 mmol) was then added and the reaction mixture was stirred at room temperature for 6 h. The mixture was quenched with water (3 mL) and then THF was removed in vacuo. The resulting residue was extracted with ethyl acetate and the organic layer was then separated, washed with water and brine, filtered and dried to give 700 mg of white solid product. This solid was triturated with ether to give 631 mg of desired product.

MS (m/z): 345.7 (M⁺+1)

### Step 2

### (S)-Methyl 4-0N-(1-phenylpropyl)-6-(trifluoromethyl)pyridine-3-sulfonamido)-methyl)benzoate

A solution of (S)—N-(1-phenylpropyl)-6-(trifluoromethyl)pyridine-3-sulfonamide (586 mg, 1.702 mmol) and methyl 4-(bromomethyl)benzoate (429 mg, 1.872 mmol) in DMF (5 mL) was stirred with Cs₂CO₃ at room temperature for 16 h. Water (20 ml) was added to the mixture and extracted with ethyl acetate. The organic layer was separated and washed with water, brine and dried to give 700 mg of crude product. Purification by flash chromatography yielded 268 mg (32%) of desired product.

MS (m/z): 493.40 (M⁺+1)

Elemental Analysis: C₂₄H₂₃F₃N₂O₄S:

Calcd: C, 58.53; H, 4.71; N, 5.69. Found: C, 58.82, H, 4.67, N, 5.99

### Example 78

### AD1043

### (S)-4-0N-(1-phenylpropyl)-6-(trifluoromethyl)pyridine-3-sulfonamido)-methyl)benzoic acid

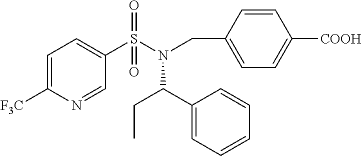

A solution of (S)-methyl 4-((N-(1-phenylpropyl)-6-(trifluoromethyl)pyridine-3-sulfonamido)methyl)benzoate (200 mg, 0.406 mmol) in THF (5 mL) was mixed with water (0.5 mL) and methanol (0.5 mL). Lithium hydroxide monohydrate (102 mg, 2.4 mmol) was then added and the reaction mixture was stirred at room temperature for 16 h. Water (1 mL) was added to the reaction mixture and then 2N HCl was used to adjust the pH of the mixture to pH 2. THF was then removed in vacuo upon which a white solid precipitated out and was filtered. The solid was washed with water and hexane, and dried to give 189 mg (97%) of desired product.

MS (m/z): 479.10 (M⁺+1)

Elemental Analysis: C₂₃H₂₁F₃N₂O₄S:

Calcd: C, 57.73; H, 4.42; N, 5.85. Found: C, 57.45, H, 4.45, N, 5.73

### Example 79

### AD1045

### 4-((4-Chloro-N—((S)-1-phenylpropyl)phenylsulfonamido)methyl)-N-((R)-2-hydroxypropyl)benzamide

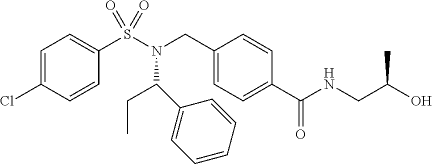

Example 79 was prepared by the procedure described for Example 5.

MS (m/z): 501.4

Elemental Analysis: C₂₆H₂₉ClN₂O₄S:

Calcd: C, 62.33%, H, 5.83%, N, 5.59%. Found: C, 62.07%, H, 5.53%, N, 5.57%

### Example 80

### AD1046

### 4-((4-Chloro-N—((S)-1-phenylpropyl)phenylsulfonamido)methyl)-N-((S)-2-hydroxypropyl)benzamide

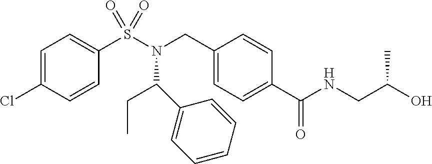

Example 80 was prepared by the procedure described for Example 5.

MS (m/z): 501.3

Elemental Analysis: C₂₆H₂₉ClN₂O₄S:

Calcd: C, 62.33%, H, 5.83%, N, 5.59%. Found: C, 62.05%, H, 5.83%, N, 5.59% Mp 78-80° C.

### Example 81

### AD1047

### (S)-4-((4-Chloro-N-(1-phenylpropyl)phenylsulfonamido)methyl)-N-(2-oxopropyl)benzamide

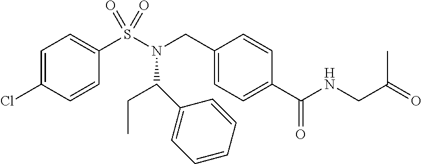

Chromium(VI) oxide (70 mg, 0.700 mmol) in sulfuric acid (0.176 mL, 0.527 mmol) was slowly added to a solution of 4-((4-chloro-N—((S)-1-phenylpropyl)phenylsulfonamido)methyl)-N-((R)-2-hydroxypropyl)benzamide (120 mg, 0.240 mmol) in acetone (3 mL). After stirring at room temperature for 16 h, the solution was diluted with ethyl acetate and washed with a NaHCO₃ aqueous solution. The organic layer was separated, washed with brine and then concentrated in vacuo to give crude product which was purified by flash chromatography to give the desired product in 52% yield.

MS (m/z): 499.2

### Example 82

### AD1048

### (R)-Methyl 2-(4-((4-chloro-N—((S)-1-phenylpropyl)phenylsulfonamido)methyl)-benzamido)-3-hydroxypropanoate

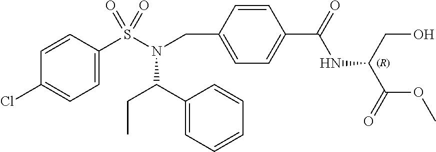

The title compound was obtained according to the General Procedure for Synthesis of Amide of Example 1. To a mixture of (S)-4-((4-chloro-N-(1-phenylpropyl)phenylsulfonamido)methyl)-benzoic acid (351 mg, 0.791 mmol), (R)-methyl 2-amino-3-hydroxypropanoate —HCl (148 mg, 0.949 mmol), EDC (182 mg, 0.949 mmol) and HATU [0-(7-Azabenzotriazol-1-yl)-N,N,N′,N′-tetramethyluronium hexafluorophosphate (361 mg, 0.949 mmol)] in anhydrous DMF (2 mL) was added 4-methylmorpholine (0.174 ml, 1.581 mmol) and stirred at room temperature for 16 h. Water (12 mL) was added and the mixture was then extracted with ethyl acetate. The organic layer was separated and washed with water, brine and dried. After drying, 378 mg of crude product was collected and purified by flash chromatography (hexane:ethyl acetate, 0-80%, 20 min) to yield 284 mg (65.9%) of the title compound.

MS (m/z): 545.12 (M⁺+1)

Elemental Analysis: C₂₇H₂₉ClN₂O₆S:

Calcd: C, 59.50; H, 5.36; N, 5.14. Found: C, 59.29, H, 5.28, N, 5.03

### Example 83

### AD1049

### (S)-4-((4-Chloro-N-(1-phenylpropyl)phenylsulfonamido)methyl)-N-(2-(methylthio)-ethylbenzamide

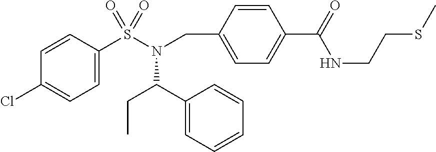

The title compound (214 mg, 61%) was obtained from (S)-4-((4-chloro-N-(1-phenylpropyl)phenylsulfonamido)methyl)benzoic (300 mg, 0.676 mmol) and 2-(methylthio)ethanamine (0.075 ml, 0.811 mmol) according to the General Procedure for Synthesis of Amide of Example 1.

MS (m/z): 517.20 (M⁺+1)

Elemental Analysis: C₂₆H₂₉ClN₂O₃S₂:

Calcd: C, 60.39; H, 5.65; N, 5.42. Found: C, 60.24, H, 5.93, N, 5.36

### Example 84

### AD1054

### (S)-4-Chloro-N-(4-cyanobenzyl)-N-(1-(4-cyanophenyl)propyl)benzenesulfonamide

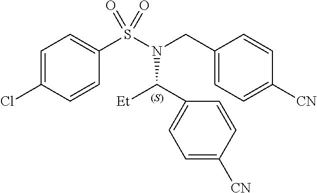

(S)-4-Chloro-N-(1-(4-cyanophenyl)propyl)benzenesulfonamide (0.670 g, 2 mmol), 4-(bromomethyl)benzonitrile (0.471 g, 2.400 mmol), and K₂CO₃ were stirred at room temperature in DMF for 16 h. The solvent was evaporated, water (10 mL) was added and the mixture was then extracted with EtOAc. After usual work-up, purification of the crude product by flash chromatography yielded a white solid (530 mg, 58% yield) as the final product.

Elemental Analysis: C₂₄H₂₀ClN₃O₂S:

Calcd: C, 64.06, H, 4.48, N, 9.34. Found C, 63.90, H, 4.15, N, 9.18. Mp 120-121° C.

### Example 85

### AD1056

### (S)-4-((4-Chloro-N-(1-phenylpropyl)phenylsulfonamido)methyl)-N-morpholinobenzamide

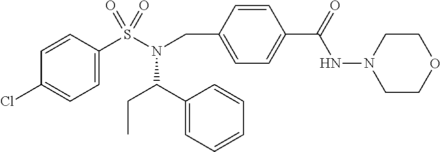

A solution of (S)-4-((4-chloro-N-(1-phenylpropyl)phenylsulfonamido)methyl)benzoic acid (220 mg, 0.496 mmol) in THF (8 mL) was cooled to −50° C. and 4-methylmorpholine (0.065 ml, 0.595 mmol) was added dropwise. The reaction mixture was stirred for 5 min and then isobutyl carbonochloridate (0.078 ml, 0.595 mmol) was added dropwise. The reaction mixture was stirred at −40° C. for 10 min 4-methylmorpholine (0.065 ml, 0.595 mmol) was added again. After stirring for 5 min, morpholin-4-amine (0.057 ml, 0.595 mmol) was added dropwise. The mixture was stirred for 45 min at −40° C., and then at room temperature for 4.5 h. THF was then removed in vacuo and the residue was partitioned between water and ethyl acetate. The organic layer was separated and washed with water, brine and dried. After work up, 284 mg crude product was collected and purified by flash chromatography (hexane:ethyl acetate, 0-90%) to yield 108 mg (41.3%) of title compound.

MS (m/z): 528.17 (M⁺+1)

Elemental Analysis: (C₂₇H₃₀ClN₃O₄S):

Calcd: C, 61.41; H, 5.73; N, 7.96. Found: C, 61.57, H, 5.72, N, 7.95

### Example 86

### AD1057

### (S)-4-((4-Chloro-N-(1-phenylpropyl)phenylsulfonamido)methyl)-N-(1,3-dihydroxypropan-2-yl)benzamide

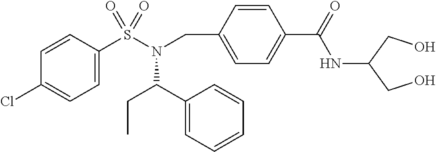

The title compound (172 mg, 59%) was obtained from (S)-4-((4-chloro-N-(1-phenylpropyl)phenylsulfonamido)methyl)benzoic acid (250 mg, 0.56 mmol) and 2-aminopropane-1,3-diol (61.6 mg, 0.676 mmol) according to the General Procedure for Synthesis of Amide of Example 1.

MS (m/z): 517.21 (M⁺+1)

Elemental Analysis: C₂₆H₂₉ClN₂O₅S:

Calcd: C, 60.40; H, 5.65; N, 5.42. Found: C, 59.99, H, 5.80, N, 5.66

### Example 87

### AD1058

### (R)-2-(4-((4-Chloro-N—((S)-1-phenylpropyl)phenylsulfonamido)methyl)benzamido)-3-hydroxypropanoic acid

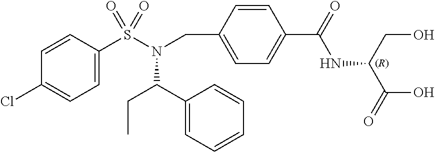

To a solution of (R)-methyl 2-(4-((4-chloro-N—((S)-1-phenylpropyl)phenylsulfonamido)methyl)benzamido)-3-hydroxypropanoate (214 mg, 0.393 mmol) in THF (5 mL) and water (1 mL), LiOH hydrate was added. The mixture was stirred at room temperature for 16 h. THF was removed in vacuo and water (1 mL) was added. The reaction mixture was acidified with 4N HCl to pH 2 and then extracted with ethyl acetate. The organic layer was separated, washed with water and brine, dried and then filtered. Removal of solvent provided 190 mg (91%) of the desired product.

MS (m/z): 531.13 (M⁺+1)

Elemental Analysis: C₂₆H₂₇ClN₂O₆S: Calcd: C, 58.81; H, 5.12; N, 5.28. Found: C, 59.32, H, 5.16, N, 5.02

### Example 88

### AD1059

### (S)-4-((4-Chloro-N-(1-phenylpropyl)phenylsulfonamido)methyl)-N-(2-(methylsulfonyl)ethyl)benzamide

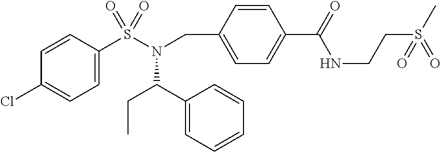

A mixture of (S)-4-((4-chloro-N-(1-phenylpropyl)phenylsulfonamido)methyl)-N-(2-(methylthio)ethyl)benzamide (156 mg, 0.302 mmol) and 3-chlorobenzoperoxoic acid (203 mg, 0.905 mmol) in anhydrous dichloromethane (5 mL) was stirred at room temperature for 3 h. Dimethyl sulfoxide (64.3 μA, 0.905 mmol) was added and stirred for 10 min. Dicholoromethane was removed in vacuo and the residue was partitioned between water and ethyl acetate. Sodium carbonate solution was added to bring the pH of the mixture to pH 11. The organic layer was separated and washed with water and brine and dried. After work up, 187 mg of crude product was collected and purified by flash chromatography (hexane:ethyl acetate, 0-90%) to yield 122 mg (73.6%) of desired product.

MS (m/z): 549.09 (M⁺+1)

Elemental Analysis: C₂₆H₂₉ClN₂O₅S₂:

Calcd: C, 56.87; H, 5.32; N, 5.10. Found: C, 56.62, H, 5.24, N, 5.00

### Example 89

### AD1063

### 4-((4-Chloro-N-(1-(6-chloropyridin-3-yl)propyl)phenylsulfonamido)methyl)benzoic acid

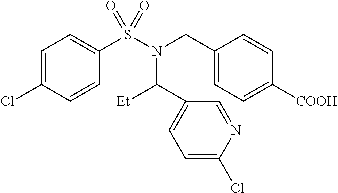 [SMILES: CCC(c1ccc(Cl)nc1)N(Cc1ccc(C(=O)O)cc1)S(=O)(=O)c1ccc(Cl)cc1]

Methyl-((4-chloro-N-(1-(6-chloropyridin-3-yl)propyl)phenylsulfonamido)methyl)-benzoate (790 mg, 1.601 mmol) and lithium hydroxide hydrate (202 mg, 4.80 mmol) were stirred at 50° C. for 16 h. THF was concentrated in vacuo, water (10 mL) was added and the mixture was extracted with EtOAc. Purification using flash chromatography yielded a white solid product (652 mg, 85% yield).

Elemental Analysis: C₂₂H₂₀Cl₂N₂O₄S:

Calcd: C, 55.12, H, 4.21, N, 5.84. Found C, 55.31; H, 4.08, N, 5.52

Mp 111-113° C.

### Example 90

### AD1065

### 4-((4-Chloro-N—((S)-1-phenylpropyl)phenylsulfonamido)methyl)-N-((2R,3R)-1,3-dihydroxybutan-2-yl)benzamide

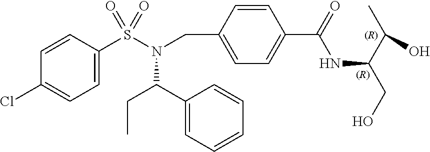

The title compound (172 mg, 59%) was obtained from (S)-4-((4-chloro-N-(1-phenylpropyl)phenylsulfonamido)methyl)benzoic acid (222 mg, 0.5 mmol) and 2-(2R,3R)-2-aminobutane-1,3-diol (L-threoninol, 63.1 mg, 0.600 mmol) according to the General Procedure for Synthesis of Amide of Example 1.

MS (m/z): 531.20 (M⁺+1)

Elemental Analysis: C₂₇H₃₁ClN₂O₅S):

Calcd: C, 61.06; H, 5.88; N, 5.27. Found: C, 61.33, H, 5.68, N, 5.46

### Example 91

### AD1066

### 4-((4-Chloro-N—((S)-1-phenylpropyl)phenylsulfonamido)methyl)-N-((2S,3S)-1,3-dihydroxybutan-2-yl)benzamide

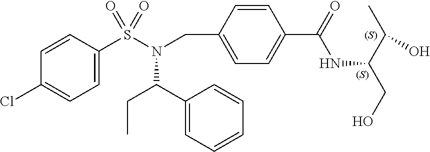

The title compound (189 mg, 71%) was obtained from of (S)-4-((4-chloro-N-(1-phenylpropyl)phenylsulfonamido)methyl)benzoic acid (222 mg, 0.5 mmol) and 2-(2S,3S)-2-aminobutane-1,3-diol (R-threoninol, 63.1 mg, 0.600 mmol) according to the General Procedure for Synthesis of Amide of Example 1.

MS (m/z): 531.4 (M⁺+1)

Elemental Analysis: C₂₇H₃₁ClN₂O₅S:

Calcd: C, 61.06; H, 5.88; N, 5.27. Found: C, 61.28, H, 5.71, N, 5.36.

### Example 92

### AD1067

### 4-((4-Chloro-N—((S)-1-phenylpropyl)phenylsulfonamido)methyl)-N-(3,3,3-trifluoro-2-hydroxypropyl)benzamide

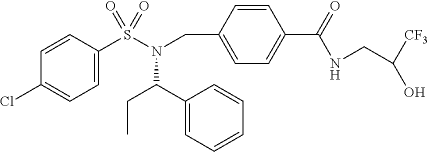

The title compound (115 mg, 41%) was obtained from of (S)-4-((4-chloro-N-(1-phenylpropyl)phenylsulfonamido)methyl)benzoic acid (222 mg, 0.5 mmol) and 3-amino-1,1,1-trifluoropropan-2-ol (77 mg, 0.600 mmol) according to the General Procedure for Synthesis of Amide of Example 1.

MS (m/z): 555.31 (M⁺+1)

Elemental Analysis: C₂₆H₂₆ClF₃N₂O₄S:

Calcd: C, 56.27; H, 4.72; N, 5.05. Found: C, 55.98, H, 4.71, N, 5.16

### Example 93

### AD1068

### (S)-4-((4-Chloro-N-(1-phenylpropyl)phenylsulfonamido)methyl)-N-((tetrahydro-2H-pyran-4-yl)methyl)benzamide

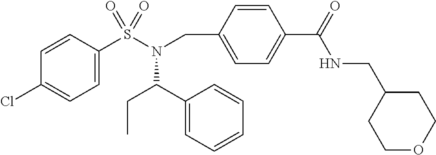

The title compound (195 mg, 72%) was obtained from (S)-4-((4-chloro-N-(1-phenylpropyl)phenylsulfonamido)methyl)benzoic acid (222 mg, 0.5 mmol) and (tetrahydro-2H-pyran-4-yl)methanamine hydrochloric salt (91 mg, 0.600 mmol) according to the General Procedure for Synthesis of Amide of Example 1.

MS (m/z): 541.32 (M⁺+1)

Elemental Analysis: C₂₉H₃₃ClN₂O₄S:

Calcd: C, 64.37; H, 6.15; N, 5.18. Found: C, 64.58, H, 6.21, N, 5.26

### Example 94

### AD1069

### (R)-Methyl 4-((4-chloro-N-(1-hydroxy-3-phenylpropan-2-yl)phenylsulfonamido)-methyl)benzoate

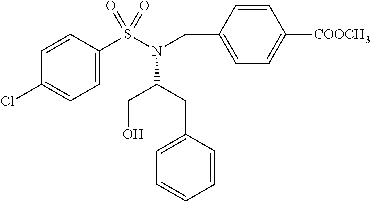

### Step 1

### (R)-4-Chloro-N-(1-hydroxy-3-phenylpropan-2-yl)benzenesulfonamide

To a solution of (R)-(+)-2-amino-3-phenyl-1-propanol (984 mg, 3.3 mmol) in dichloromethane (18 mL) was added triethyl amine (2.50 mL, 9 mmol) followed by 4-chloro-phenylsulfonayl chloride (1.25 g, 3 mmol). The mixture was stirred at room temperature for 16 h and then acidified with 2 N HCl to pH 3.5. The organic layer was separated and washed with water, aqueous sodium bicarbonate, water and brine and then dried over sodium sulfate. Filtration and concentration in vacuo provided 2.50 g of product.

MS (m/z): 325.32 (M⁺)

### Step 2

### (R)-Methyl 4-((4-chloro-N-(1-hydroxy-3-phenylpropan-2-yl)phenylsulfonamido)-methyl)benzoate

Following the procedure described in Step 2 of Example 1, (R)-4-Chloro-N-(1-hydroxy-3-phenylpropan-2-yl)benzenesulfonamide (1.0 g, 3.2 mmol) and methyl-4-bromomethylbenzoate (0.76 g, 3.3 mmol) yielded 1.189 g (78.2%) of desired product.

MS (m/z): 473.3 (M⁺)

Mp 128-130° C.

### Example 95

### AD1071

### (S)-4-((4-Chloro-N-(2-hydroxy-1-phenylethyl)phenylsulfonamido)methyl)benzoic acid

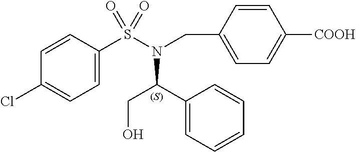

### Step 1

### (S)-4-Chloro-N-(2-hydroxy-1-phenylethyl)benzenesulfonamide

To a solution of (S)-(+)-phenyl-glycinol (1.0 g, 7.3 mmol) in dichloromethane (30 mL) was added triethyl amine (2.89 ml, 20.8 mmol). The mixture was then added to 4-chloro-phenylsulfonayl chloride (1.46 g, 6.9 mmol), stirred at room temperature for 16 h, followed by acidification with 2 N HCl to pH 3. The organic layer was separated and washed with water, aqueous sodium bicarbonate, water, brine and dried over sodium sulfate. Filtration and concentration provided 1.69 g of solid crude product that was triturated with diethyl ether to yield 1.31 g (60.8%) of white solid.

MS: (m/z) 312.14 (M⁺+1)

### Step 2

### (S)-methyl 4-((4-Chloro-N-(2-hydroxy-1-phenylethyl)phenylsulfonamido)-methyl)-benzoate

According to the procedure described in Step 2 of Example 1, (S)-4-chloro-N-(2-hydroxy-1-phenylethyl)benzenesulfonamide (800 mg, 2.56 mmol) and methyl-4-bromomethylbenzoate (0.635 g, 2.77 mmol) yielded 0.82 g (70%) white solid.

MS (m/z): 460.13 (M⁺+1)

Mp: 109-111° C.

### Step 3

### (S)-4-((4-Chloro-N-(2-hydroxy-1-phenylethyl)phenylsulfonamido)methyl)benzoic acid

(S)-Methyl 4-((4-chloro-N-(2-hydroxy-1-phenylethyl)phenylsulfonamido)methyl)-benzoate (220 mg, 0.49 mmpl) was hydrolyzed according to the procedure described in Step 3 of Example 1 to give 167 mg (76.5%) of desired product.

MS (m/z): 446.1 (M⁺+1)

Elemental Analysis: C₂₂H₂₀ClNO₅S.½H₂O:

Calcd: C, 58.08; H, 4.65; N, 3.08. Found: C, 58.47, H, 4.39, N, 3.12 Mp 86-88° C.

### Example 96

### AD1072

### Methyl 4-((4-chloro-N-((2S,3S)-1,3-dihydroxybutan-2-yl)phenylsulfonamido)-methyl)benzoate

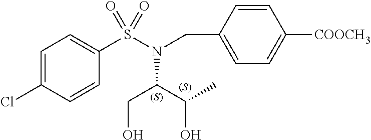

### Step 1

### 4-Chloro-N-((2S,3S)-1,3-dihydroxybutan-2-yl)benzenesulfonamide

A mixture of 4-chlorobenzene-1-sulfonyl chloride (3.82 g, 18.12 mmol), (2S,3S)-2-aminobutane-1,3-diol (D-threoninol, 2.0 g, 19.02 mmol) and potassium carbonate in anhydrous THF (20 mL) was stirred for 16 h. THF was removed in vacuo and the residue was partitioned between water (20 mL) and ethyl acetate (30 mL). The organic layer was separated and washed with 2N HCl, water, 10% sodium bicarbonate solution, water, brine and dried. Filtration and removal of solvent provided 4.30 g of final product.

MS (m/z): 280.42 (M⁺+1)

### Step 2

### Methyl 4-((4-chloro-N-((2S,3S)-1,3-dihydroxybutan-2-yl)phenylsulfonamido)-methyl)benzoate

4-Chloro-N-((2S,3S)-1,3-dihydroxybutan-2-yl)benzenesulfonamide (559 mg, 2 mmol) was reacted with methyl-4-bromomethylbenzoate (481 mg, 2.1 mmol) according to the procedure described in Step 2 of Example 1 to yield a white solid (741 mg, 86%) as the desired product.

MS (M/Z): 428.17 (M⁺+1)

Elemental Analysis: C₁₉H₂₂ClNO₆S:

Calcd: C, 53.33; H, 5.18; N, 3.27. Found: C, 53.33, H, 5.12, N, 3.17

### Example 97

### AD1074

### (S)-4-Chloro-N-(3-fluoro-4-methoxybenzyl)-N-(1-phenylpropyl)benzenesulfonamide

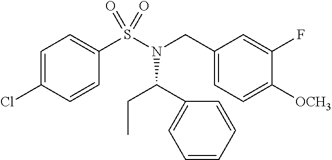 [SMILES: CC[C@@H](c1ccccc1)N(Cc1ccc(OC)c(F)c1)S(=O)(=O)c1ccc(Cl)cc1]

To a mixture of (S)-4-chloro-N-(1-phenylpropyl)benzenesulfonamide (309 mg, 1 mmol) see Example 1) and 3-fluoro-4-methoxybenzylbromide (230 mg, 1.05 mmol) in DMF (2 mL) was added 653 mg of cesium carbonate. The mixture was stirred for 16 h and then treated with water (12 mL) and ethyl acetate (10 mL). The organic layer was separated and washed with water, brine and dried over sodium sulfate. Filtration and concentration in vacuo provided 383 mg of crude product which was purified by flash chromatography to provide 217 mg (48%) of desired product.

MS (m/z): 448.17 (M⁺+1)

Elemental Analysis: C₂₃H₂₃ClFNO₃S:

Calcd: C, 61.67; H, 5.18; N, 3.13. Found: C, 62.01, H, 5.12, N, 3.21

### Example 98

### AD1075

### (S)-4-Chloro-N-(2,3-difluoro-4-methoxybenzyl)-N-(1-phenylpropyl)benzene-sulfonamide

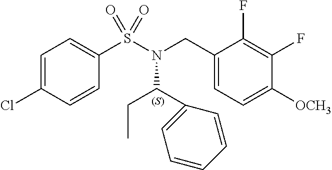

A mixture of (S)-4-chloro-N-(1-phenylpropyl)benzenesulfonamide (186 mg, 0.600 mmol), 1-(bromomethyl)-2,3-difluoro-4-methoxybenzene (157 mg, 0.660 mmol) and cesium carbonate (391 mg, 2 mmol) in DMF (3 mL) was stirred at room temperature for 4 hours. Water (30 mL) was added and the product was extracted with ethyl acetate. The organic layer was separated and washed with water, brine and dried. Removal of solvent and sodium sulfate provided 292 mg of crude product which was purified by flash chromatography (hexane:ethyl acetate, 0-20%) to provide 231 mg (83%) of final product.

MS (m/z): 466.19 (M⁺+1)

Elemental Analysis: C₂₃H₂₂ClF₂NO₃S:

Calcd: C, 59.29; H, 4.76; N, 3.01. Found: C, 59.46, H, 4.93, N, 3.31

### Example 99

### AD1077

### (S)-Methyl 2-(4-((4-chloro-N—((S)-1-phenylpropyl)phenylsulfonamido)methyl)-benzamido)-3-hydroxypropanoate

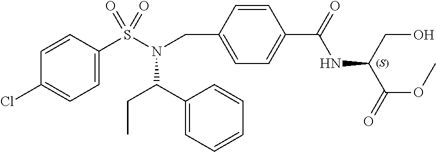

The title compound was prepared from (S)-4-((4-chloro-N-(1-phenylpropyl)phenyl-sulfonamido)methyl)benzoic acid (351 mg, 0.791 mmol) and L-serine methyl ester (148 mg, 0.95 mmol) according to the General Procedure for Synthesis of Amide of Example 1. White solid product (301 mg, 69.8%) was obtained.

MS (m/z): 545.12 (M⁺+1)

Elemental Analysis: C₂₇H₂₉ClN₂O₆S:

Calcd: C, 59.50; H, 5.36; N, 5.14. Found: C, 59.21, H, 5.46, N, 5.09

### Example 100

### AD1078

### (S)-2-(4-((4-Chloro-N—((S)-1-phenylpropyl)phenylsulfonamido)methyl)benzamido)-3-hydroxypropanoic acid

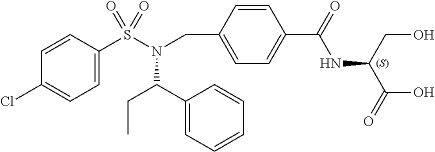

(S)-Methyl 2-(4-((4-chloro-N-((S)-1-phenylpropyl)phenylsulfonamido)methyl)-benzamido)-3-hydroxypropanoate (215 mg, 0.4 mmol) was hydrolyzed according to the procedure described in Step 3, Example 1. A white powder (203 mg, 97%) was generated as the product.

MS (m/z): 531.13 (M⁺+1)

Elemental Analysis: C₂₆H₂₇ClN₂O₆S:

Calcd: C, 58.81; H, 5.12; N, 5.28. Found: C, 58.54, H, 5.39, N, 5.46

### Example 101

### AD1082

### 4-((4-Chloro-N—((S)-1-phenylpropyl)phenylsulfonamido)methyl)-N-((S)-1-methoxypropan-2-yl)benzamide

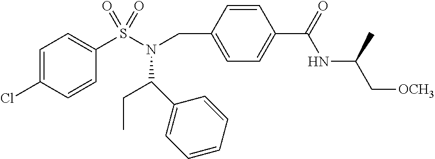

Example 101 was prepared by the procedure described for Example 68.

MS (m/z): 515.2

### Example 102

### AD1084

### (S)-4-((4-Chloro-N-(1-phenylpropyl)phenylsulfonamido)methyl)-N-isopropylbenzamide

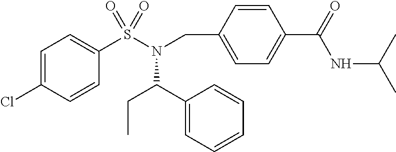

To a solution of (S)-4-((4-chloro-N-(1-phenylpropyl)phenylsulfonamido)methyl)benzoic acid (100 mg, 0.225 mmol), propan-2-amine (0.021 mL, 0.248 mmol) and 1H-benzo[d][1,2,3]triazol-1-ol (33.5 mg, 0.248 mmol) in dichloromethane (2 mL) was added DCC (51.1 mg, 0.248 mmol) and the reaction mixture was stirred at room temperature for 5 h. The reaction mixture was then filtered and the filtrate was diluted with ethyl acetate and brine. The organic layer was concentrated in vacuo, dried, and purified by flash chromatography to give the title compound.

MS (m/z): 484.1

Elemental Analysis: C₂₆H₂₉ClN₂O₃S:

Calcd: C, 64.38%; H, 6.03%; N, 5.78%. Found: C, 64.03, H, 6.38%, N, 6.36%

### Example 103

### AD1089

### (S)-4-((4-Chloro-N-(1-(4-cyanophenyl)propyl)phenylsulfonamido)methyl)benzoic acid

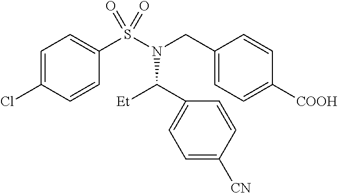 [SMILES: CC[C@@H](c1ccc(C#N)cc1)N(Cc1ccc(C(=O)O)cc1)S(=O)(=O)c1ccc(Cl)cc1]

(S)-Methyl 4-((4-chloro-N-(1-(4-cyanophenyl)propyl)phenylsulfonamido)methyl)-benzoate (2.10 g, 4.35 mmol) and lithium hydroxide hydrate (0.547 g, 13.04 mmol) were stirred at 50° C. for 16 h. THF was removed, water (20 mL) was added, and the mixture was then extracted with ether to remove impurities. The pH of the mixture was adjusted to pH 2 and then extracted with EtOAc, dried, filtered and concentrated in vacuo to give a white solid (1.2 g, 59% yield).

Elemental Analysis: C₂₄H₂₁ClFN₂O₄S:

Calcd: C, 61.47, H, 4.51, N, 5.97. Found C, 61.69, H, 4.58, N, 5.37 Mp 132-133° C.

### Example 104

### AD1090

### 4-Chloro-N-(4-((R)-2-(hydroxymethyl)pyrrolidine-1-carbonyl)benzyl)-N-((S)-1-phenylpropyl)benzenesulfonamide

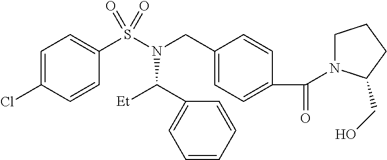 [SMILES: CC[C@@H](c1ccccc1)N(Cc1ccc(C(=O)N2CCC[C@@H]2CO)cc1)S(=O)(=O)c1ccc(Cl)cc1]

(S)-4-((4-Chloro-N-(1-phenylpropyl)phenylsulfonamido)methyl)benzoicacid (0.222 g, 0.5 mmol) and triethylamine (0.209 ml, 1.500 mmol) were stirred at −20° C. in DCM (2 mL), Methanesulfonyl chloride (0.039 ml, 0.500 mmol) was added and the mixture was stirred at −20° C. for 1 h. (S)-(+)-2-Pyrrolidinemethanol (0.061 ml, 0.610 mmol) was then added and the mixture was stirred at −20° C. for 1 h and warmed up to room temperature and stirred for 16 h. The solvent was evaporated, water (5 mL) was added and the mixture was extracted with EtOAc. Purification by flash chromatography gave a white solid product (160 mg, 61% yield).

Elemental Analysis: C₂₈H₃₁ ClN₂O₄S:

Calcd: C, 63.80, H, 5.93, N, 5.31. Found C, 63.60, H, 6.06, N, 5.12

Mp 120-121° C.

### Example 105

### AD1096

### 4-((4-Chloro-N—((S)-1-phenylpropyl)phenylsulfonamido)methyl)-N-((1r,4r)-4-hydroxycyclohexyl)benzamide

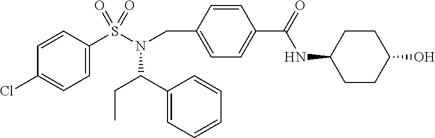

The title compound (170 mg, 62%) was obtained from (S)-4-((4-chloro-N-(1-phenylpropyl)phenylsulfonamido)methyl)benzoic acid (222 mg, 0.5 mmol) and trans-4-aminocyclohexanol (69.1 mg, 0.600 mmol) according to the General Procedure for Synthesis of Amide of Example 1.

MS (m/z): 541.16 (M⁺+1)

Elemental Analysis: C₂₉H₃₃ClN₂O₄S:

Calcd: C, 64.37; H, 6.15; N, 5.18. Found: C, 64.09, H, 6.10, N, 5.11

### Example 106

### AD1097

### (S)-4-((4-cChloro-N-(1-phenylpropyl)phenylsulfonamido)methyl)-N-(tetrahydro-2H-pyran-4-yl)benzamide

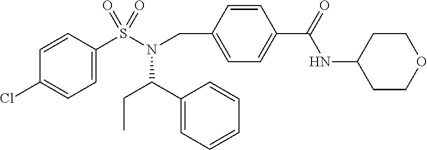

The title compound (200 mg, 76%) was obtained from (S)-4-((4-chloro-N-(1-phenylpropyl)phenylsulfonamido)methyl)benzoic acid (222 mg, 0.5 mmol) and tetrahydro-2H-pyran-4-amine (60.7 mg, 0.600 mmol) according to the General Procedure for Synthesis of Amide of Example 1.

MS (m/z): 527.16 (M⁺+1)

Elemental Analysis: C₂₈H₃₁ClN₂O₄S:

Calcd: C, 63.80; H, 5.93; N, 5.31. Found: C, 63.52, H, 5.80, N, 5.33

### Example 107

### AD1099

### Methyl 4-((4-chloro-N-((2R,3R)-1,3-dihydroxybutan-2-yl)phenylsulfonamido)-methyl)benzoate

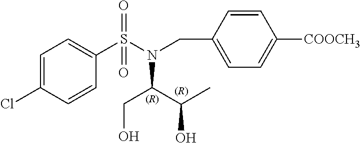

### Step 1

### 4-Chloro-N-((2R,3R)-1,3-dihydroxybutan-2-yl)benzenesulfonamide

A mixture of 4-chlorobenzene-1-sulfonyl chloride (3.82 g, 18.12 mmol), (2R,3R)-2-aminobutane-1,3-diol (L-threoninol, 2.0 g, 19.02 mmol) and potassium carbonate (6.26 g, 45.3 mmol) in anhydrous THF (20 mL) was stirred for 16 h. THF was removed in vacuo and the residue was partitioned between water (20 mL) and ethyl acetate (30 mL). The organic layer was separated and washed with 2N HCl, water, 10% sodium bicarbonate solution, water, brine and dried. Filtration and removal of solvent provided 3.99 g of product.

MS (m/z): 280.62 (M⁺+1)

### Step 2

### Methyl 4-((4-chloro-N-((2R,3R)-1,3-dihydroxybutan-2-yl)phenylsulfonamido)-methyl)benzoate

4-Chloro-N-((2S,3S)-1,3-dihydroxybutan-2-yl)benzenesulfonamide (280 mg, 1 mmol) was reacted with methyl-4-bromomethylbenzoate (241 mg, 1.05 mmol) according to the procedure described in Step 2 of Example 1. A white solid (192 mg, 44.8%) was isolated as product.

MS (m/z): 428.04 (M⁺+1)

Elemental Analysis: C₁₉H₂₂ClNO₆S:

Calcd: C, 53.33; H, 5.18; N, 3.27. Found: C, 53.30, H, 5.08, N, 3.21

### Example 108

### AD1101

### 4-Chloro-N-(4-(difluoromethoxy)-2-fluorobenzyl)-N-((2S,3S)-1,3-dihydroxybutan-2-yl)benzenesulfonamide

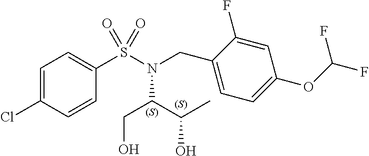

4-Chloro-N-((2S,3S)-1,3-dihydroxybutan-2-yl)benzenesulfonamide (261 mg, 0.934 mmol) and 1-(bromomethyl)-4-(difluoromethoxy)-2-fluorobenzene (250 mg, 0.980 mmol) were reacted according to the procedure described for Step 3, Example 1. After work up, 415 mg of crude product was purified by flash chromatography to yield 152 mg of pure product.

MS (m/z): 454.02 (M⁺+1)

Elemental Analysis: C₁₈H₁₉ClF₃NO₅S:

Calcd: C, 47.63; H, 4.22; N, 3.09. Found: C, 47.78, H, 4.00, N, 3.01

### Example 109

### AD1104

### Methyl 4-((5-chloro-N-((2S,3S)-1,3-dihydroxybutan-2-yl)thiophene-2-sulfonamido)methyl)benzoate

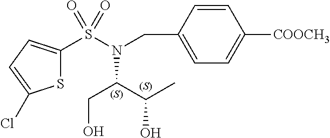

### Step 1

### 5-Chloro-N-((2S,3S)-1,3-dihydroxybutan-2-yl)thiophene-2-sulfonamide

To a solution of (2S,3S)-2-aminobutane-1,3-diol (D-threoninol, 880 mg, 8.12 mmol) in THF (15 mL) and potassium carbonate, 5-chlorothiophene-2-sulfonyl chloride (1679 mg, 7.73 mmol) in THF (3 mL) was added. The reaction mixture was stirred at room temperature for 6 h and then quenched with water (20 mL). THF was removed in vacuo, the residue was extracted with ethyl acetate and the organic layer was washed with water, brine and dried. After standard work-up, 1.378 g of crude liquid was purified by flash chromatography (hexane:ethyl acetate, 0-90%) to provide 1.20 g (54.3%) of product.

MS (m/z): 285.81 (M⁺+1)

### Step 2

### Methyl 4-((5-chloro-N-((2S,3S)-1,3-dihydroxybutan-2-yl)thiophene-2-sulfonamido)methyl)benzoate

5-Chloro-N-((2S,3S)-1,3-dihydroxybutan-2-yl)thiophene-2-sulfonamide

(286 mg, 1 mmol) was reacted with methyl-4-bromomethylbenzoate (241 mg, 1.05 mmol) according to the procedure described in Step 2 of Example 1. to give 95 mg (22.8%) of desired product.

MS (m/z): 434.02 (M⁺+1)

Elemental Analysis: C₁₇H₂₀ClNO₆S₂:

Calcd: C, 47.05; H, 4.65; N, 3.23. Found: C, 47.26, H, 4.42, N, 3.08

### Example 110

### AD1107

### (S)-4-((4-Chloro-N-(1-phenylpropyl)phenylsulfonamido)methyl)-N-(3-methoxypropyl)benzamide

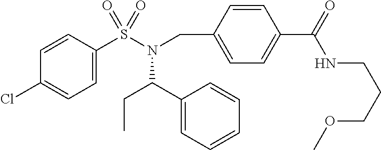

The title compound (180 mg, 70%) was prepared from of (S)-4-((4-chloro-N-(1-phenylpropyl)phenylsulfonamido)methyl)benzoic acid (222 mg, 0.500 mmol) and 3-methoxypropan-1-amine (53.5 mg, 0.600 mmol) according to the General Procedure for Synthesis of Amide of Example 1.

MS (m/z): 515.26 (M⁺+1)

Elemental Analysis: C₂₇H₃₁ClN₂O₄S:

Calcd: C, 62.96; H, 6.07; N, 5.44. Found: C, 62.71, H, 6.09, N, 5.44

### Example 111

### AD1109

### (S)-3-(4-((4-Chloro-N-(1-phenylpropyl)phenylsulfonamido)methyl)benzamido)-propanoic acid

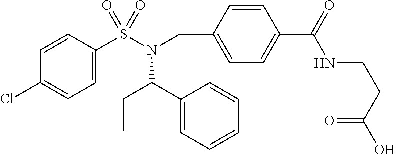

### Step 1

### (S)-Methyl 3-(4-((4-chloro-N-(1-phenylpropyl)phenylsulfonamido)methyl)-benzamido)propanoate

The title compound (251 mg, 63%) was prepared from of (S)-4-((4-chloro-N-(1-phenylpropyl)phenylsulfonamido)methyl)benzoic acid (333 mg, 0.75 mmol) and methyl 3-aminopropanoate hydrochloric salt (126 mg, 0.900 mmol) according to the General Procedure for Synthesis of Amide of Example 1.

MS (m/z): 529.14 (M⁺+1)

Elemental Analysis: C₂₇H₂₉ClN₂O₅S:

Calcd: C, 61.30; H, 5.53; N, 5.30. Found: C, 61.02, H, 5.33, N, 5.30

### Step 2

### (S)-3-(4-((4-Chloro-N-(1-phenylpropyl)phenylsulfonamido)methyl)benzamido)-propanoic acid

188 mg of (S)-methyl 3-(4-((4-chloro-N-(1-phenylpropyl)phenylsulfonamido)methyl)-benzamido)propanoate was hydrolyzed according to the procedure described in Step 3, Example 1. A white powder (158 mg, 86%) was generated as product.

MS (m/z): 515.14 (M⁺+1)

Elemental Analysis: C₂₆H₂₇ClN₂O₅S.H₂O:

Calcd: C, 58.58; H, 5.48; N, 5.26. Found: C, 58.76, H, 5.18, N, 5.17

### Example 112

### AD1115

### (S)-4-Chloro-N-((5-(hydroxymethyl)pyridin-2-yl)methyl)-N-(1-phenylpropyl)benzenesulfonamide

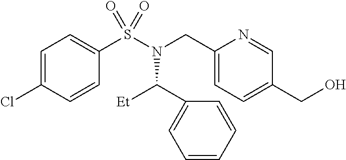 [SMILES: CC[C@@H](c1ccccc1)N(Cc1ccc(CO)cn1)S(=O)(=O)c1ccc(Cl)cc1]

(S)-Ethyl 6-((4-chloro-N-(1-phenylpropyl)phenylsulfonamido)methyl)nicotinate (0.095 g, 0.2 mmol) and NaBH4 (0.076 g, 2.000 mmol) were suspended in THF (4 mL) and then heated to 70° C. MeOH (0.5 mL) was then added and the mixture was stirred at 70° C. for 3 h. After cooling to room temperature, all solvent was evaporated and water (6 mL) was added. The mixture was extracted with EtOAc and purified by flash chromatography to give the desired product (66.8 mg, 77.5% yield).

¹H NMR (500 MHz, CDCl₃) δ7.60 (m, 2H), δ7.41 (m, 2H), δ7.23 (m, 4), δ7.01 (m, 2H), δ6.89 (d, J=1.0 Hz, 1H), δ 6.77 (d, J=1.0 Hz, 1H), δ4.90 (m, 1H), δ 4.70 (s, 2H), δ4.40 (d, J=13.5 Hz, 1H), δ4.01 (d, J=13.5 Hz, 1H), δ1.83 (m, 1H), δ1.75 (m, 1H), δ0.78 (t, J=6.5 Hz, 3H)

### Example 113

### AD1116

### (S)-4-Chloro-N-(1-phenylpropyl)-N-((6-(trifluoromethyl)pyridin-3-yl)methyl)benzenesulfonamide

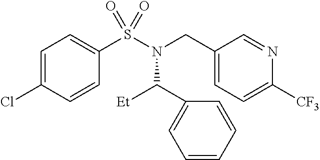

(6-(Trifluoromethyl)pyridin-3-yl)methanol (0.354 g, 2.000 mmol), (S)-4-chloro-N-(1-phenylpropyl)benzenesulfonamide (0.310 g, 1 mmol) and Ph₃P (0.577 g, 2.200 mmol) were dissolved in THF (5 mL). DIAD (0.456 ml, 2.200 mmol) was added dropwise and the mixture was then stirred at room temperature for 16 h. All solvent was evaporated, water (15 mL) was added, and then the mixture was extracted with EtOAc, purified by flash chromatography and a white solid (131 mg, 28% yield) was isolated as the desired product.

Mp 105-107° C.

¹H NMR (500 MHz, CDCl₃) δ8.28 (d, J=1.5 Hz, 1H), δ7.66 (m, 2H), 7.57 (m, 1H), 67.44 (m, 3H), 67.21 (m, 3H), 67.05 (m, 2H), 64.97 (dd, J1=7.5 Hz, J2=4.0 Hz, 1H), 64.37 (d, J=14 Hz, 1H), 64.28 (d, J=14 Hz, 1H), δ1.95 (m, 1H), δ1.70 (m, 1H), 60.80 (t, J=6.5 Hz, 3H)

### Example 114

### AD1117

### (S)-Methyl 4-((3,4-dichloro-N-(1-phenylpropyl)phenylsulfonamido)methyl)benzoate

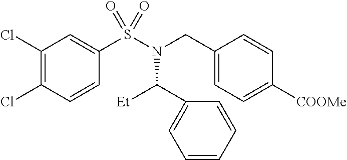 [SMILES: CC[C@@H](c1ccccc1)N(Cc1ccc(C(=O)OC)cc1)S(=O)(=O)c1ccc(Cl)c(Cl)c1]

(S)-3,4-Dichloro-N-(1-phenylpropyl)benzenesulfonamide (0.689 g, 2 mmol), methyl 4-(bromomethyl)benzoate (0.550 g, 2.400 mmol) and K₂CO₃ were stirred in DMF (5 mL) at room temperature for 16 h. The solvent was evaporated, water (15 mL) was added, and the mixture was then extracted with EtOAc and purified by flash chromatography to give a white solid (540 mg, 55% yield) as the final product.

Elemental Analysis: C₂₄H₂₃Cl₂NO₄S:

Calcd: C, 58.54, H, 4.71, N, 2.84. Found C, 58.63, H, 4.56, N, 2.72. Mp 98-99° C.

### Example 115

### AD1118

### (S)-Methyl 4-((3,4-difluoro-N-(1-phenylpropyl)phenylsulfonamido)methyl)benzoate

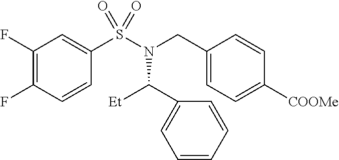 [SMILES: CC[C@@H](c1ccccc1)N(Cc1ccc(C(=O)OC)cc1)S(=O)(=O)c1ccc(F)c(F)c1]

(S)-3,4-Difluoro-N-(1-phenylpropyl)benzenesulfonamide (0.623 g, 2 mmol), methyl 4-(bromomethyl)benzoate (0.550 g, 2.400 mmol) and K₂CO₃ were stirred in DMF (5 mL) at room temperature for 16 h. All solvent was evaporated, water (15 mL) was added, and the mixture was extracted with EtOAc and purified by flash chromatography to give a white solid (480 mg, 52% yield) as the desired product.

Elemental Analysis: C₂₄H₂₃F₂NO₄S:

Calcd: C, 62.73, H, 5.05, N, 3.05. Found C, 62.86, H, 4.99, N, 2.94

Mp 103-105° C.

### Example 116

### AD1119

### (S)-4-((3,4-dichloro-N-(1-phenylpropyl)phenylsulfonamido)methyl)benzoic acid

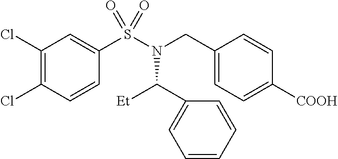 [SMILES: CC[C@@H](c1ccccc1)N(Cc1ccc(C(=O)O)cc1)S(=O)(=O)c1ccc(Cl)c(Cl)c1]

(S)-Methyl 4-((3,4-dichloro-N-(1-phenylpropyl)phenylsulfonamido)methyl)benzoate (0.246 g, 0.5 mmol) and lithium hydroxide hydrate (0.063 g, 1.500 mmol) were stirred at 50° C. for 16 h. THF was evaporated and water (20 mL) was added. The mixture was extracted with ether to remove impurities and then the mixture was adjusted to pH 2. Extraction with EtOAc followed by standard work-up procedures gave a white solid as product (207 mg, 86% yield).

Mp 111-112° C.

¹H NMR (500 MHz, CDCl₃) δ7.94 (d, J=6.5 Hz, 2H), δ7.49 (m, 2H), δ 7.26 (m, 4H), δ7.18 (d, J=7.0 Hz, 2H), δ7.07 (m, 2H), δ4.93 (dd, J1=7.5 Hz, J2=5.5 Hz, 1H), δ4.46 (d, J=13.5 Hz, 1H), δ4.19 (d, J=13.5 Hz, 1H), δ1.91 (m, 1H), δ1.79 (m, 1H), δ0.80 (t, J=6.5 Hz, 3H)

### Example 117

### AD1120

### (S)-4-((3,4-difluoro-N-(1-phenylpropyl)phenylsulfonamido)methyl)benzoic acid

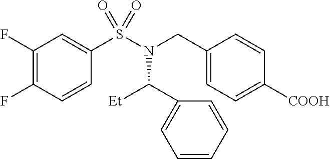 [SMILES: CC[C@@H](c1ccccc1)N(Cc1ccc(C(=O)O)cc1)S(=O)(=O)c1ccc(F)c(F)c1]

(S)-Methyl 4-((3,4-difluoro-N-(1-phenylpropyl)phenylsulfonamido)methyl)benzoate (0.230 g, 0.5 mmol) and lithium hydroxide hydrate (0.063 g, 1.500 mmol) were stirred at 50° C. for 16 h. THF was evaporated and water (20 mL) was added. The mixture was extracted with ether to remove impurities, the pH was adjusted to pH 2, extracted with EtOAc and dried to give the titled compound (150 mg, 67% yield).

Mp 108-109° C.

¹H NMR (500 MHz, CDCl₃) δ7.89 (d, J=6.5 Hz, 2H), δ7.45 (m, 2H), δ 7.25 (m, 4H), δ7.18 (d, J=7.0 Hz, 2H), δ7.02 (m, 2H), δ4.89 (dd, J1=7.5 Hz, J2=5.5 Hz, 1H), δ4.48 (d, J=13.5 Hz, 1H), δ4.13 (d, J=13.5 Hz, 1H), δ1.90 (m, 1H), δ1.76 (m, 1H), δ0.77 (t, J=6.5 Hz, 3H)

### Example 118

### AD1121

### 4-((4-Chloro-N-((2S,3S)-1,3-dihydroxybutan-2-yl)phenylsulfonamido)methyl)benzoic acid

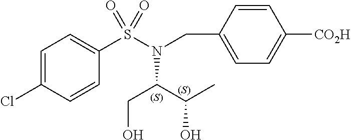

To a solution of methyl 4-((4-chloro-N-((2S,3S)-1,3-dihydroxybutan-2-yl)phenylsulfonamido)methyl)benzoate (106 mg, 0.248 mmol) in THF (4 mL) was added a solution of lithium hydroxide hydrate (52.0 mg, 1.239 mmol) in water (1 mL). After stirring the reaction at 50° C. for 6.5 h, THF was removed in vacuo, and 2N HCl was added to acidify the mixture to pH 2. Extraction with ethyl acetate followed by standard work-up procedure yielded 91 mg of the desired product.

MS (m/z): 414.06 (M⁺+1)

Elemental Analysis: C₁₈H₂₀ClNO₆S:

Calcd: C, 52.24; H, 4.87; N, 3.38. Found: C, 52.35, H, 4.86, N, 3.28

Mp 182-184° C.

### Example 119

### AD1123

### (S)-5-((4-Chloro-N-(1-phenylpropyl)phenylsulfonamido)methyl)-2-methoxybenzoic acid

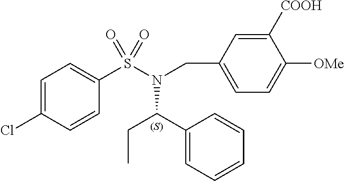

To a solution of ((S)-methyl 5-((4-chloro-N-(1-phenylpropyl)phenylsulfonamido)methyl)-2-methoxybenzoate (185 mg, 0.379 mmol) in THF (4 mL) was added an aqueous solution of lithium hydroxide monohydrate (63.6 mg, 1.516 mmol) in water (1 mL). The reaction mixture was stirred and heated at 60° C. for 8 h. After cooling the reaction mixture to room temperature, the THF was removed in vacuo, 2N HCl was added to adjust the mixture to pH 2 and the mixture was extracted with ethyl acetate. The organic layer was separated and washed with water (2 mL×2), brine and dried. Filtration and concentration yielded crude product which was triturated with diethyl ether to give 158 mg of the title compound.

MS (m/z): 474.40 (M⁺+1)

Elemental Analysis: C₂₄H₂₄ClNO₅S.⅓ H₂O:

Calcd: C, 60.06; H, 5.18; N, 2.92. Found: C, 60.13; H, 5.05; N, 2.90

### Example 120

### AD1123

### (S)-5-((4-Chloro-N-(1-phenylpropyl)phenylsulfonamido)methyl)-2-methoxybenzoic acid

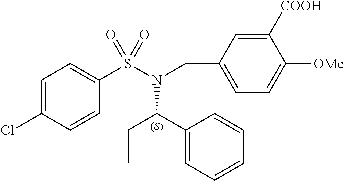

To a solution of ((S)-methyl 5-((4-chloro-N-(1-phenylpropyl)phenylsulfonamido)methyl)-2-methoxybenzoate (185 mg, 0.379 mmol) in THF (4 mL) was added an aqueous solution of lithium hydroxide monohydrate (63.6 mg, 1.516 mmol) in water (1 mL). The reaction mixture was stirred and heated at 60° C. for 8 h. After cooling the reaction mixture to room temperature, the THF was removed in vacuo, 2N HCl was added to adjust the mixture to pH 2 and the mixture was extracted with ethyl acetate. The organic layer was separated and washed with water (2 mL×2), brine and dried. Filtration and concentration yielded crude product which was triturated with diethyl ether to give 158 mg of the title compound.

MS (m/z): 474.40 (M⁺+1)

Elemental Analysis: C₂₄H₂₄ClNO₅S.⅓ H2O:

Calcd: C, 60.06; H, 5.18; N, 2.92. Found: C, 60.13; H, 5.05; N, 2.90

### Example 121

### AD1124

### (S)-4-((4-Chloro-N-(1-phenylpropyl)phenylsulfonamido)methyl)-2-fluorobenzoic acid

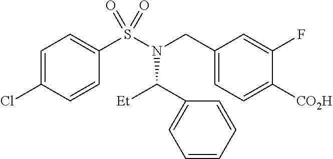 [SMILES: CC[C@@H](c1ccccc1)N(Cc1ccc(C(=O)O)c(F)c1)S(=O)(=O)c1ccc(Cl)cc1]

(S)-Methyl 4-((4-chloro-N-(1-phenylpropyl)phenylsulfonamido)methyl)-2-fluorobenzoate (240 mg, 0.504 mmol) and lithium hydroxide hydrate (63.5 mg, 1.513 mmol) were stirred at 50° C. for 16 h. The THF was evaporated and water (20 mL) was added. The mixture was extracted with ether to remove impurities and the mixture was adjusted the pH 2. Extraction with EtOAc following standard work-up procedures yielded the desired product (374 mg, 81% yield).

Mp 95-97° C.

¹H NMR (500 MHz, CDCl₃) δ7.75 (m, 1H), δ7.66 (m, 2H), δ7.45 (m, 2H), δ7.23 (m, 4H), δ7.01 (m, 1H), δ6.95 (m, 1H), δ6.87 (m, 1H), δ4.92 (m, 1H), δ4.41 (d, J=14.0 Hz, 1H), δ4.13 (d, J=14.0 Hz), δ1.90 (m, 1H), δ1.72 (m, 1H), δ0.78 (t, J=6.0 Hz, 3H)

### Example 122

### AD1125

### (S)-Methyl 4-((4-chloro-N-(1-(4-(trifluoromethyl)phenyl)propyl)phenylsulfonamido)methyl)benzoate

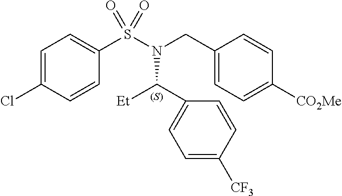

Methyl 4-((4-chlorophenylsulfonamido)methyl)benzoate (0.816 g, 2.400 mmol), (R)-1-(4-(trifluoromethyl)phenyl)propan-1-ol (0.408 g, 2 mmol) and Ph₃P (0.629 g, 2.400 mmol) were dissolved in THF and the mixture was cooled to −20° C. DIAD (0.495 ml, 2.400 mmol) was then added in one portion and the mixture was then stirred at room temperature for 16 h. Evaporation of the solvent and extraction with EtOAc following standard work-up procedures and purification by flash chromatography gave the desired product (632 mg, 60% yield).

Elemental Analysis: C₂₅H₂₃ClF₃NO₄S:

Calcd: C, 57.09, H, 4.41, N, 2.66. Found C, 56.96, H, 4.36, N, 2.66

Mp 123-125° C.

### Example 123

### AD1128

### (R)-Methyl 4-((4-chloro-N-(1-(4-(trifluoromethyl)phenyl)propyl)phenylsulfonamido)methyl)benzoate

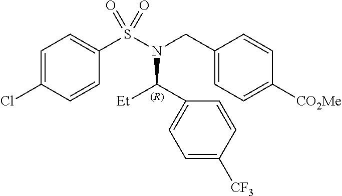

Methyl 4-((4-chlorophenylsulfonamido)methyl)benzoate (0.816 g, 2.400 mmol), (S)-1-(4-(trifluoromethyl)phenyl)propan-1-ol (0.408 g, 2 mmol) and Ph₃P (0.629 g, 2.400 mmol) were dissolved in THF and the mixture was cooled to −20° C. DIAD (0.495 ml, 2.400 mmol) was added in one portion and the mixture was then stirred at room temperature for 16 h. The solvent was evaporated and the mixture was extracted with EtOAc and followed standard work-up procedures. Purification by flash chromatography yielded desired product (654 mg, 62% yield).

Elemental Analysis: C₂₅H₂₃ClF₃NO₄S:

Calcd: C, 57.09, H, 4.41, N, 2.66. Found C, 56.91, H, 4.39, N, 2.55

Mp 120-121° C.

### Example 124

### AD1127

### (S)-Methyl 4-((4-chloro-N-(1-phenylpropyl)phenylsulfonamido)methyl)-2,3-difluorobenzoate

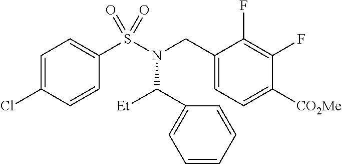

(S)-4-Chloro-N-(1-phenylpropyl)benzenesulfonamide (779 mg, 2.52 mmol), methyl 4-(bromomethyl)-2,3-difluorobenzoate (800 mg, 3.02 mmol) and K₂CO₃ were stirred in DMF at room temperature for 16 h. The solvent was evaporated, water (20 mL) was added and the mixture was extracted with EtOAc following standard work-up procedure to give the desired product (772 mg, 62% yield).

Elemental Analysis: C₂₄H₂₂ClF₂NO₄S:

Calcd: C, 58.36; H, 4.49, N, 2.84. Found C, 58.28, H, 4.44, N, 2.75

Mp 87-89° C.

### Example 125

### AD1128

### (S)-4-((4-Chloro-N-(1-(4-(trifluoromethyl)phenyl)propyl)phenylsulfonamido)methyl)benzoic acid

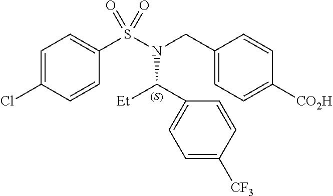

(S)-Methyl 4-((4-chloro-N-(1-(4-(trifluoromethyl)phenyl)propyl)phenylsulfonamido)-methyl)benzoate (400 mg, 0.761 mmol) and lithium hydroxide hydrate (191 mg, 4.56 mmol) were stirred at room temperature for 24 h. The solvent was evaporated, water (10 mL) was added, and the mixture was then adjusted to pH 2. Extraction with EtOAc and following standard work-up procedure, the desired product (360 mg, 93% yield) was isolated.

Elemental Analysis: C₂₄H₂₁ ClF₃NO₄S:

Calcd: C, 56.31, H, 4.13, N, 2.74. Found C, 56.22, H, 4.09, N, 2.56

Mp 101-103° C.

### Example 126

### AD1129

### (R)-4-((4-Chloro-N-(1-(4-(trifluoromethyl)phenyl)propyl)phenylsulfonamido)methyl)benzoic acid

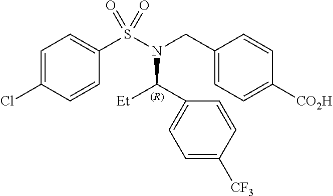

(R)-Methyl 4-((4-chloro-N-(1-(4-(trifluoromethyl)phenyl)propyl)phenylsulfonamido)-methyl)benzoate (400 mg, 0.761 mmol) and lithium hydroxide hydrate (191 mg, 4.56 mmol) were stirred at room temperature for 24 h. The solvent was evaporated, water (10 mL) was added and the mixture was adjusted to pH 2. Extraction with EtOAc following the standard work-up procedure gave the desired product (370 mg, 94% yield).

Elemental Analysis: (C₂₄H₂₁ClF₃NO₄S):

Calcd: C, 56.31, H, 4.13, N, 2.74. Found C, 56.25, H, 4.12, N, 2.62

Mp 105-107° C.

### Example 127

### AD1130

### (S)-4-((4-Chloro-N-(1-phenylpropyl)phenylsulfonamido)methyl)-2,3-difluorobenzoic acid

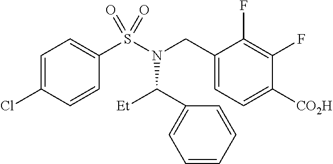 [SMILES: CC[C@@H](c1ccccc1)N(Cc1ccc(C(=O)O)c(F)c1F)S(=O)(=O)c1ccc(Cl)cc1]

(S)-Methyl 4-((4-chloro-N-(1-phenylpropyl)phenylsulfonamido)methyl)-2,3-difluorobenzoate (480 mg, 0.972 mmol) and lithium hydroxide hydrate (163 mg, 3.89 mmol) were stirred at 50° C. for 16 h. Th solvent was evaporated, water (8 mL) was added, the mixture was extracted with ether and the aqueous phase was adjust to pH2 using 4N HCl. The mixture was extracted with EtOAc, dried and the solvent was evaporated in vacuo to give the desired product (324 mg, 69% yield)

Elemental Analysis: C₂₃H₂₀ClF₂NO₄S:

Calcd: C, 57.56, H, 4.20, N, 2.92. Found C, 57.73, H, 4.24, N, 2.60

Mp 127-129° C.

### Example 128

### AD1134

### 4-((4-Chloro-N—((S)-1-phenylpropyl)phenylsulfonamido)methyl)-N-((R)-1-methoxypropan-2-yl)benzamide

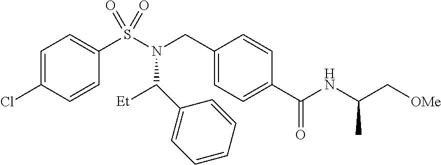

To a solution of 4-((4-chloro-N—((S)-1-phenylpropyl)phenylsulfonamido)methyl)-N—((R)-1-hydroxypropan-2-yl)benzamide (125 mg, 0.249 mmol) in THF at −40° C. was added a suspension of sodium hydride (11 mg, prewashed with hexane) in THF. The mixture was then stirred at −10° C. for about 40 min. Iodomethane (39 mg, 0.274 mmol) was then added and the reaction mixture was warmed to room temperature and stirred for 16 h. Purification by flash chromatography yielded the desired product.

MS (m/z): 515.3

### Example 129

### AD1135

### (S)-4-((4-Chloro-N-(1-phenylpropyl)phenylsulfonamido)methyl)-N-(2-methoxyethyl)benzamide

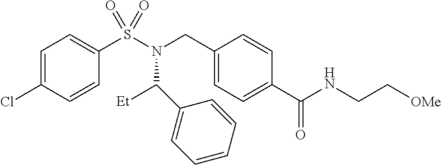

To the solution of (S)-4-((4-chloro-N-(1-phenylpropyl)phenylsulfonamido)methyl)-N-(2-methoxyethyl)benzamide (65 mg, 0.130 mmol) and iodomethane (57.6 μl, 0.924 mmol) in acetonitrile (1.5 mL) was added silver monooxide (214 mg, 0.924 mmol). The mixture was stirred in the dark for 16 h. Purification by flash chromatography gave the desired product in 42% yield.

MS (m/z): 501.0

### Example 130

### AD1137

### (S)-4-((4-Chloro-N-(1-phenylpropyl)phenylsulfonamido)methyl)-N-ethylbenzamide

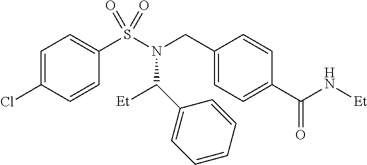

To a −20° C. solution of (S)-4-((4-chloro-N-(1-phenylpropyl)phenylsulfonamido)methyl)benzoic acid (120 mg, 0.270 mmol) and triethylamine (82 mg, 0.811 mmol), methanesulfonyl chloride (31.0 mg, 0.270 mmol) was added and the mixture was stirred for 1 hr at −20° C. Ethanamine (135 μA, 0.270 mmol) in THF was then added to the reaction mixture and the resulting solution was slowly warmed to room temperature. Usual workup of organic extraction and concentration in vacuo, followed by flash chromatography purification gave the desired product.

MS (m/z): 471.3

### Example 131

### AD1156

### 4-Chloro-N-(4-cyano-2-fluorobenzyl)-N-((2S,3S)-1,3-dihydroxybutan-2-yl)-benzenesulfonamide

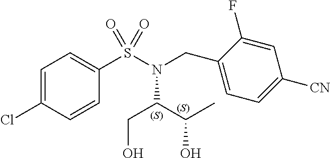

A mixture of 4-chloro-N-((2S,3S)-1,3-dihydroxybutan-2-yl)benzenesulfonamide (280 mg, 1.0 mmol), 4-(bromomethyl)-3-fluorobenzonitrile (225 mg, 1.05 mmol) and cesium carbonate (652 mg, 2 mmol) in DMF (2 mL) was stirred at room temperature for 2.5 h. Water (12 mL) was added and the product was extracted with ethyl acetate. The organic layer was separated and washed with water, brine and dried. Filtration and concentration yielded 414 mg of crude product which was purified by flash chromatography (hexane:ethyl acetate, 0-60%) to yield 205 mg (49.6%) of desired product.

MS (m/z): 413.05 (M⁺+1)

Elemental Analysis: C₁₈H₁₈ClFN₂O₄S:

Calcd: C, 52.36; H, 4.39; N, 6.79. Found: C, 52.39; H, 4.32; N, 6.67

Mp 112-114° C.

### Example 132

### AD1157

### 4-Chloro-N-(4-cyanobenzyl)-N-((2S,3S)-1,3-dihydroxybutan-2-yl)-benzene-sulfonamide

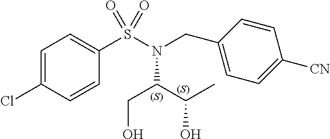

A mixture of 4-chloro-N-(2S,3S)-1,3-dihydroxybutan-2-yl)benzenesulfonamide (280 mg, 1.0 mmol), 4-(bromomethyl)benzonitrile (206 mg, 1.05 mmol) and cesium carbonate (652 mg, 2 mmol) in DMF (2 mL) was stirred at room temperature for 2.5 h. Water (12 mL) was then added and the product was extracted with ethyl acetate. The organic layer was separated and washed with water, brine and dried. Filtration and concentration yielded 372 mg of crude product which was purified by flash chromatography (hexane:ethyl acetate, 0-70%) to yield 210 mg (53%) of desired product.

MS (m/z) 395.05 (M⁺+1)

Elemental Analysis: C₁₈H₁₉ClN₂O₄S:

Calcd: C, 54.75; H, 4.85; N, 7.09. Found: C, 54.92; H, 5.06; N, 7.39

Mp 124-126° C.

### Example 133

### AD1158

### (R)-Methyl 5-((4-chloro-N-(2-hydroxy-1-phenylethyl)phenylsulfonamido)methyl)-2-methoxybenzoate

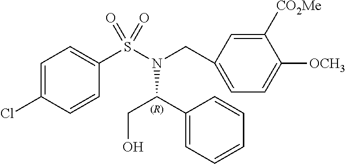

To a mixture of (R)-4-chloro-N-(2-hydroxy-1-phenylethyl)benzenesulfonamide (320 mg, 1.026 mmol) and methyl 5-(bromomethyl)-2-methoxybenzoate (293 mg, 1.129 mmol) in DMF (3 mL) was added cesium carbonate (669 mg, 2.053 mmol). The reaction mixture was stirred at room temperature for 2 h and then water (12 mL) was added. The mixture was extracted with ethyl acetate and the organic layer was separated and washed with water, brine and dried. Filtration and concentration yielded 512 mg of crude product that was purified by flash chromatography (hexane:ethyl acetate, 0-40%) to yield 262 mg of product.

MS (m/z): 490.08 (M⁺+1)

Elemental Analysis: C₂₄H₂₄ClNO₆S:

Calcd: 58.83; H, 4.94; N, 2.86. Found: C, 58.88; H, 4.85; N, 2.79

Mp 60-62° C.

### Example 134

### AD1160

### (R)-5-((4-Chloro-N-(2-hydroxy-1-phenylethyl)phenylsulfonamido)methyl)-2-methoxybenzoic acid

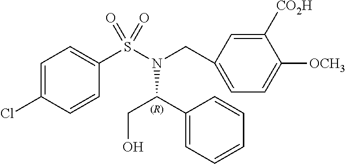

To a solution of (R)-methyl 5-((4-chloro-N-(2-hydroxy-1-phenylethyl)phenyl-sulfonamido)methyl)-2-methoxybenzoate (160 mg, 0.327 mmol) in THF (4 mL) was added lithium hydroxide hydrate (54.8 mg, 1.306 mmol) in water (1 mL). The reaction mixture was stirred and heated in a sealed pressure tube for 5 h. The reaction was then cooled to room temperature and THF was removed in vacuo. Water (1 mL) was added and 4 N HCl was added dropwise to acidify the mixture to pH 2. The mixture was then extracted with ethyl acetate and the organic layer was separated and washed with water, brine and dried. Filtration and concentration provided 134 mg (86%) of a white solid.

MS (m/z): 476.07 (M⁺+1)

Elemental Analysis: C₂₃H₂₂ClNO₆S:

Calcd: C, 58.04; H, 4.66; N, 2.94. Found: C, 57.78; H, 4.55; N, 2.86

Mp 79-80° C.

### Example 135

### AD972

### tert-Butyl 4-((5-chloro-N-(1,3-dihydroxypropan-2-yl)thiophene-2-sulfonamido)-methyl)benzoate

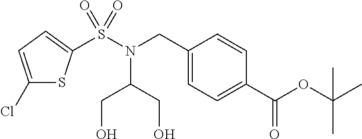 [SMILES: CC(C)(C)OC(=O)c1ccc(CN(C(CO)CO)S(=O)(=O)c2ccc(Cl)s2)cc1]

### Step 1

### tert-Butyl 4-((5-chloro-N-(2,2-dimethyl-1,3-dioxan-5-yl)thiophene-2-sulfonamido)-methyl)benzoate

A mixture of 5-chloro-N-(2,2-dimethyl-1,3-dioxan-5-yl)thiophene-2-sulfonamide (305 mg, 0.978 mmol, see Example 42), tert-butyl 4-(bromomethyl)benzoate (292 mg, 1.076 mmol) and Cs₂CO₃ in DMF (3 mL) was stirred at room temperature for 16 h. The reaction was quenched with water (12 mL) and extracted with ethyl acetate. The organic layer was separated and washed with water and brine, and dried over sodium sulfate. Filtration and concentration provided 564 mg of crude product which was purified by flash chromatography (hexane:ethyl acetate, 0-30%) to give 218 mg of final product.

MS (m/z): 502.3 (M⁺+1)

### Step 2

### tert-Butyl 4-((5-chloro-N-(1,3-dihydroxypropan-2-yl)thiophene-2-sulfonamido)-methyl)benzoate

A mixture of tert-butyl 4-((5-chloro-N-(2,2-dimethyl-1,3-dioxan-5-yl)thiophene-2-sulfonamido)methyl)benzoate (213 mg, 0.424 mmol), toluenesulfonic acid mono hydrate (89 mg, 0.46 mmol) and MeOH (0.5 mL) in THF was stirred at room temperature for 3.5 h. The reaction was quenched with aqueous sodium carbonate solution to pH 11-12. The organic solvents were removed in vacuo and the residue was partitioned between ethyl acetate and water. The organic layer was separated and washed with water, brine and dried. Filtration and concentration provided 201 mg of crude product which was purified by flash chromatography (hexane:ethyl acetate, 0-60%) to yield 145 mg (74.0%) of white solid as the desired product.

MS (m/z): 462.30 (M⁺+1)

Elemental Analysis: C₁₉H₂₄ClNO₆S₂:

Calcd: C, 49.40; H, 5.24; N, 3.03. Found: C, 49.68; H, 5.36; N, 2.99

### Example 136

### AD993

### (S)-Methyl 4-((4-ethoxy-N-(1-phenylpropyl)phenylsulfonamido)methyl)benzoate

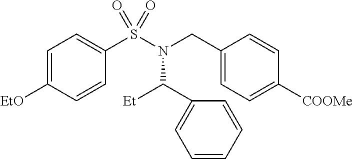

To a stirred solution of (S)-4-ethoxy-N-(1-phenylpropyl)benzenesulfonamide (0.489 g, 1.5 mmol) and methyl 4-(bromomethyl)benzoate (0.412 g, 1.800 mmol) in dry DMF (6 mL) was added K₂CO₃ at room temperature. The mixture was then stirred for 16 h, the solvent was evaporated and water (20 mL) was added. The mixture was extracted with EtOAc and the organic layers were concentrated in vacuo to afford a residue that was then purified by flash chromatography to give the title compound (423.0 mg, 60.3% yield).

Mp 102-104° C.

¹H NMR (500 MHz, CDCl₃) δ7.84 (d, J=7.0 Hz, 2H), δ7.66 (d, J=7.0 Hz, 2H), δ7.20 (m, 3H), δ7.15 (d, J=6.5 Hz, 2H), δ6.97 (m, 2H), δ6.89 (m, 2H), δ4.89 (m, 1H), δ4.46 (d, J=13.5 Hz, 1H), δ4.08 (m, 2H), δ4.02 (d, J=13.5 Hz, 1H), δ3.85 (s, 3H), δ1.80 (m, 1H), δ1.71 (m, 1H), δ1.45 (t, J=6.0 Hz, 3H), δ0.75 (t, J=6.0 Hz, 3H)

### Example 137

### AD992

### (S)—N-(4-Cyanobenzyl)-4-ethoxy-N-(1-phenylpropyl)benzenesulfonamide

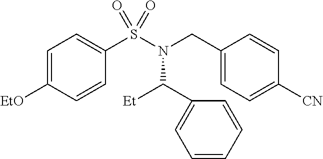

To a stirred solution of (S)-4-ethoxy-N-(1-phenylpropyl)benzenesulfonamide (0.326 g, 1 mmol) and 4-(bromomethyl)benzonitrile (0.235 g, 1.200 mmol) in dry DMF (4 mL) was added K₂CO₃ at room temperature. The mixture was then stirred for 16 h, the solvent was evaporated and water (20 mL) was added. The mixture was extracted with EtOAc and the organic layers were concentrated in vacuo to afford a residue that was then purified by flash chromatography to give the title compound (292.0 mg, 67.2% yield).

Mp 97-98° C.

¹H NMR (500 MHz, CDCl₃) δ7.67 (m, 2H), δ7.44 (m, 2H), δ7.16 (m, 5H), δ6.98 (m, 2H), δ6.91 (m, 2H), δ4.91 (m, 1H), δ4.38 (d, J=14.0 Hz, 1H), δ4.12 (d, J=14.0 Hz, 1H), δ4.09 (m, 2H), δ1.85 (m, 1H), δ1.68 (m, 1H), δ1.46 (t, J=6.0 Hz, 3H), δ0.76 (t, J=6.5 Hz, 3H).

### Example 138

### AD994

### (S)-Methyl 4-(N-(4-cyanobenzyl)-N-(1-phenylpropyl)sulfamoyl)benzoate

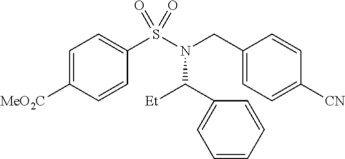

To a stirred solution of (S)-methyl 4-(N-(1-phenylpropyl)sulfamoyl)benzoate (200 mg, 0.600 mmol) and 4-(bromomethyl)benzonitrile (141 mg, 0.720 mmol) in dry DMF (4 mL) was added K₂CO₃ at room temperature. The mixture was then stirred for 16 h, the solvent was evaporated and water (10 mL) was added. The mixture was extracted with EtOAc and the organic layers were concentrated in vacuo to afford a residue that was then purified by flash chromatography to give the title compound (185 mg, 69% yield).

¹H NMR (500 MHz, CDCl₃) δ7.88 (d, J=7.0 Hz, 2H), δ7.59 (d, J=7.0 Hz, 2H), δ7.17-7.58 (m, 7H), δ7.16 (d, J=10.0 Hz, 2H), δ3.90 (s, 3H), δ3.80 (t, J=12.5 Hz, 2H), δ3.50 (m, 1H), δ3.25 (d, J=12.0 Hz, 2H), δ2.0 (m, 1H), δ1.84 (m, 1H), δ0.89 (t, J=4.5 Hz, 3H).

### Example 139

### AD999

### Methyl 4-((4-chloro-N-(1-p-tolylpropyl)phenylsulfonamido)methyl)benzoate

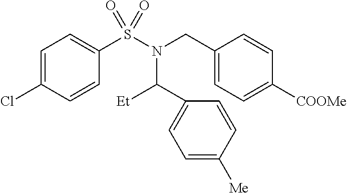 [SMILES: CCC(c1ccc(C)cc1)N(Cc1ccc(C(=O)OC)cc1)S(=O)(=O)c1ccc(Cl)cc1]

The title compound (114 mg, 24% yield) was prepared from methyl 4-((4-chlorophenylsulfonamido) methyl)benzoate and 1-p-tolylpropan-1-ol following the same procedure as that for the synthesis of Example 176.

¹H NMR (500 MHz, CDCl₃) δ7.88 (d, J=7.0 Hz, 2H), δ7.64 (d, J=7.0 Hz, 2H), δ7.40 (m, 2H), δ7.19 (m, 2H), δ7.02 (d, J=7.0 Hz, 2H), δ6.84 (d, J=7.0 Hz, 2H), δ4.85 (m, 1H), δ4.50 (d, J=6.0 Hz, 1H), δ4.05 (d, J=6.0 Hz, 1H), δ3.90 (s, 3H), δ2.30 (s, 3H), δ1.79 (m, 1H), δ1.72 (m, 1H), δ0.75 (t, J=6.5 Hz, 3H)

### Example 140

### AD1070

### (S)-Methyl 4-((4-chloro-N-(2-hydroxy-1-phenylethyl) phenylsulfonamido)methyl)benzoate

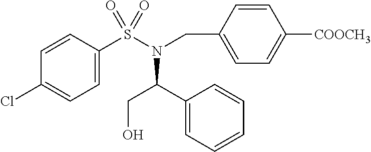 [SMILES: COC(=O)c1ccc(CN([C@H](CO)c2ccccc2)S(=O)(=O)c2ccc(Cl)cc2)cc1]

Example 140 was prepared via the procedure described in Step 2 of Example 95.

### Example 141

### AD1170

### (S)-Methyl 4-((4-chloro-N-(1-(4-chlorophenyl)ethyl)phenylsulfonamido)methyl)benzoate

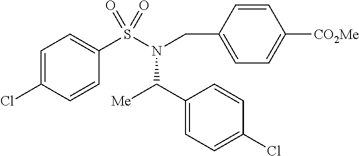

Example 141 was prepared via the General Method described in Scheme 1.

MS (m/z): 477.1

Mp 67-69° C.

### Example 141

### AD1171

### (S)-4-((4-Chloro-N-(1-(4-chlorophenyl)ethyl)phenylsulfonamido)methyl)benzoic acid

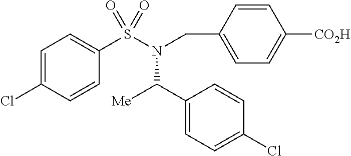

Example 141 was prepared via the General Method described in Scheme 1.

MS (m/z): 463.0

Mp 163-165° C.

### Example 142

Additional examples of compounds of Formula I, which may be made using the methods described herein, optionally modified by methods within the skill of one in the art, include the following:

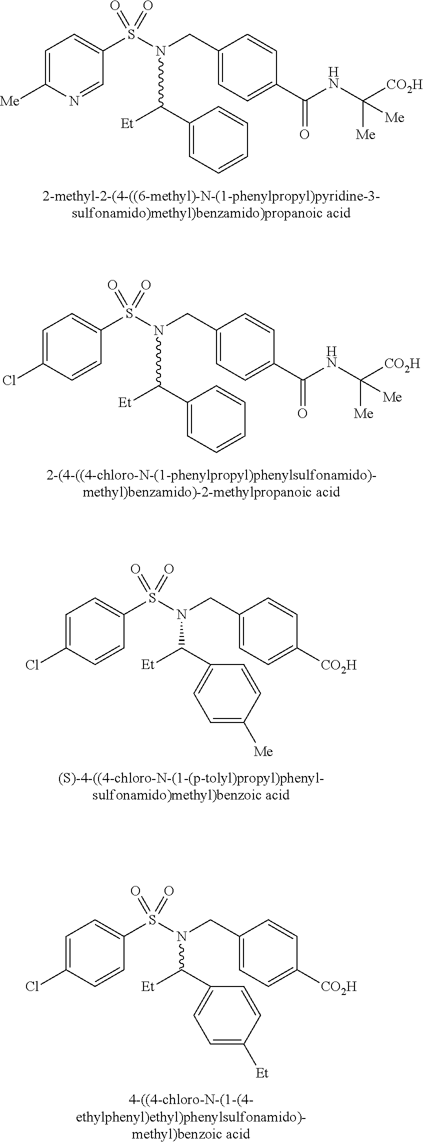

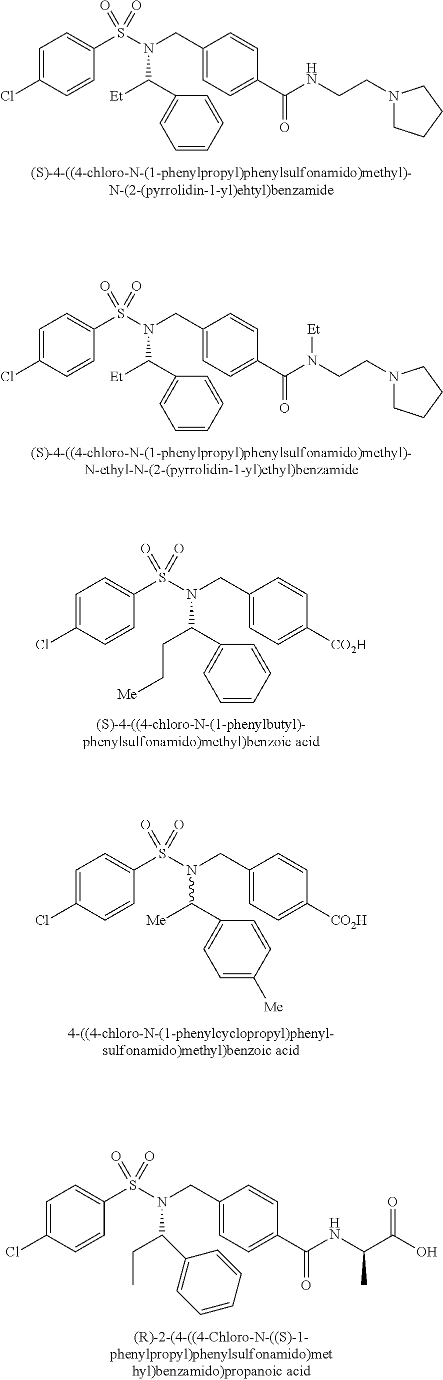

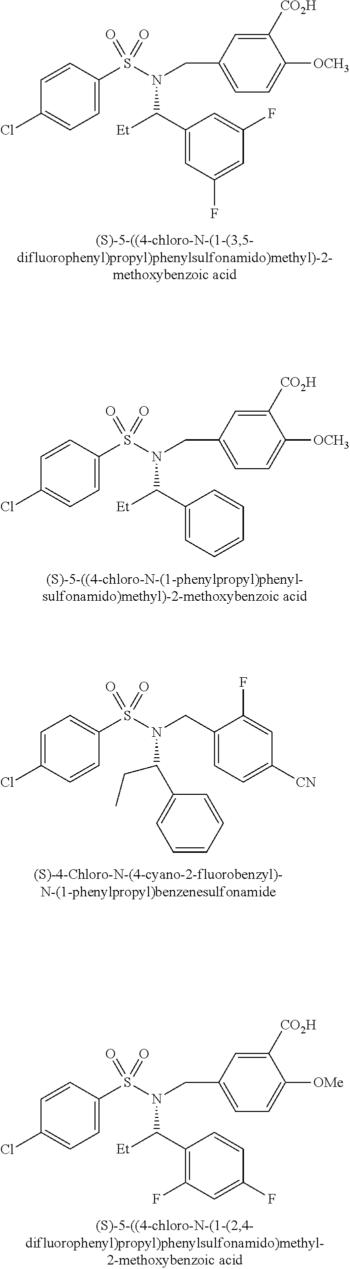

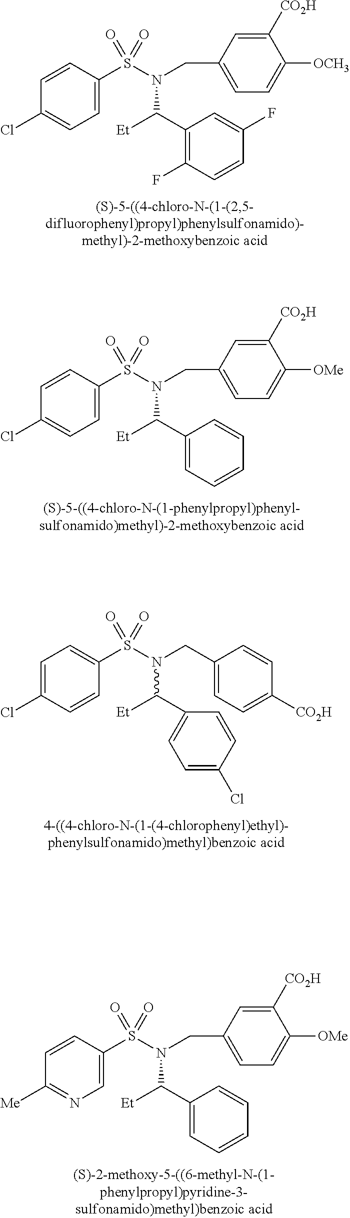

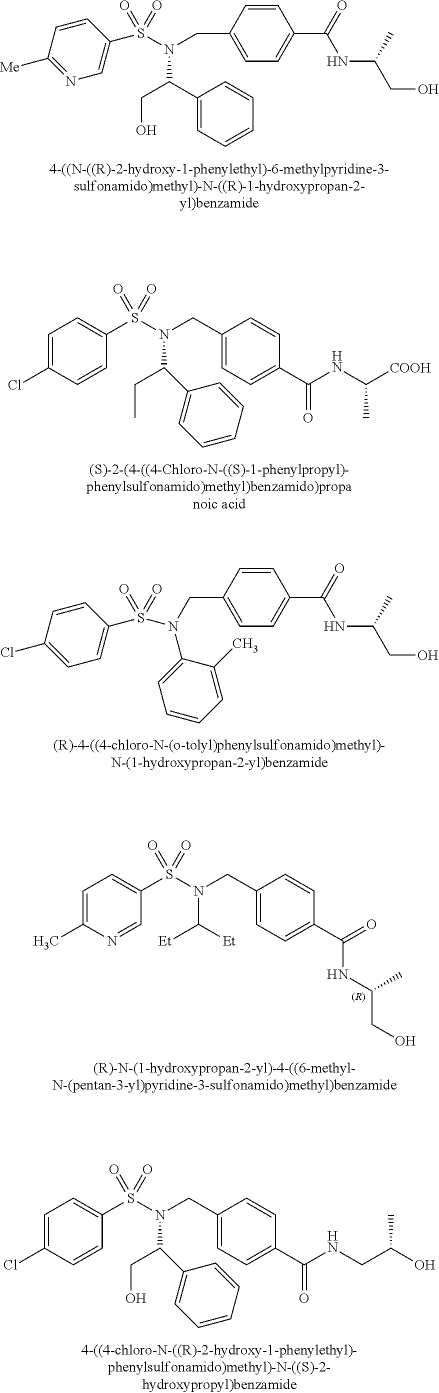

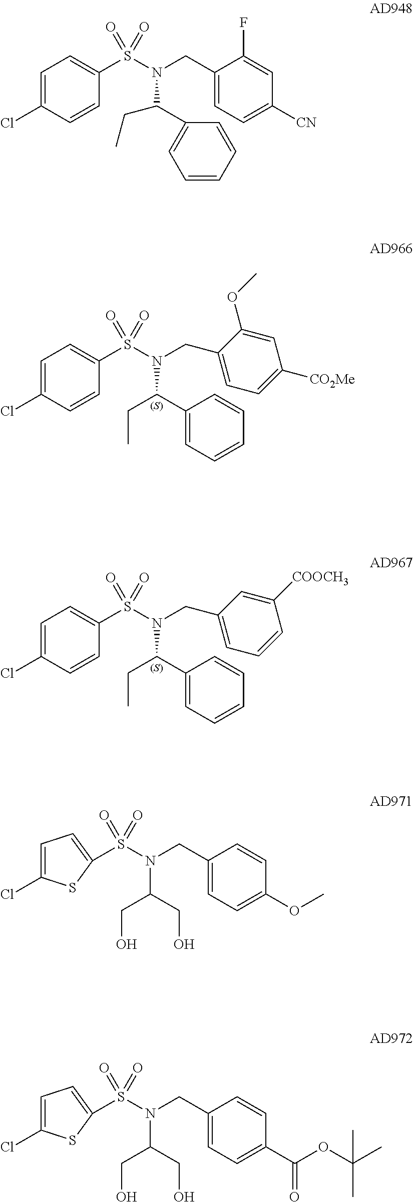

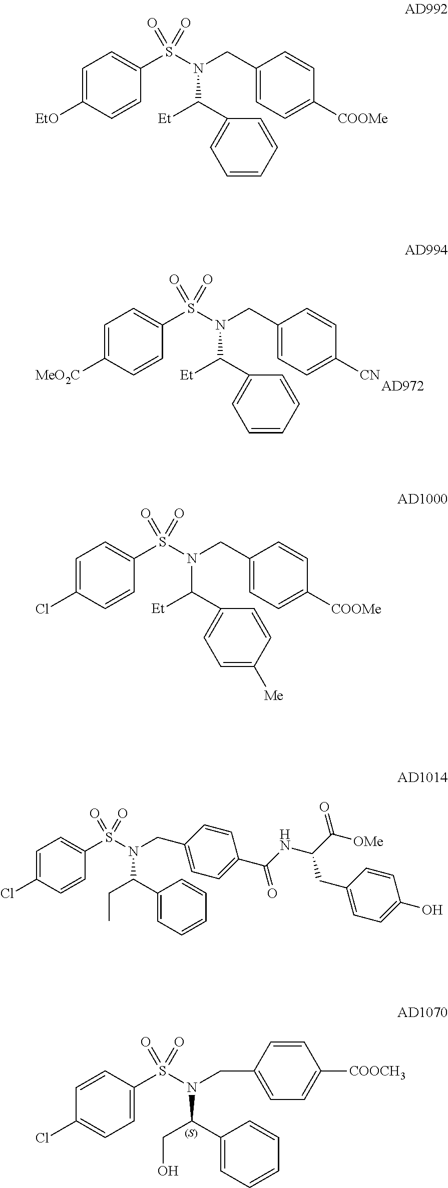

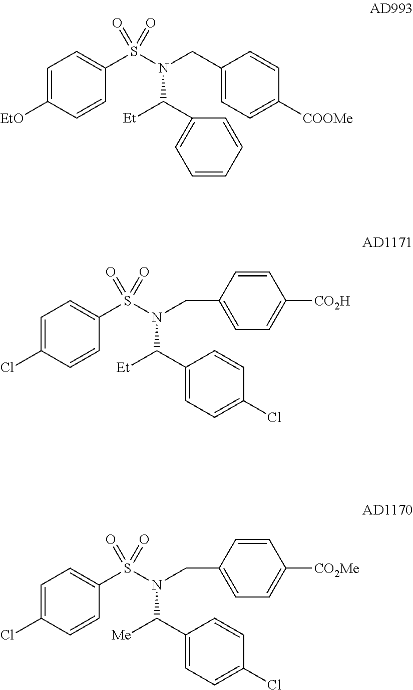

A number of embodiments of the invention have been described. Nevertheless, it will be understood that various modifications may be made without departing from the spirit and scope of the invention. Accordingly, other embodiments are within the scope of the following claims.

1. A compound of the formula (I): 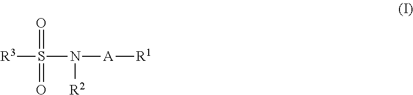
wherein:
R¹ is: 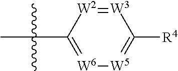
wherein:
W², W³, W⁵, and W⁶ are defined according to (A) or (B) below:
(A)
each of W² and W⁶ is independently selected from CH and C(halo); and
each of W³ and W⁵ is independently selected from CH, C(halo), and CR′; wherein R′ is —C(O)OH, —C(O)O(C₁-C₆ alkyl), or —CN; or
(B)
one or two of W², W³, W⁵, and W⁶ are N; and the others are independently selected from CH and C(halo);
R⁴ is selected from any of the substituents delineated in (i)-(v) immediately below:
(i) halo; —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; —NO₂; —SO₃H; —P(O)(OH)₂; —OH, —SO₂(R⁴⁵); —NHC(O)R⁴¹, —NHSO₂R⁴¹, —SO₂N(R⁴²)(R⁴³); —C(O)NHCH(CH₂OH)₂, —C(O)NH(CH₂)₃COOH; OCH(CH₂OH)₂;
(ii) C₁-C₆ alkoxy, C₁-C₆ thioalkoxy, C₁-C₆ haloalkoxy, C₁-C₆ halothioalkoxy, C₁-C₆ alkyl, C₁-C₆ haloalkyl, each of which is optionally substituted with from 1-3 (e.g., 1-2 or 1) substituents independently selected from —OH, C₁-C₃ alkoxy, —C(O)OH, —C(O)O(C₁-C₆ alkyl), and —CN;
(iii) heterocyclyl or heterocyclyloxy, each containing from 3-8 ring atoms, wherein from 1-2 of the ring atoms is independently selected from N, NH, N(C₁-C₆ alkyl), O, and S; and wherein said heterocyclyl or heterocyclyloxy is optionally substituted with from 1-3 independently selected R^{a};
(iv) heterocycloalkenyl or heteroaryl, each containing 5 ring atoms, wherein from 1-4 of the ring atoms is independently selected from N, NH, N(C₁-C₆ alkyl), O, and S; and wherein said heteroaryl ring is optionally substituted with from 1-3 independently selected R^{b}; and
(v) hydrogen;
R⁴¹ is C₁-C₈ alkyl, C₁-C₈ haloalkyl, or benzyl optionally substituted with from 1-3 R^{b};
each of R⁴² and R⁴³ is, independently:
(i) hydrogen; or
(ii) C₁-C₈ alkyl; C₁-C₈ haloalkyl; C₃-C₈ cycloalkyl; and heterocyclyl containing from 3-8 ring atoms, wherein from 1-2 of the ring atoms is independently selected from N, NH, N(C₁-C₆ alkyl), O, and S; and wherein each of said alkyl, haloalkyl, cycloalkyl, and heterocyclyl is optionally substituted with from 1-3 R^{e};
or
R⁴²—N—R⁴² together forms a saturated ring having 5 or 6 ring atoms, in which from 1 or 2 ring atoms, in addition to the N that occurs between R⁴² and R⁴³, is/are optionally a heteroatom independently selected from NH, N(alkyl), O, or S; and wherein said saturated ring is optionally substituted with from 1-3 R^{e};
R⁴⁴ is hydrogen, C₁-C₈ alkyl, or C₁-C₈ haloalkyl;
R⁴⁵ is C₁-C₈ alkyl or C₁-C₈ haloalkyl;
provided that only one of R⁴ and R′ or only one of R⁴ and two occurrences of R′ can be —C(O)OH, —C(O)O(C₁-C₆ alkyl), or —CN;
A is C(R^{A})₂, wherein each occurrence of R^{A} is independently selected from hydrogen and —CH₃;
R² is: 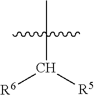
R⁵ is:
(i) C₆-C₁₀ aryl, which is optionally substituted with from 1-3 independently selected R^{c};
or
(ii) heteroaryl containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{c}; or
R⁶ is C₁-C₆ alkyl or C₁-C₆ haloalkyl, each of which is optionally substituted with a substituent selected from —OH and —CN; or
R³ is:
(i) C₆-C₁₀ aryl, which is optionally substituted with from 1-3 independently selected R^{d}; or
(ii) heteroaryl, each containing from 5-10 ring atoms, wherein from 1-6 of the ring atoms is independently selected from N, NH, N(C₁-C₆ alkyl), O, and S; and wherein said heteroaryl ring is optionally substituted with from 1-3 independently selected R^{d};
R^{a} at each occurrence is, independently, selected from halo, —OH, C₁-C₆ alkoxy, C₁-C₆ thioalkoxy, C₁-C₆ haloalkoxy, C₁-C₆ thiohaloalkoxy, C₁-C₆ alkyl, C₁-C₆ haloalkyl, and —CN;
R^{b} at each occurrence is, independently selected from halo, —OH, C₁-C₆ alkoxy, C₁-C₆ thioalkoxy, C₁-C₆ haloalkoxy, C₁-C₆ thiohaloalkoxy, C₁-C₆ alkyl, C₁-C₆ haloalkyl, —NH₂, —NH(C₁-C₆ alkyl), N(C₁-C₆ alkyl)₂, —NHC(O)(C₁-C₆ alkyl), —CN; and —NO₂;
R^{c} at each occurrence is independently selected from the substituents delineated in (aa), (bb) and (cc) below:
(aa) halo; C₁-C₆ alkoxy; C₁-C₆ haloalkoxy; C₁-C₆ thioalkoxy; C₁-C₆ thiohaloalkoxy; C₁-C₆ alkyl, C₁-C₆ haloalkyl, —NH(C₁-C₆ alkyl), N(C₁-C₆ alkyl)₂, —NHC(O)(C₁-C₆ alkyl), wherein the alkyl portion of each is optionally substituted with —OH, C₁-C₃ alkoxy, —C(O)OH, —C(O)O(C₁-C₆ alkyl), and —CN;
(bb) —OH; —CN; nitro; —NH₂; azido; C₂-C₄ alkenyl; C₂-C₄ alkynyl; —C(O)H; —C(O)(C₁-C₆ alkyl); C(O)OH; —C(O)O(C₁-C₆ alkyl); —C(O)NH₂—SO₂(C₁-C₆ alkyl); —SO₂(C₁-C₆ haloalkyl); —C(O)NR′″R″″, —SO₂NR′→R″″, —SO₂NH₂, —NHCO(C₁-C₆ alkyl), —NHSO₂(C₁-C₆ alkyl), whereby R′″ and R″″ is independently selected from H, C₁-C₆ alkyl, C₁-C₆ haloalkyl;
(cc) C₃-C₆ cycloalkyl or heterocyclyl containing from 5-6 ring atoms, wherein from 1-2 of the ring atoms of the heterocyclyl is independently selected from N, NH, N(C₁-C₆ alkyl), NC(O)(C₁-C₆ alkyl), O, and S; and wherein each of said cycloalkyl and heterocyclyl is optionally substituted with from 1-3 independently selected C₁-C₄ alkyl groups;
and
R^{d} at each occurrence is, independently selected from halo, C₁-C₆ alkoxy, C₁-C₆ thioalkoxy, C₁-C₆ haloalkoxy, C₁-C₆ thiohaloalkoxy, C₁-C₆ alkyl, C₁-C₆ haloalkyl, —CN; COOH, NO₂, C(O)(C₁-C₆ alkyl), C(O)(C₁-C₆ haloalkyl), azido, NCS, —CH₂OH, amino, NR′″R″″, N-azidinyl, N-morpholinyl, S(C₁-C₆ alkyl), —SO₂(C₁-C₆ alkyl), —C(O)NR′″R″″, —SO₂NR′″R″″, —SO₂NH₂, —NHCO(C₁-C₆ alkyl), and —NHSO₂(C₁-C₆ alkyl), whereby R′″ and R″″ is independently selected from H, C₁-C₆ alkyl, C₁-C₆ haloalkyl;
provided that when R² is unsubstituted alkyl or alkyl that is substituted with one or more —OH, then R⁴ cannot be hydrogen, halo, or C₁-C₆ alkoxy, except that when R² is unsubstituted alkyl or alkyl that is substituted with one or more —OH, then R⁴ can be C₁-C₆ alkoxy when either R′ is —C(O)OH, —C(O)O(C₁-C₆ alkyl); or when two or more of W², W³, W⁵, and W⁶ are each independently C(halo);
or a pharmaceutically acceptable salt thereof.

2. The compound of claim 1, wherein W², W³, W⁵, and W⁶ are defined according to definition (A).

3. (canceled)

4. The compound according to claim 1, wherein each of W², W³, W⁵, and W⁶ is CH.

5-12. (canceled)

13. The compound according to claim 1, wherein R⁴ is selected from —CO₂H; —C(O)OR⁴¹; —NHC(O)OR⁴¹; —N(CH₃)C(O)OR⁴¹; —C(O)N(R⁴²)(R⁴³); —C(O)R⁴⁴; —CN; and —SO₂(R⁴⁵).

14. The compound according to claim 13, wherein R⁴ is —CO₂H.

15. The compound according to claim 13, wherein R⁴ is —CO₂R⁴¹.

16. (canceled)

17. The compound according to claim 13, wherein R⁴ is —SO₂(R⁴⁵).

18. (canceled)

19. The compound according to claim 13, wherein R⁴ is —C(O)N(R⁴²)(R⁴³).

20-27. (canceled)

28. The compound of claim 1, wherein R⁵ is C₆-C₁₀ aryl, which is optionally substituted with from 1-3 independently selected R^{c}.

29. The compound of claim 28, wherein R⁵ is phenyl, which is optionally substituted with from 1-3 independently selected R^{c}.

30. The compound of claim 29, wherein, R⁵ is unsubstituted phenyl.

31. The compound according to claim 1, wherein R⁶ is C₁-C₆ alkyl, which is optionally substituted with a substituent selected from —OH and —CN.

32. The compound of claim 31, wherein R⁶ is —CH₂CH₃ or —CH₃.

33-38. (canceled)

39. The compound according to claim 1, wherein the carbon attached to R⁵ and R⁶ has the S configuration.

40. The compound according to claim 1, wherein R³ is C₆-C₁₀ aryl, which is optionally substituted with from 1-3 independently selected R^{d}.

41. (canceled)

42. The compound of claim 40, wherein R³ is 4-chloro-phenyl, 4-fluoro-phenyl, or 2,4-difluorophenyl.

43-46. (canceled)

47. The compound according to claim 1, wherein A is CH₂.

48. A pharmaceutical composition comprising a compound of formula (I), or a pharmaceutically acceptable salt thereof, as claimed in claim 1, and a pharmaceutically acceptable carrier.

49. A method for treating a neurodegenerative disorder subject having, or at risk of having a neurodegenerative disorder, which comprises administering to the subject having, or at risk of having a neurodegenerative disorder a therapeutically effective amount of a compound of formula (I), or a pharmaceutically acceptable salt thereof, as claimed in claim 1.

50-52. (canceled)

53. The method of claim 49, wherein the neurodegenerative disorder is Alzheimer's disease.
